(19) United States
(12) Patent Application Publication
(10) Pub. No.: US 2025/0354118 A1
(43) Pub. Date: Nov. 20, 2025

(54) COMPOSITIONS AND METHODS FOR NK CELL DIFFERENTIATION

(71) Applicant: Umoja Biopharma, Inc., Seattle, WA (US)

(72) Inventors: Teisha J. Rowland, Seattle, WA (US); Samantha O'HARA, Seattle, WA (US); David T. VEREIDE, Seattle, WA (US); Ashley M. YINGST, Seattle, WA (US)

(73) Assignee: Umoja Biopharma, Inc., Seattle, WA (US)

(21) Appl. No.: 18/872,801

(22) PCT Filed: Jun. 9, 2023
(86) PCT No.: PCT/US2023/068217

§ 371 Date: Dec. 6, 2024

Related U.S. Application Data
(60) Provisional application No. 63/350,755, filed on Jun. 9, 2022.

(51) Int. Cl. C12N 5/0783; C12N 5/0789

(52) U.S. Cl. CPC C12N 5/0646; C12N 5/0647; C12N 2501/115; C12N 2501/125; C12N 2501/145; C12N 2501/155; C12N 2501/165; C12N 2501/2302; C12N 2501/2307; C12N 2501/2312; C12N 2501/2315; C12N 2501/2318; C12N 2501/26; C12N 2501/727; C12N 2501/998; C12N 2501/999; C12N 2506/02; C12N 2506/45

(57) ABSTRACT

Provided arm xenogenic-free methods and compositions for generating hematopoietic progenitors and natural killer (NK) cells.

### CROSS-REFERENCE TO RELATED APPLICATIONS

This application claims the benefit of priority to U.S. Provisional Patent Application No. 63/350,755, filed Jun. 9, 2022, the disclosure of which is incorporated herein by reference in its entirety for all purposes.

### BACKGROUND

Natural Killer (NK) cells are a type of cytotoxic innate lymphoid cells generally identified as positive for the cell surface protein CD56 (CD56+) and other markers and as having cytotoxic activity.

NK cells for use in immunotherapy can be obtained from primary sources such as peripheral blood or umbilical cord blood. Artificial sources for NK cells include pluripotent stem cells, including induced pluripotent stem cells (iPSCs), which are cells derived from somatic cells (generally fibroblasts or peripheral blood mononuclear cells [PBMCs]), and human embryonic stem cells (hESCs), either induced to become capable of unlimited proliferation and of differentiation into other cell types when subjected to appropriate differentiation conditions. From iPSCs, NK cells may be derived by sequentially differentiating the iPSCs into hematopoietic progenitor cells (HPCs), also termed hematopoietic stem cells (HSCs).

Once obtained, primary NK or iPSC-NK cells can be expanded ex vivo before administration to patients. Methods for differentiating iPSCs into NK cells often involves the use of feeder cells or media with serum. To provide cells suitable for in vivo administration, there remains a need for compositions and methods related to generating NK cells without using xenogenic factors, such as animal-derived raw materials like fetal bovine serum (FBS) and/or feeder cells.

### SUMMARY

The present disclosure is based, at least in part, on the discovery of a method for differentiating stem cells into hematopoietic progenitors and NK cells that is xenogenic free. Without wishing to be bound by theory, the xenogenic free method described herein results in cells suitable for in vivo administration.

In some embodiments, the disclosure provides a method for generating a population of CD34+/CD43+/CD45+ cells, comprising contacting a population of stem cells with a differentiation media comprising a bone morphogenetic protein (BMP) pathway activator, a fibroblast growth factor (FGF), and a vascular endothelial growth factor (VEGF), for a period of time sufficient to generate the population of CD34+/CD43+/CD45+ cells from the population of stem cells.

In some embodiments, the disclosure provides a method for differentiation a population of stem cells into a population of hematopoietic progenitors, comprising contacting the population of stem cells with a differentiation media comprising a bone morphogenetic protein (BMP) pathway activator, a fibroblast growth factor (FGF), and a vascular endothelial growth factor (VEGF), for a period of time sufficient to differentiation the population of stem cells into the population of hematopoietic progenitors.

In some embodiments, the population of hematopoietic progenitors comprises CD34+/CD43+/CD45+ cells.

In some embodiments, the BMP pathway activator is BMP4. In some embodiments, the FGF is FGF2. In some embodiments, the VEGF is VEGF-165. In some embodiments, the differentiation media comprises Rho-associated coiled coil forming protein serine/threonine kinase (ROCK) inhibitor. In some embodiments, the ROCK inhibitor is Y27632.

In some embodiments, the differentiation media comprises stem cell factor (SCF). In some embodiments, the differentiation media comprises thrombopoietin (TPO). In some embodiments, the differentiation media comprises a low-density lipoprotein (LDL). In some embodiments, the differentiation media comprises a phosphoinositide 3-kinase (PI3K) inhibitor.

In some embodiments, the PI3K inhibitor is LY294002. In some embodiments, the differentiation media comprises a pyrimido-[4,5-b]-indole derivative. In some embodiments, the pyrimido-[4,5-b]-indole derivative is UM729. In some embodiments, the differentiation media comprises an aryl hydrocarbon receptor (AhR) antagonist. In some embodiments, the AhR antagonist is StemRegenin 1 (SR1).

In some embodiments, the differentiation media comprises the BMP pathway activator, the FGF, the VEGF, and the ROCK inhibitor.

In some embodiments, the differentiation media comprises the BMP pathway activator, the FGF, the VEGF, SCF, TPO, the LDL and the inhibitor of PI3K.

In some embodiments, the differentiation media comprises the BMP pathway activator, the FGF, the VEGF, SCF, TPO, the LDL, the PI3K inhibitor, the pyrimido-[4,5-b]-indole derivative, and the AhR antagonist.

In some embodiments, the method comprises contacting the population of stem cells with the differentiation media for 1-5 days, wherein the differentiation media comprises the BMP pathway activator, the FGF, the VEGF, and optionally the ROCK inhibitor.

In some embodiments, the method comprises (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, and the PI3K inhibitor.

In some embodiments, the method comprises (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, the PI3K inhibitor, the pyrimido-[4,5-b]-indole derivative, and the AhR antagonist.

In some embodiments, the differentiation media comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

In some embodiments, the stem cells are induced pluripotent stem cells (iPSCs).

In some embodiments, the stem cells are human embryonic stem cells (hESCs).

In some embodiments, the disclosure provides a method for generating a population of CD43+/CD45+/CD56+/LFA1+ cells, comprising contacting a population of CD34+/CD43+/CD45+ cells with a media comprising SCF, interleukin-7 (IL-7), IL-12, IL-15, FMS-like tyrosine kinase 3 ligand (FLT3L), a pyrimido-[4,5-b]-indole derivative, and an AhR inhibitor, for a period of time sufficient to generate the population of CD43+/CD45+/CD56+/LFA1+ cells from the population of CD34+/CD43+/CD45+.

In some embodiments, the disclosure provides a method of differentiating a population of hematopoietic progenitors into a population of Natural Killer (NK) cells, comprising contacting the population of hematopoietic progenitors with a differentiation media comprising SCF, IL-7, IL-12, IL-15, FLT3L, a pyrimido-[4,5-b]-indole derivative, and an AhR inhibitor, for a period of time sufficient to differentiate the population of hematopoietic progenitors into the population of NK cells.

In some embodiments, the disclosure provides a method of differentiating a population of common lymphoid progenitors (CLPs) into a population of Natural Killer (NK) cells, comprising contacting the population of hematopoietic progenitors with a differentiation media comprising SCF, IL-7, IL-12, IL-15, FLT3L, a pyrimido-[4,5-b]-indole derivative, and an AhR inhibitor, for a period of time sufficient to differentiate the population of hematopoietic progenitors into the population of NK cells.

In some embodiments, the pyrimido-[4,5-b]-indole derivative is UM729. In some embodiments, the AhR inhibitor is SR1.

In some embodiments, the media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

In some embodiments, the period of time is 11-25 days.

In some embodiments, the method comprises maturing the population of NK cells with a maturation media comprising (i) IL-12, IL-15 and IL-18, or (ii) IL-12, IL-2 and IL-18.

In some embodiments, the differentiation media and/or maturation media is serum free.

In some embodiments, the method is xenogenic-free.

In some embodiments, the disclosure provides a method of generating a population of NK cells, comprising:
(a) obtaining a population of stem cells;
(b) contacting the population of stem cells with a first media comprising a BMP pathway activator, an FGF, a VEGF, and optionally an inhibitor of ROCK, for a period of time sufficient to generate embryoid bodies;
(c) contacting the embryoid bodies with a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor, for a period of time sufficient to generate a population of hematopoietic progenitors;
(d) contacting the population of hematopoietic progenitors with a second differentiation media comprising SCF, IL-7, IL-12, IL-15, FLT3L, a pyrimido-[4,5-b]-indole derivative, and an AhR inhibitor, for a period of time sufficient to generate the population of NK cells.

In some embodiments, the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

In some embodiments, each media of steps (b)-(d) is serum free.

In some embodiments, the method is xenogenic-free.

In some embodiments, the first media, the first differentiation media, and the second differentiation media each comprise the same base media.

In some embodiments, the first media, the first differentiation media, and the second differentiation media each comprise different base media.

In some embodiments, the first differentiation media and the second differentiation media each comprise the same base media, and the first media comprises a base media different from the first and second differentiation media.

In some embodiments, the first differentiation media and the second differentiation media each comprise a base media comprising Iscove's modified dulbecco's medium, bovine serum albumin, recombinant human insulin, human transferrin, and 2-mercaptoethanol.

In some embodiments, the period of time of step (b) is 1-5 days, the period of time of step (c) is 3-15 days, and the period of time of step (d) is 11-25 days.

In some embodiments, steps (a)-(d) occur within 35-45 days.

In some embodiments, the method comprises (e) expanding the population of NK cells with a maturation media comprising (i) IL-12, IL-15 and IL-18, or (ii) IL-12, IL-2 and IL-18.

In some embodiments, the stem cells are induced pluripotent stem cells (iPSCs) or human embryonic stem cells (hESCs).

In some embodiments, the population of hematopoietic progenitors comprises about 30% to about 50% CD34+/CD43+/CD45+ cells.

In some embodiments, the population of NK cells comprises about 60% to about 100% CD43+/CD45+/CD56+/LFA1+ cells.

In some embodiments, the method comprises expanding the population of NK cells, wherein the population of NK cells expands about 10- to about 350-fold.

In some embodiments, the population of stem cells is genetically engineered or edited.

In some embodiments, the population of NK cells is genetically engineered or edited.

In some embodiments, the disclosure provides a population of cells comprising hematopoietic progenitors produced by the methods of the disclosure.

In some embodiments, the hematopoietic progenitors are CD34+/CD43+/CD45+.

In some embodiments, the population of cells comprise 30-50% hematopoietic progenitors.

In some embodiments, the disclosure provides a population of cells comprising NK cells produced by the methods of the disclosure.

In some embodiments, the NK cells are CD43+/CD45+/CD56+/LFA1+.

In some embodiments, the population of cells comprises 60-100% NK cells.

In some embodiments, the disclosure provides a hematopoietic progenitor differentiation media comprising a serum-free base media, a BMP pathway activator, an FGF, a VEGF, SCF, TPO, LDL, and a PI3K inhibitor.

In some embodiments, the BMP pathway activator is BMP4, the FGF is FGF2, the VEGF is VEGF-165, and the PI3K inhibitor is LY294002.

In some embodiments, the media comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

In some embodiments, the disclosure provides an NK cell differentiation media comprising a serum-free base media, SCF, IL-7, IL-12, IL-15, FLT3L, a pyrimido-[4,5-b]-indole derivative, and an AhR inhibitor.

In some embodiments, the pyrimido-[4,5-b]-indole derivative is UM729 and the AhR inhibitor is SR1.

In some embodiments, the media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

In some embodiments, the disclosure provides a kit comprising the hematopoietic progenitor differentiation media and instructions for contacting a population of stem cells with the hematopoietic progenitor differentiation media for a period of time sufficient to generate a population of cells comprising hematopoietic progenitors.

In some embodiments, the period of time is 1-15 days.

In some embodiments, the disclosure provides a kit comprising the NK cell differentiation media and instructions for contacting a population of hematopoietic progenitors with the NK cell differentiation media for a period of time sufficient to generate a population of cells comprising NK cells.

In some embodiments, the period of time is 11-25 days.

In some embodiments, the disclosure provides a kit comprising the hematopoietic progenitor differentiation media and the NK cell differentiation media, and instructions for contacting a population of stem cells with the hematopoietic progenitor differentiation media for a first period of time sufficient to generate a population of cells comprising hematopoietic progenitors, and contacting the population of cells comprising hematopoietic progenitors with the NK cell differentiation media for a second period of time sufficient to generate a population of cells comprising NK cells.

In some embodiments, the first period of time is 1-15 days, and the second period of time is 11-25 days.

In some embodiments, the kit further comprises a maturation media comprising a base media and (i) IL-12, IL-15 and IL-18, or (ii) IL-12, IL-2 and IL-18, and instructions for contacting the population of cells comprising NK cells for a period of time sufficient to mature the NK cells.

In some embodiments, the disclosure provides a composition to increase the yield ratio of hematopoietic progenitors from a population of stem cells, the composition comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor. In some embodiments, the disclosure provides a composition to increase the yield ratio of hematopoietic progenitors from a stem cell, the composition comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor.

In some embodiments, the hematopoietic progenitors comprise CD34+/CD43+/CD45+ cells.

In some embodiments, the BMP pathway activator is BMP4. In some embodiments, the FGF is FGF2. In some embodiments, the VEGF is VEGF-165. In some embodiments, the ROCK inhibitor is Y27632. In some embodiments, the composition further comprises stem cell factor (SCF). In some embodiments, the composition further comprises thrombopoietin (TPO). In some embodiments, the composition further comprises a low-density lipoprotein (LDL). In some embodiments, the composition further comprises a phosphoinositide 3-kinase (PI3K) inhibitor. In some embodiments, the PI3K inhibitor is LY294002. In some embodiments, the composition further comprises a pyrimido-[4,5-b]-indole derivative. In some embodiments, the pyrimido-[4,5-b]-indole derivative is UM729. In some embodiments, the composition further comprises an aryl hydrocarbon receptor (AhR) antagonist. In some embodiments, the AhR antagonist is StemRegenin 1 (SR1).

In some embodiments, the composition comprises the BMP pathway activator, the FGF, the VEGF, and the ROCK inhibitor.

In some embodiments, the composition comprises the BMP pathway activator, the FGF, the VEGF, SCF, TPO, the LDL and the inhibitor of PI3K.

In some embodiments, the composition comprises the BMP pathway activator, the FGF, the VEGF, SCF, TPO, the LDL, the PI3K inhibitor, the pyrimido-[4,5-b]-indole derivative, and the AhR antagonist.

In some embodiments, the composition comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

In some embodiments, the population of stem cells are induced pluripotent stem cells (iPSCs) In some embodiments, the population of stem cells are human embryonic stem cells (hESCs).

In some embodiments, the yield ratio of hematopoietic progenitor cells (HP) to stem cell (StC) (HP/StC) is about 2:1 to about 10:1. In some embodiments, the yield ratio of hematopoietic progenitor cells (HP) to stem cell (StC) (HP/StC) is about 5:1.

In some embodiments, the disclosure provides a method to increase the yield ratio of hematopoietic progenitor cells from a population of stem cells, comprising contacting the population of stem cells with a differentiation media comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor for a period of time sufficient to differentiate the population of stem cells into the hematopoietic progenitors. In some embodiments, the disclosure provides a method to increase the yield ratio of hematopoietic progenitor cells from a stem cell, comprising contacting the stem cell with a differentiation media comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor for a period of time sufficient to differentiate the population of stem cells into the hematopoietic progenitors.

In some embodiments, the hematopoietic progenitors comprise CD34+/CD43+/CD45+ cells.

In some embodiments, the BMP pathway activator is BMP4. In some embodiments, the FGF is FGF2. In some embodiments, the VEGF is VEGF-165. In some embodiments, the ROCK inhibitor is Y27632. In some embodiments, the differentiation media comprises stem cell factor (SCF). In some embodiments, the differentiation media comprises thrombopoietin (TPO). In some embodiments, the differentiation media comprises a low-density lipoprotein (LDL). In some embodiments, the differentiation media comprises a phosphoinositide 3-kinase (PI3K) inhibitor. In some embodiments, the PI3K inhibitor is LY294002. In some embodiments, the differentiation media comprises a pyrimido-[4,5-b]-indole derivative. In some embodiments, the pyrimido-[4,5-b]-indole derivative is UM729. In some embodiments, the differentiation media comprises an aryl hydrocarbon receptor (AhR) antagonist. In some embodiments, the AhR antagonist is StemRegenin 1 (SR1).

In some embodiments, the differentiation media comprises the BMP pathway activator, the FGF, the VEGF, and the ROCK inhibitor.

In some embodiments, the differentiation media comprises the BMP pathway activator, the FGF, the VEGF, SCF, TPO, the LDL and the inhibitor of PI3K.

In some embodiments, the differentiation media comprises the BMP pathway activator, the FGF, the VEGF, SCF, TPO, the LDL, the PI3K inhibitor, the pyrimido-[4,5-b]-indole derivative, and the AhR antagonist.

In some embodiments, the method comprises contacting the population of stem cells with the differentiation media for 1-5 days, wherein the differentiation media comprises the BMP pathway activator, the FGF, the VEGF, and optionally the ROCK inhibitor.

In some embodiments, the method comprises (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, and the PI3K inhibitor.

In some embodiments, the method comprises (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, the PI3K inhibitor, the pyrimido-[4,5-b]-indole derivative, and the AhR antagonist.

In some embodiments, the differentiation media comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

In some embodiments, the population of stem cells is a population of induced pluripotent stem cells (iPSCs). In some embodiments, the population of stem cells is a population of human embryonic stem cells (hESCs).

In some embodiments, the yield ratio of hematopoietic progenitor cells from the population of stem cells is about 2:1 to about 10:1. In some embodiments, the yield ratio of the population of hematopoietic progenitor cells from a stem cell is about 5:1.

In some embodiments, the disclosure provides a kit to increase the yield ratio of NK cells from a population of stem cells, wherein the kit comprises instructions for differentiating the population of stem cells into NK cells and:
(a) a first media comprising a BMP pathway activator, an FGF, a VEGF, and optionally an inhibitor of ROCK;
(b) a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor;
(c) a second differentiation media comprising SCF, IL-7, IL-12, IL-15, FLT3L, a pyrimido-[4,5-b]-indole derivative, and an AhR inhibitor.

In some embodiments, the disclosure provides a kit to increase the yield ratio of NK cells from a stem cell, wherein the kit comprises instructions for differentiating the stem cell into NK cells and:
(a) a first media comprising a BMP pathway activator, an FGF, a VEGF, and optionally an inhibitor of ROCK;
(b) a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor;
(c) a second differentiation media comprising SCF, IL-7, IL-12, IL-15, FLT3L, a pyrimido-[4,5-b]-indole derivative, and an AhR inhibitor.

In some embodiments, the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

In some embodiments, each media of (a)-(c) is serum free. In some embodiments, the media is xenogenic-free.

In some embodiments, the first media, the first differentiation media, and the second differentiation media each comprise the same base media.

In some embodiments, the first media, the first differentiation media, and the second differentiation media each comprise different base media.

In some embodiments, the first differentiation media and the second differentiation media each comprise the same base media, and the first media comprises a base media different from the first and second differentiation media.

In some embodiments, the first differentiation media and the second differentiation media each comprise a base media comprising Iscove's modified dulbecco's medium, bovine serum albumin, recombinant human insulin, human transferrin, and 2-mercaptoethanol.

In some embodiments, the kit further comprises a maturation media comprising (i) IL-12, IL-15 and IL-18, or (ii) IL-12, IL-2 and IL-18.

In some embodiments, the population of stem cells is a population of induced pluripotent stem cells (iPSCs) or a population of human embryonic stem cells (hESCs).

In some embodiments, the NK cells comprise about 60% to about 100% CD43+/CD45+/CD56+/LFA1+ cells.

In some embodiments, the population of stem cells is genetically engineered or edited

In some embodiments, the NK cells are genetically engineered or edited.

In some embodiments, the yield ratio of NK cells from a population of stem cells is about 2:1 to about 100:1. In some embodiments, the yield ratio of NK cells from a population of stem cells is about 35:1.

In some embodiments, the disclosure provides a method to increase the yield ratio of NK cells from a population of stem cells, comprising:
(a) obtaining a population of stem cells;
(b) contacting the population of stem cells with a first media comprising a BMP pathway activator, an FGF, a VEGF, and optionally an inhibitor of ROCK, for a period of time sufficient to generate embryoid bodies;
(c) contacting the embryoid bodies with a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor, for a period of time sufficient to generate a population of hematopoietic progenitors;
(d) contacting the population of hematopoietic progenitors with a second differentiation media comprising SCF, IL-7, IL-12, IL-15, FLT3L, a pyrimido-[4,5-b]-indole derivative, and an AhR inhibitor, for a period of time sufficient to generate the NK cells. 137. The method of claim 136, wherein the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

In some embodiments, the disclosure provides a method to increase the yield ratio of NK cells from a stem cell, comprising:
(a) obtaining a stem cell;
(b) contacting the stem cell with a first media comprising a BMP pathway activator, an FGF, a VEGF, and optionally an inhibitor of ROCK, for a period of time sufficient to generate embryoid bodies;
(c) contacting the embryoid bodies with a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor, for a period of time sufficient to generate a population of hematopoietic progenitors;
(d) contacting the population of hematopoietic progenitors with a second differentiation media comprising SCF, IL-7, IL-12, IL-15, FLT3L, a pyrimido-[4,5-b]-indole derivative, and an AhR inhibitor, for a period of time sufficient to generate the NK cells.

137. The method of claim 136, wherein the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

In some embodiments, each media of steps (b)-(d) is serum free. In some embodiments, the method is xenogenic-free.

In some embodiments, the first media, the first differentiation media, and the second differentiation media each comprise the same base media.

In some embodiments, the first media, the first differentiation media, and the second differentiation media each comprise different base media.

In some embodiments, the first differentiation media and the second differentiation media each comprise the same base media, and the first media comprises a base media different from the first and second differentiation media.

In some embodiments, the first differentiation media and the second differentiation media each comprise a base media comprising Iscove's modified dulbecco's medium, bovine serum albumin, recombinant human insulin, human transferrin, and 2-mercaptoethanol.

In some embodiments, the period of time of step (b) is 1-5 days, the period of time of step (c) is 3-15 days, and the period of time of step (d) is 11-25 days.

In some embodiments, steps (a)-(d) occur within 35-45 days.

In some embodiments, the method comprises (e) expanding the NK cells with a maturation media comprising (i) IL-12, IL-15 and IL-18, or (ii) IL-12, IL-2 and IL-18.

In some embodiments, the population of stem cells are induced pluripotent stem cells (iPSCs) or human embryonic stem cells (hESCs).

In some embodiments, the population of hematopoietic progenitors comprises about 30% to about 50% CD34+/CD43+/CD45+ cells.

In some embodiments, the NK cells comprise about 60% to about 100% CD43+/CD45+/CD56+/LFA1+ cells.

In some embodiments, the method comprises expanding the NK cells, wherein the NK cells expand about 10 to about 350 fold.

In some embodiments, the population of stem cells are genetically engineered or edited.

In some embodiments, the NK cells are genetically engineered or edited.

In some embodiments, the yield ratio of NK cells from the population of stem cells is about 2:1 to about 100:1.

In some embodiments, the yield ratio of NK cells from the population of stem cells is about 35:1.

### BRIEF DESCRIPTION OF THE DRAWINGS

FIG. 1 provides a schematic showing an exemplary protocol for differentiating stem cells into hematopoietic progenitors and NK cells.

FIGS. 2A-2D shows characterization of hematopoietic progenitors (HPs) differentiated from stem cells based on the protocol provided in FIG. 1. HPs were characterized at day 15. FIG. 2A shows flow cytometry analysis performed by gating cells to quantify percentage triple-positive for the HP markers CD34/CD43/CD45. FIG. 2B is a graph showing HP purity, ranging from 29-46%, of all cells triple-positive for CD34/CD43/CD45 after culture in either Stemline II, StemSpan SFEM II, or an alternative media. FIG. 2C is a graph showing expansion of HPs at day 15 relative to iPSCs seeded at day 0 for StemSpan SFEM II, Stemline II, or an alternative media. FIG. 2D provides representative brightfield microscope images of the EBs prior to HP harvesting on day 15.

FIGS. 3A-3D show characterization of induced pluripotent stem cell-derived NK (iPSC-NK) cells differentiated from stem cells based on the protocol provided in FIG. 1. FIG. 3A shows flow cytometry analysis performed by gating cells to quantify the percentage of quadruple-positive for four NK markers. CD43/CD45/CD56/LFA1. FIG. 3B is a graph showing NK cell purity using Stemline II, StemSpan SFEM II, or an alternative media. FIG. 3C is a graph showing expansion of iNK at day 40 relative to iPSCs seeded at day 0 for StemSpan SFEM II, Stemline II, or an alternative media. FIG. 3D shows representative brightfield microscope images of the cells at day 39 of iNK differentiation.

FIGS. 4A-D shows the iNK differentiation process yields highly functional iNKs. FIG. 4A shows the percent of CD45+CD5− CD56+ LFA1+ cells in either D40 differentiated NK cells or D47 mature NK cells, with or without LY294002. FIG. 4B shows immunophenotyping of increased purity of NK cells post maturation as well as increases in activation markers NKp46, NKG2D, LFA1, CD16 and decreases in inhibitory markers CD161 and CD73. FIG. 4C and FIG. 4D show D40 differentiated iNKs or D47 matured iNKs incubated with breast adenocarcinoma MDA-MB231 cells expressing a nuclear fluorescent protein at different T:E ratios. iNK cells reduced MDA growth in a dose-responsive manner.

FIGS. 5A-5D show fold expansion results from iPSCs cultured in SFEM II or Stemline media containing various combinations differentiation factors. FIG. 5A shows HP purity of all cells being triple-positive for CD34/CD43/CD45. FIG. 5B shows fold expansion of iPSCs to on day 15, relative to iPSCs seeded at day 0. FIG. 5C shows purity of day 40 iNKs, defined as CD43+/CD45+/CD56+. FIG. 5D shows day 40 iNKs fold expansions of iPSCs to iNKs.

### DETAILED DESCRIPTION

In some aspects, the disclosure provides compositions and methods for generating hematopoietic progenitors, common lymphoid progenitors, pre-NK progenitors, NK progenitors, immature NK cells, and/or NK cells. In some embodiments, the compositions and methods described herein are xenogenic-free.

### Definitions

All publications, patent applications, patents and other references mentioned herein are incorporated by reference in their entirety.

Unless the context indicates otherwise, the various features described herein can be used in any combination with any feature or combination of features set forth herein, and each feature can be excluded or omitted from the combination.

As used herein, the singular forms “a”, “an”, and “the” are include the plural forms as well, unless the context indicates otherwise. The conjugation “and/or” denotes all possible combinations of one or more of listed items.

“Subject” as used herein refers to the recipient of an NK cell population generated by the methods of the disclosure. The term includes mammal, such as primate, mouse, rat, dog, cat, cow, horse, goat, camel, sheep or a pig, preferably a human.

“Treat,” “treating” or “treatment” as used herein refers to any type of action or administration that imparts a benefit to a subject that has a disease or disorder, including improvement in the condition of the patient (i.e., improvement, reduction, or amelioration of one or more symptoms, and partial or complete response to treatment).

The term “effective amount” refers to an amount effective to generate a desired biochemical, cellular, or physiological response. The term “therapeutically effective amount” refer to the amount, dosage, or dosage regime of a therapy effective to cause a desire treatment effect.

“Polynucleotide” as used herein refers to a biopolymer composed of two or more nucleotide monomers covalently bonded through ester linkages between the phosphoryl group of one nucleotide and the hydroxyl group of the sugar component of the next nucleotide in a chain. DNA and RNA are non-limiting examples of polynucleotides.

“Polypeptide” as used herein refers to a polymer consisting of amino acid residues chained together by peptide bonds, forming part of (or the whole of) a protein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The term “variant” means a polynucleotide or polypeptide having at least one substitution, insertion, or deletion in its sequence compared to a reference polynucleotide or polypeptide. A “functional variant” is a variant that retains one or functions of the reference polynucleotide or polypeptide.

The term “inactivating mutation” refers to a mutation in a genomic sequence that disrupts a function of a gene. The inactivating mutation can be in any sequence region (e.g., coding, or non-coding) that contributes to gene expression. Examples include, but are not limited to, cis-acting elements (enhancers) or sequences that are subject to transcription (e.g., mRNA transcript sequences). An inactivating mutation includes mutations that render a gene or its encoded protein non-functional or that reduce the function of the gene or its encoded protein.

As used herein the term “sequence identity”, or “identity” in relation to polynucleotides or polypeptide sequences, refers to the extent to which two optimally aligned polynucleotides or polypeptide sequences match at each position in the alignment across the full length of the reference sequence. The “percent identity” is the number of matched positions in the optimal alignment, divided by length of the reference sequence plus the sum of the lengths of any gaps in the reference sequence in the alignment. The optimal alignment is the alignment that results in the maximum percent identity. Alignment of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.12.0 using default parameters. And, unless noted otherwise, the alignment is an alignment of all or a portion of the polynucleotide or polypeptide sequences of interest across the full length of the reference sequence.

As used herein, the term “engineered” refers to a cell that has been stably transduced with a heterologous polynucleotide or subjected to gene editing to introduce, delete, or modify polynucleotides in the cell, or cells transiently transduced with a polynucleotide in a manner that causes a stable phenotypic change in the cell.

As used herein, the term “stem cell” is used to describe a cell with an undifferentiated phenotype, capable, for example, of differentiating into hematopoietic progenitors, and/or NK cells.

As used herein, the term “pluripotent” means the stem cell is capable of forming substantially all of the differentiated cell types of an organism, at least in culture. For example, embryonic stem cells are a type of pluripotent stem cells that are able to form cells from each of the three germs layers, the ectoderm, the mesoderm, and the endoderm.

As used herein, the terms “induced pluripotent stem cell” and “iPSC” are used to refer to cells, derived from somatic cells, that have been reprogrammed back to a pluripotent state and are capable of proliferation, selectable differentiation, and maturation. iPSCs are stem cells produced from differentiated adult, neonatal, or fetal cells that have been induced or changed, i.e., reprogrammed, into cells capable of differentiating into tissues of all three germ or dermal layers: mesoderm, endoderm, and ectoderm. The iPSCs produced do not refer to cells as they are found in nature.

As used herein, the terms “hematopoietic stem cell”, “hematopoietic progenitor”, or “hematopoietic progenitor cell” refer to stem cells capable of giving rise to both mature myeloid and lymphoid cell types including natural killer cells, T cells, and B cells. Hematopoietic stem cells are typically characterized as CD34+.

The term “progenitor” refers to a cell partially differentiated into a desired cell type. Progenitor cells retain a degree of pluripotency and may differentiate to multiple cell types.

As used herein, “differentiate” or “differentiated” are used to refer to the process and conditions by which undifferentiated, or immature (e.g., unspecialized), cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells.

As used herein, “expand” or “expansion” refer to an increase in the number and/or purity of a cell type within a cell population through mitotic division of cells having limited proliferative capacity, e.g., NK cells.

As used herein, “activity”, “activate”, or “activation” refer to stimulation of activating receptors on a cytotoxic innate lymphoid cell leading to cell division, cytokine secretion (e.g., IFNγ and/or TNFα), and/or release of cytolytic granules to regulate or assist in an immune response.

As used herein, “xenogenic free” refers to compositions and methods lacking animal-derived raw materials (e.g., fetal bovine serum). In some embodiments, the xenogenic free methods described herein do not include feeder cells.

As used herein, “source cells” refers to any progenitor cell known in the art.

As used herein, “yield ratio” refers to a measurement that quantifies the amount of cells produced from a single source cell. For example, a yield ratio of 20:1 NK/iPSC means 20 NK cells were produced from one iPSC. Similarly, if 20,000 NK cells are obtained from an initial population of 1,000 iPSCs, the NK/iPSC yield ratio of NKs from iPSCs would be 20 NKs cells from a single iPSC, or 20:1. In some embodiments, the yield ratio is determined by cell counting. In some embodiments, cell counting is performed at the beginning, throughout, and at the end of the process to calculate how the cells expand overtime based on changes in viable cell density. In some embodiments, dilutions are made to the cells over time (batch feeding).

### Differentiation and Expansion Media

In some embodiments, the disclosure provides media for differentiating stem cells into hematopoietic progenitors. In some embodiments, the disclosure provides media for differentiating hematopoietic progenitors into NK cells. In some embodiments, the disclosure provides media for expanding NK cells. In some embodiments, the differentiating and/or expansion media described herein comprise a serum-free base media with at least one exogenous factor to drive differentiation and/or expansion.

### Xenogenic-Free Media

In some embodiments, in the differentiation and/or expansion media described herein is a defined media. As used herein, “defined media” refers to a growth medium suitable for the in vitro culture of human or animal cells in which all of the chemical components are known. In some embodiments, the differentiation and/or expansion media comprises a base media. In some embodiments, the base media comprises Iscove's Modified Dulbecco's Medium, serum albumin, human insulin, human transferrin, and 2-mercaptoethanol. In some embodiments, the base media comprises human serum albumin. In some embodiments, the base media does not include animal-derived raw materials.

In some embodiments, the base media is selected from StemSpan SFEM II Medium (STEMCELL Technologies; serum-free), Stemline II (Sigma-Aldrich; fully defined, serum- and animal component-free, GMP manufactured), CTS NK Xpander Medium (Gibco; serum-free and animal component-free medium), STEMdiff Hematopoietic—EB Basal Medium (STEMCELL Technologies; serum-free), STEMdiff APEL 2 medium (STEM CELL Technologies; serum-free and animal component-free) or Hematopoietic Progenitor Expansion Medium XF (PromoCell; serum-free and xeno-free medium). In some embodiments, the base media is StemSpan SFEM II media. In some embodiments, the base media is Stemline II media. In some embodiments, the base media is STEMdiff APEL 2 media. In some embodiments, the hematopoietic progenitor differentiation media and the NK cell differentiation media have the same base media. In some embodiments, the hematopoietic progenitor differentiation media and the NK cell differentiation media have different base media.

### Exogenous Factors

In some embodiments, the differentiation and/or expansion media described herein comprises exogenous factors. In some embodiments, the methods of the disclosure comprise contacting different cell populations with various exogenous factors in xenogenic-free media to drive differentiation of cells to e.g., hematopoietic progenitors and/or NK cells. In some embodiments, the exogenous factors include but are not limited to cytokines e.g., interleukins, fibroblast growth factors (FGF), stem cell factor (SCF), Phosphatidylinositol 3-kinases (PI3K) inhibitors, FMS-like tyrosine kinase 3 ligand (FLT3L), Bone morphogenetic protein (BMP) pathway activators, pyrimido-indole derivatives, and aryl hydrocarbon receptor antagonists. In some embodiments, an exogenous factor described herein is commercially available.

In some embodiments, an exogenous factor suitable for use in a differentiation and/or expansion media is a cytokine. Cytokines include interferons, interleukins and growth factors, which are small proteins that play an important role in cell signaling. In some embodiments, the cytokine is an interleukin. In some embodiments, the interleukin is selected from IL-2, IL-7, IL-12, IL-15, IL-18, and any combination thereof. In some embodiments, the cytokine is a growth factor. In some embodiments, the growth factor is selected from a fibroblast growth factor, a vascular endothelial growth factor, and any combination thereof.

In some embodiments, the exogenous factor is interleukin 2 (IL-2). IL-2 is a secreted cytokine produced by activated CD4+ and CD8+T lymphocytes, that is important for the proliferation of T and B lymphocytes. IL-2 is a member of the interleukin 2 (IL2) cytokine subfamily which includes IL-4, IL-7, IL-9, IL-15, IL-21, erythropoietin, and thrombopoietin.

In some embodiments, the exogenous factor is interleukin 7 (IL-7). IL-7 is a member of the interleukin 2 (IL2) cytokine subfamily. Lymphoid differentiation and activation critically depend on IL-7 signaling.

In some embodiments, the exogenous factor is interleukin 12 (IL-12). IL-12 a cytokine that acts on T and natural killer cells, and has a broad array of biological activities. NK cells may acquire memory-like properties following a brief stimulation with IL-12.

In some embodiments, the exogenous factor is interleukin 15 (IL-15). IL-15 is a member of the interleukin 2 (IL2) cytokine subfamily. IL-15 regulates NK cell activation and proliferation.

In some embodiments, the exogenous factor is interleukin 18 (IL-18). IL-18 is a proinflammatory cytokine of the IL-1 family that is constitutively found as a precursor within the cytoplasm of a variety of immune cells. IL-18 has been shown to potently activate NK cells.

In some embodiments, the exogenous factor is Low-density lipoprotein (LDL). LDL induces an increase in proliferation and cytotoxic activity of NK cells.

In some embodiments, the exogenous factor is a fibroblast growth factor (FGF). The FGF family members are cell signaling proteins produced by macrophages. The FGF family comprises 23 members. In some embodiments, the exogenous factor is FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23. In some embodiments, the exogenous factor is FGF2. In some embodiments, the FGF2 is commercially available. In some embodiments, the FGF2 is commercially available from Peprotech. In some embodiments, the FGF2 is commercially available from Gibco.

In some embodiments, the exogenous factor is FMS-like tyrosine kinase 3 ligand (FLT3L). FLT3L is an essential growth factor for NK cells and has been shown to play an important role in the expansion of early hematopoietic progenitors and in the generation of mature peripheral NK cells.

In some embodiments, the exogenous factor is stem cell factor (SCF). SCF plays an important role in the survival of stem cells and the self-renewal and maintenance of stem cells.

In some embodiments, the exogenous factor is a vascular endothelial growth factor (VEGF). The VEGF family is a sub-family of growth factors, the platelet-derived growth factor family of cystine-knot growth factors. The VEGF family comprises five family members. In some embodiments, the exogenous factor is VEGF-A, placenta growth factor (PGF), VEGF-B, VEGF-C and VEGF-D. In some embodiments, the exogenous factor is VEGF-165. VEGF165 is a 38.2 kDa, disulfide-linked homodimeric protein consisting of two 165 amino acid polypeptide chains.

In some embodiments, the exogenous factor is an aryl hydrocarbon inhibitor. The aryl hydrocarbon receptor is a transcription factor that regulates gene expression. The aryl hydrocarbon receptor has roles in regulating immunity, stem cell maintenance, and cellular differentiation. Antagonism of the aryl hydrocarbon receptor has been shown to promote the renewal and expansion of stem cells. In some embodiments, the exogenous factor is an antagonists of the aryl hydrocarbon receptor selected from PD98059, StemRegenin 1 (SR1), GNF351, BAY 2416964, CH-223191, Perillaldehyde, PDM-11, and BAY-218. In some embodiments, the exogenous factor is SR1.

In some embodiments, the exogenous factor is an inhibitor of phosphatidylinositol 3-kinases (PI3Ks). PI3Ks comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane.

Inhibitors of PI3Ks include, but are not limited to, Idelalisib, Copanlisib, Duvelisib, Alpelisib, Umbralisib, Buparlisib, Copanlisib, Dactolisib, Duvelisib, Idelalisib, Leniolisib, Parsaclisib, Paxalisib, Taselisib, Zandelisib, Inavolisib, Apitolisib, Bimiralisib, Eganelisib, Fimepinostat, Gedatolisib, Linperlisib, Nemiralisib, Pictilisib, Pilaralisib, Samotolisib, Seletalisib, Serabelisib, Sonolisib, Tenalisib, Voxtalisib, AMG 319, AZD8186, GSK2636771, SF1126, Acalisib, Omipalisib, AZD8835, CAL263, GSK1059615, MEN1611, PWT33597, TG100-115, ZSTK474, AEZS-136, B591, GNE-477, Hibiscone C, IC87114, LY294002, and PI-103. In some embodiments, the exogenous factor is Idelalisib, Copanlisib, Duvelisib, Alpelisib, Umbralisib, Buparlisib, Copanlisib, Dactolisib, Duvelisib, Idelalisib, Leniolisib, Parsaclisib, Paxalisib, Taselisib, Zandelisib, Inavolisib, Apitolisib, Bimiralisib, Eganelisib, Fimepinostat, Gedatolisib, Linperlisib, Nemiralisib, Pictilisib, Pilaralisib, Samotolisib, Seletalisib, Serabelisib, Sonolisib, Tenalisib, Voxtalisib, AMG 319, AZD8186, GSK2636771, SF1126, Acalisib, Omipalisib, AZD8835, CAL263, GSK1059615, MEN1611, PWT33597, TG100-115, ZSTK474, AEZS-136, B591, GNE-477, Hibiscone C, IC87114, LY294002, PI-103, or any combination thereof. In some embodiments, the exogenous factor is LY294002.

In some embodiments, the exogenous factor is an activator of the BMP pathway. Bone morphogenetic proteins (BMPs) are produced as large precursor molecules which are processed proteolytically to mature peptides after translation. BMPs act through specific transmembrane receptors located on cell surface of the target cells. The BMP receptors are serin-threonine kinases which resemble TGF-β receptors and are divided into two subgroups: type I and type II receptors. BMPs can bind strongly only to the heterotetrametric complex of these receptors. This complex formation is essential to the BMP signal transduction. Inside the target cell, BMP signals are transmitted to the nucleus via specific signal molecules called Smads, which are also responsible for suppression of BMP signals.

BMPs are multifunctional cytokines which are members of the transforming growth factor-beta superfamily. BMP receptors mediate BMP signaling through activating Smad. BMP ligands bind to the BMP receptors BMPRI and BMPRII. Phosphorylated BMPRII activates BMPRI. Phosphorylated BMPRI subsequently phosphorylates receptor-activated Smad proteins (R-Smads), which associate with common mediator-Smad (co-Smad) and enter the nucleus, where they regulate gene expression. BMP pathway activators include those agents disclosed in WO 2014011540, WO 2014062138, and WO 2005117994, which are incorporated herein by reference. BMP pathway activators include, but are not limited to, BMP-5, BMP-6, BMP-7, BMP-8, BMP-2, and BMP-4. In some embodiments, the BMP pathway activator is BMP-4. In some embodiments, the exogenous factor is BMP-4. In some embodiments, the BMP-4 is commercially available. In some embodiments, the BMP-4 is commercially available from Peprotech. In some embodiments, the BMP-4 is commercially available from Invitrogen. In some embodiments, the BMP-4 is commercially available from Biolegend.

In some embodiments, the exogenous factor is an inhibitor of ROCK. Rho associated kinases (ROCK) are serine/threonine kinases that serve downstream effectors of Rho kinases (of which three isoforms exist—RhoA, RhoB and RhoC). ROCK inhibitors include, but are not limited to, polynucleotides, polypeptides, and small molecules. ROCK inhibitors contemplated herein may decrease ROCK expression and/or ROCK activity. Illustrative examples of ROCK inhibitors contemplated herein include, but are not limited to, anti-ROCK antibodies, dominant negative ROCK variants, siRNA, shRNA, miRNA and antisense nucleic acids that target ROCK.

Illustrative ROCK inhibitors contemplated herein include, but are not limited to: thiazovivin, Y27632, Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some embodiments, the ROCK inhibitor is thiazovivin, Y27632, or pyrintegrin. In some embodiments, the ROCK inhibitor is Y27632.

### Mesoderm/Embryoid Body Differentiation Media

In some embodiments, the disclosure provides a differentiation media for generating mesoderm and/or embryoid bodies from stem cells. In some embodiments, mesoderm cells are generated from iPSCs or hESCs. As stem cells begin to differentiate, three distinct germ layers are formed: the ectoderm, mesoderm, and endoderm. Immune cells, such as NK cells, differentiate from mesoderm cells. Embryoid bodies are three-dimensional aggregates that can differentiate into cells of all three germ layers. In some embodiments, the mesoderm cells are produced from embryoid bodies. In some embodiments, the mesoderm cells produced by the compositions and methods of the disclosure are further differentiated to hematopoietic progenitors. In some embodiments, the mesoderm cells produced by the compositions and methods of the disclosure are further differentiated into NK cells.

In some embodiments, a population of stem cells (e.g., iPSCs or hESCs) is cultured with at least one exogenous factor to form mesoderm and/or embryoid body cells. In some embodiments, the exogenous factor is a bone morphogenetic protein (BMP) activator. In some embodiments, the exogenous factor is an FGF. In some embodiments, the exogenous factor is a VEGF. In some embodiments, the exogenous factor is a ROCK inhibitor. In some embodiments, the exogenous factors are selected from a BMP pathway activator, a FGF, a VEGF, a ROCK inhibitor, and any combination thereof. In some embodiments, the exogenous factors comprise a BMP pathway activator and an FGF. In some embodiments, the exogenous factors comprise a BMP pathway activator and a VEGF. In some embodiments, the exogenous factors comprise a BMP pathway activator and a ROCK inhibitor. In some embodiments, the exogenous factors comprise an FGF and a VEGF. In some embodiments, the exogenous factors comprise a FGF and a ROCK inhibitor. In some embodiments, the exogenous factors comprise a VEGF and a ROCK inhibitor. In some embodiments, the exogenous factors comprise a BMP pathway activator, an FGF and a VEGF. In some embodiments, the exogenous factors comprise a BMP pathway activator, a FGF, and a ROCK inhibitor. In some embodiments, the exogenous factors comprise an FGF, a VEGF and a ROCK inhibitor. In some embodiments, the exogenous factors comprise a BMP pathway activator, an FGF, and a ROCK inhibitor. In some embodiments, the exogenous factors comprise a BMP pathway activator, a FGF, a VEGF, and a ROCK inhibitor.

In some embodiments, the exogenous factors comprise BMP4 and FGF2. In some embodiments, the exogenous factors comprise BMP4 and VEGF-165. In some embodiments, the exogenous factors comprise BMP4 and Y27632. In some embodiments, the exogenous factors comprise FGF2 and VEGF-165. In some embodiments, the exogenous factors comprise FGF2 and Y27632. In some embodiments, the exogenous factors comprise VEGF-165 and Y27632. In some embodiments, the exogenous factors comprise BMP4, FGF2 and VEGF-165. In some embodiments, the exogenous factors comprise BMP4, FGF2 and Y27632. In some embodiments, the exogenous factors comprise FGFs, VEGF-165 and Y27632. In some embodiments, the exogenous factors comprise BMP4, FGF2, and Y27632. In some embodiments, the exogenous factors comprise BMP4, FGF2, VEGF-165 and Y27632.

In some embodiments, BMP4, FGF2, VEGF, and/or ROCK inhibitor are used in the mesoderm formation step. For example, the mesoderm formation step may include contacting the cell population with BMP4 and FGF2; with BMP4, FGF2 and a ROCK inhibitor; with BMP4 and VEGF; with BMP4, VEGF and a ROCK inhibitor; with FGF2 and VEGF; with FGF2, VEGF, with a ROCK inhibitor; BMP4, FGF2, and VEGF; BMP4, FGF2, VEGF, and a ROCK inhibitor; or individually any one of BMP4, FGF2, VEGF, and a ROCK inhibitor without the others.

In some embodiments, the bone morphogenetic protein (BMP) activator is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, the bone morphogenetic protein (BMP) activator is present in differentiation media at about 1-50 ng/ml.

In some embodiments, the BMP pathway activator is BMP4. In some embodiments, BMP4 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, BMP4 is present in differentiation media at about 1-50 ng/ml.

In some embodiments, FGF2 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, FGF2 is present in differentiation media at about 1-50 ng/ml.

In some embodiments, VEGF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, VEGF is present in differentiation media at about 5-100 ng/ml.

In some embodiments, the ROCK inhibitor is present in the differentiation media at a concentration of about 0.1-500 μM, about 1-250 μM, about 1-150 IM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 IM, about 14 μM, about 15 μM, about 16 μM, about 17 μM, about 18 μM, about 19 μM, about 20 IM, about 21 μM, about 22 μM, about 23 M about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the ROCK inhibitor is present in differentiation media at about 0.1-20 μM.

In some embodiments, the ROCK inhibitor is Y27632. In some embodiments, Y27632 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 IM, about 6 μM, about 7 μM, about 8 μM, about 9 μM, about 10 PM, about 11 μM, about 12 μM, about 13 μM, about 14 μM, about 15 μM, about 16 μM, about 17 μM, about 18 μM, about 19 μM, about 20 μM, about 21 IM, about 22 μM, about 23 μM, about 24 IM, about 25 μM, about 26 μM, about 27 μM, about 28 μM, about 29 μM, about 30 μM, about 35 M, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, Y27632 is present in differentiation media at about 0.1-100 μM.

In some embodiments, the mesoderm differentiation media comprises a BMP pathway activator, a FGF and a VEGF. In some embodiments, the mesoderm differentiation media comprises a BMP pathway activator, a FGF, a VEGF and a ROCK inhibitor. In some embodiments, the mesoderm differentiation media comprises a defined xenogenic-free base media, a BMP pathway activator, a FGF and a VEGF. In some embodiments, the mesoderm differentiation media comprises a defined xenogenic-free base media, a BMP pathway activator, a FGF, a VEGF, and a ROCK inhibitor. In some embodiments, the mesoderm differentiation media comprise BMP4, FGF2 and VEGF-165. In some embodiments, the mesoderm differentiation media comprises BMP4, FGF, VEGF-165 and a ROCK inhibitor. In some embodiments, the mesoderm differentiation media comprises BMP4, FGF, VEGF-165 and Y27632.

In some embodiments, the mesoderm differentiation media comprise 1-50 ng/mL BMP4, 1-50 ng/mL FGF2 and 1-100 ng/mL VEGF-165. In some embodiments, the mesoderm differentiation media comprises 1-50 ng/mL BMP4, 1-50 ng/mL FGF, 1-100 ng/mL VEGF-165 and 0.1-20 μM of a ROCK inhibitor. In some embodiments, the mesoderm differentiation media comprises 1-50 ng/mL BMP4, 1-50 ng/mL FGF, 1-100 ng/mL VEGF-165 and 0.1-20 μM Y27632.

### Hematopoietic Progenitor Differentiation Media

In some embodiments, the disclosure provides a differentiation media for generating HP cells from mesoderm cells and embryoid body cells. In some embodiments, the mesoderm cells and embryoid body cells produced by the compositions and methods of the disclosure are further differentiated to hematopoietic progenitors.

In some embodiments, a population of HP cells are cultured with at least one exogenous factor to form differentiated NK cells. In some embodiments, the exogenous factor is a BMP pathway activator. In some embodiments, the exogenous factor is exogenous factor is an FGF. In some embodiments, the exogenous factor a VEGF. In some embodiments, the exogenous factor is SCF. In some embodiments, the exogenous factor is TPO. In some embodiments, the exogenous factor is LDL. In some embodiments, the exogenous factor is a PI3K inhibitor. In some embodiments, the exogenous factor is a pyrimido-indole derivative. In some embodiments, the exogenous factor is an aryl hydrocarbon receptor antagonist. In some embodiments, the exogenous factor is a TGF-β receptor inhibitor. In some embodiments, the exogenous factors are selected from a BMP pathway activator, an FGF, a VEGF, SCF, TPO, LDL, a PI3K inhibitor, and any combination thereof. In some embodiments, the exogenous factors are selected from a BMP pathway activator, an FGF, a VEGF, SCF, TPO, LDL, a PI3K inhibitor, a pyrimido-indole derivative, an aryl hydrocarbon receptor antagonist, a TGF-β receptor inhibitor, and any combination thereof.

In some embodiments, the exogenous factors comprise a BMP pathway activator and an FGF. In some embodiments, the exogenous factors comprise a BMP pathway activator and a VEGF. In some embodiments, the exogenous factors comprise a BMP pathway activator and SCF. In some embodiments, the exogenous factors comprise a BMP pathway activator and TPO. In some embodiments, the exogenous factors comprise a BMP pathway activator and LDL. In some embodiments, the exogenous factors comprise a BMP pathway activator and a PI3K inhibitor. In some embodiments, the exogenous factors comprise an FGF and a VEGF. In some embodiments, the exogenous factors comprise an FGF and SCF. In some embodiments, the exogenous factors comprise an FGF and TPO. In some embodiments, the exogenous factors comprise an FGF and LDL. In some embodiments, the exogenous factors comprise an FGF and a PI3K inhibitor. In some embodiments, the exogenous factors comprise a VEGF and SCF. In some embodiments, the exogenous factors comprise a VEGF and TPO. In some embodiments, the exogenous factors comprise a VEGF and LDL. In some embodiments, the exogenous factors comprise a VEGF and a PI3K inhibitor. In some embodiments, the exogenous factors comprise SCF and TPO. In some embodiments, the exogenous factors comprise SCF and LDL. In some embodiments, the exogenous factors comprise SCF and a PI3K inhibitor. In some embodiments, the exogenous factors comprise TPO and LDL. In some embodiments, the exogenous factors comprise TPO and a PI3K inhibitor. In some embodiments, the exogenous factors comprise LDL and a PI3K inhibitor.

In some embodiments, the exogenous factors comprise a BMP pathway activator, an FGF, and a VEGF. In some embodiments, the exogenous factors comprise a BMP pathway activator, an FGF, and SCF. In some embodiments, the exogenous factors comprise a BMP pathway activator, an FGF, and TPO. In some embodiments, the exogenous factors comprise a BMP pathway activator, an FGF, and LDL. In some embodiments, the exogenous factors comprise a BMP pathway activator, an FGF, and a PI3K inhibitor. In some embodiments, the exogenous factors comprise a BMP pathway activator, a VEGF, and SCF. In some embodiments, the exogenous factors comprise a BMP pathway activator, a VEGF, and TPO. In some embodiments, the exogenous factors comprise a BMP pathway activator, a VEGF, and LDL. In some embodiments, the exogenous factors comprise a BMP pathway activator, a VEGF, and a PI3K inhibitor. In some embodiments, the exogenous factors comprise a BMP pathway activator, SCF, and TPO. In some embodiments, the exogenous factors comprise a BMP pathway activator, SCF, and LDL. In some embodiments, the exogenous factors comprise a BMP pathway activator, SCF, and a PI3K inhibitor. In some embodiments, the exogenous factors comprise a BMP pathway activator, TPO, and LDL. In some embodiments, the exogenous factors comprise a BMP pathway activator, TPO, and a PI3K inhibitor. In some embodiments, the exogenous factors comprise a BMP pathway activator, LDL, and a PI3K inhibitor. In some embodiments, the exogenous factors comprise an FGF, a VEGF, and SCF. In some embodiments, the exogenous factors comprise an FGF, a VEGF, and TPO. In some embodiments, the exogenous factors comprise an FGF, a VEGF, and LDL. In some embodiments, the exogenous factors comprise an FGF, a VEGF, and LDL. In some embodiments, the exogenous factors comprise an FGF, SCF, and TPO. In some embodiments, the exogenous factors comprise an FGF, SCF, and LDL. In some embodiments, the exogenous factors comprise an FGF, SCF, and a PI3K inhibitor. In some embodiments, the exogenous factors comprise an FGF, TPO, and LDL. In some embodiments, the exogenous factors comprise an FGF, TPO, and a PI3K inhibitor. In some embodiments, the exogenous factors comprise an FGF, LDL, and a PI3K inhibitor. In some embodiments, the exogenous factors comprise a VEGF, SCF, and TPO. In some embodiments, the exogenous factors comprise a VEGF, SCF, and LDL. In some embodiments, the exogenous factors comprise a VEGF, SCF, and a PI3K inhibitor. In some embodiments, the exogenous factors comprise a VEGF, TPO, and LDL. In some embodiments, the exogenous factors comprise a VEGF, TPO, and a PI3K inhibitor.

In some embodiments, the exogenous factors comprise a BMP pathway activator, an FGF, a VEGF, and SCF. In some embodiments, the exogenous factors comprise a BMP pathway activator, an FGF, a VEGF, and TPO. In some embodiments, the exogenous factors comprise a BMP pathway activator, an FGF, a VEGF, and LDL. In some embodiments, the exogenous factors comprise a BMP pathway activator, an FGF, a VEGF, and a PI3K inhibitor. In some embodiments, the exogenous factors comprise a BMP pathway activator, an FGF, SCF, and TPO. In some embodiments, the exogenous factors comprise a BMP pathway activator, an FGF, SCF, and LDL. In some embodiments, the exogenous factors comprise a BMP pathway activator, an FGF, SCF, and a PI3K inhibitor. In some embodiments, the exogenous factors comprise a BMP pathway activator, an FGF, TPO, and LDL. In some embodiments, the exogenous factors comprise a BMP pathway activator, an FGF, TPO, and a PI3K inhibitor. In some embodiments, the exogenous factors comprise a BMP pathway activator, an FGF, LDL, and a PI3K inhibitor. In some embodiments, the exogenous factors comprise a BMP pathway activator, a VEGF, SCF, and TPO. In some embodiments, the exogenous factors comprise a BMP pathway activator, a VEGF, SCF, and LDL. In some embodiments, the exogenous factors comprise a BMP pathway activator, a VEGF, SCF, and a PI3K inhibitor. In some embodiments, the exogenous factors comprise a BMP pathway activator, a VEGF, TPO, and LDL. In some embodiments, the exogenous factors comprise a BMP pathway activator, a VEGF, TPO, and a PI3K inhibitor. In some embodiments, the exogenous factors comprise a BMP pathway activator, a VEGF, LDL, and a PI3K inhibitor. In some embodiments, the exogenous factors comprise a BMP pathway activator, SCF, TPO, and LDL. In some embodiments, the exogenous factors comprise a BMP pathway activator, SCF, TPO, and a PI3K inhibitor. In some embodiments, the exogenous factors comprise a BMP pathway activator, SCF, TPO, and a PI3K inhibitor. In some embodiments, the exogenous factors comprise a BMP pathway activator, TPO, LDL, and a PI3K inhibitor. In some embodiments, the exogenous factors comprise an FGF, a VEGF, SCF, and TPO. In some embodiments, the exogenous factors comprise an FGF, a VEGF, SCF, and LDL. In some embodiments, the exogenous factors comprise an FGF, a VEGF, SCF, and a PI3K inhibitor. In some embodiments, the exogenous factors comprise an FGF, a VEGF, TPO, and LDL. In some embodiments, the exogenous factors comprise an FGF, a VEGF, TPO, and a PI3K inhibitor. In some embodiments, the exogenous factors comprise an FGF, a VEGF, LDL, and a PI3K inhibitor. In some embodiments, the exogenous factors comprise an FGF, SCF, TPO, and LDL. In some embodiments, the exogenous factors comprise an FGF, SCF, TPO, and a PI3K inhibitor. In some embodiments, the exogenous factors comprise an FGF, SCF, LDL, and a PI3K inhibitor. In some embodiments, the exogenous factors comprise an FGF, TPO, LDL, and a PI3K inhibitor.

In some embodiments, the exogenous factors comprise a BMP pathway activator, an FGF, a VEGF, SCF, and TPO. In some embodiments, the exogenous factors comprise a BMP pathway activator, an FGF, a VEGF, SCF, and LDL. In some embodiments, the exogenous factors comprise a BMP pathway activator, an FGF, a VEGF, SCF, and a PI3K inhibitor. In some embodiments, the exogenous factors comprise a BMP pathway activator, an FGF, a VEGF, TPO, and LDL. In some embodiments, the exogenous factors comprise a BMP pathway activator, an FGF, a VEGF, TPO, and a PI3K inhibitor. In some embodiments, the exogenous factors comprise a BMP pathway activator, an FGF, a VEGF, LDL, and a PI3K inhibitor. In some embodiments, the exogenous factors comprise a BMP pathway activator, an FGF, SCF, TPO, and LDL. In some embodiments, the exogenous factors comprise a BMP pathway activator, an FGF, SCF, TPO, and a PI3K inhibitor. In some embodiments, the exogenous factors comprise a BMP pathway activator, an FGF, SCF, LDL, and a PI3K inhibitor. In some embodiments, the exogenous factors comprise a BMP pathway activator, an FGF, TPO, LDL, and a PI3K inhibitor. In some embodiments, the exogenous factors comprise a BMP pathway activator, a VEGF, SCF, TPO, and LDL. In some embodiments, the exogenous factors comprise a BMP pathway activator, a VEGF, SCF, TPO, and a PI3K inhibitor. In some embodiments, the exogenous factors comprise a BMP pathway activator, a VEGF, SCF, LDL, and a PI3K inhibitor. In some embodiments, the exogenous factors comprise a BMP pathway activator, a VEGF, TPO, LDL, and a PI3K inhibitor. In some embodiments, the exogenous factors comprise a BMP pathway activator, SCF, TPO, LDL, and a PI3K inhibitor. In some embodiments, the exogenous factors comprise an FGF, a VEGF, SCF, TPO, and LDL. In some embodiments, the exogenous factors comprise an FGF, a VEGF, SCF, TPO, and a PI3K inhibitor. In some embodiments, the exogenous factors comprise an FGF, a VEGF, SCF, LDL, and a PI3K inhibitor. In some embodiments, the exogenous factors comprise an FGF, a VEGF, TPO, LDL, and a PI3K inhibitor. In some embodiments, the exogenous factors comprise an FGF, a VEGF, SCF, TPO, and LDL. In some embodiments, the exogenous factors comprise an FGF, a VEGF, SCF, TPO, and a PI3K inhibitor. In some embodiments, the exogenous factors comprise an FGF, a VEGF, SCF, LDL, and a PI3K inhibitor. In some embodiments, the exogenous factors comprise an FGF, a VEGF, TPO, LDL, and a PI3K inhibitor. In some embodiments, the exogenous factors comprise an FGF, SCF, TPO, LDL, and a PI3K inhibitor. In some embodiments, the exogenous factors comprise a VEGF, SCF, TPO, LDL, and a PI3K inhibitor.

In some embodiments, the exogenous factors comprise a BMP pathway activator, an FGF, a VEGF, SCF, TPO, and LDL. In some embodiments, the exogenous factors comprise a BMP pathway activator, an FGF, a VEGF, SCF, TPO, and a PI3K inhibitor. In some embodiments, the exogenous factors comprise a BMP pathway activator, an FGF, a VEGF, SCF, LDL, and a PI3K inhibitor. In some embodiments, the exogenous factors comprise a BMP pathway activator, an FGF, a VEGF, TPO, LDL, and a PI3K inhibitor. In some embodiments, the exogenous factors comprise a BMP pathway activator, an FGF, SCF, TPO, LDL, and a PI3K inhibitor. In some embodiments, the exogenous factors comprise a BMP pathway activator, a VEGF, SCF, TPO, LDL, and a PI3K inhibitor. In some embodiments, the exogenous factors comprise an FGF, a VEGF, SCF, TPO, LDL, and a PI3K inhibitor.

In some embodiments, the exogenous factors comprise an BMP pathway activator, FGF, a VEGF, SCF, TPO, LDL, and a PI3K inhibitor.

In some embodiments, the exogenous factors comprise BMP4 and FGF2. In some embodiments, the exogenous factors comprise BMP4 and VEGF-165. In some embodiments, the exogenous factors comprise BMP4 and SCF. In some embodiments, the exogenous factors comprise BMP4 and TPO. In some embodiments, the exogenous factors comprise BMP4 and LDL. In some embodiments, the exogenous factors comprise BMP4 and LY294002. In some embodiments, the exogenous factors comprise FGF2 and VEGF-165. In some embodiments, the exogenous factors comprise FGF2 and SCF. In some embodiments, the exogenous factors comprise FGF2 and TPO. In some embodiments, the exogenous factors comprise FGF2 and LDL. In some embodiments, the exogenous factors comprise FGF2 and LY294002. In some embodiments, the exogenous factors comprise VEGF-165 and SCF. In some embodiments, the exogenous factors comprise VEGF-165 and TPO. In some embodiments, the exogenous factors comprise VEGF-165 and LDL. In some embodiments, the exogenous factors comprise VEGF-165 and LY294002. In some embodiments, the exogenous factors comprise SCF and TPO. In some embodiments, the exogenous factors comprise SCF and LDL. In some embodiments, the exogenous factors comprise SCF and LY294002. In some embodiments, the exogenous factors comprise TPO and LDL. In some embodiments, the exogenous factors comprise TPO and LY294002. In some embodiments, the exogenous factors comprise LDL and LY294002.

In some embodiments, the exogenous factors comprise BMP4, FGF2, and VEGF-165. In some embodiments, the exogenous factors comprise BMP4, FGF2, and SCF. In some embodiments, the exogenous factors comprise BMP4, FGF2, and TPO. In some embodiments, the exogenous factors comprise BMP4, FGF2, and LDL. In some embodiments, the exogenous factors comprise BMP4, FGF2, and LY294002. In some embodiments, the exogenous factors comprise BMP4, VEGF-165, and SCF. In some embodiments, the exogenous factors comprise BMP4, VEGF-165, and TPO. In some embodiments, the exogenous factors comprise BMP4, VEGF-165, and LDL. In some embodiments, the exogenous factors comprise BMP4, VEGF-165, and LY294002. In some embodiments, the exogenous factors comprise BMP4, SCF, and TPO. In some embodiments, the exogenous factors comprise BMP4, SCF, and LDL. In some embodiments, the exogenous factors comprise BMP4, SCF, and LY294002. In some embodiments, the exogenous factors comprise BMP4, TPO, and LDL. In some embodiments, the exogenous factors comprise BMP4, TPO, and LY294002. In some embodiments, the exogenous factors comprise BMP4, LDL, and LY294002. In some embodiments, the exogenous factors comprise FGF2, VEGF-165, and SCF. In some embodiments, the exogenous factors comprise FGF2, VEGF-165, and TPO. In some embodiments, the exogenous factors comprise FGF2, VEGF-165, and LDL. In some embodiments, the exogenous factors comprise FGF2, VEGF-165, and LDL. In some embodiments, the exogenous factors comprise FGF2, SCF, and TPO. In some embodiments, the exogenous factors comprise FGF2, SCF, and LDL. In some embodiments, the exogenous factors comprise FGF2, SCF, and LY294002. In some embodiments, the exogenous factors comprise FGF2, TPO, and LDL. In some embodiments, the exogenous factors comprise FGF2, TPO, and LY294002. In some embodiments, the exogenous factors comprise FGF2, LDL, and LY294002. In some embodiments, the exogenous factors comprise VEGF-165, SCF, and TPO. In some embodiments, the exogenous factors comprise VEGF-165, SCF, and LDL. In some embodiments, the exogenous factors comprise VEGF-165, SCF, and LY294002. In some embodiments, the exogenous factors comprise VEGF-165, TPO, and LDL. In some embodiments, the exogenous factors comprise VEGF-165, TPO, and LY294002.

In some embodiments, the exogenous factors comprise BMP4, FGF2, VEGF-165, and SCF. In some embodiments, the exogenous factors comprise BMP4, FGF2, VEGF-165, and TPO. In some embodiments, the exogenous factors comprise BMP4, FGF2, VEGF-165, and LDL. In some embodiments, the exogenous factors comprise BMP4, FGF2, VEGF-165, and LY294002. In some embodiments, the exogenous factors comprise BMP4, FGF2, SCF, and TPO. In some embodiments, the exogenous factors comprise BMP4, FGF2, SCF, and LDL. In some embodiments, the exogenous factors comprise BMP4, FGF2, SCF, and LY294002. In some embodiments, the exogenous factors comprise BMP4, FGF2, TPO, and LDL. In some embodiments, the exogenous factors comprise BMP4, FGF2, TPO, and LY294002. In some embodiments, the exogenous factors comprise BMP4, FGF2, LDL, and LY294002. In some embodiments, the exogenous factors comprise BMP4, VEGF-165, SCF, and TPO. In some embodiments, the exogenous factors comprise BMP4, VEGF-165, SCF, and LDL. In some embodiments, the exogenous factors comprise BMP4, VEGF-165, SCF, and LY294002. In some embodiments, the exogenous factors comprise BMP4, VEGF-165, TPO, and LDL In some embodiments, the exogenous factors comprise BMP4, VEGF-165, TPO, and LY294002. In some embodiments, the exogenous factors comprise BMP4, VEGF-165, LDL, and LY294002. In some embodiments, the exogenous factors comprise BMP4, SCF, TPO, and LDL. In some embodiments, the exogenous factors comprise BMP4, SCF, TPO, and LY294002. In some embodiments, the exogenous factors comprise BMP4, SCF, TPO, and LY294002. In some embodiments, the exogenous factors comprise BMP4, TPO, LDL, and LY294002. In some embodiments, the exogenous factors comprise FGF2, VEGF-165, SCF, and TPO. In some embodiments, the exogenous factors comprise FGF2, VEGF-165, SCF, and LDL. In some embodiments, the exogenous factors comprise FGF2, VEGF-165, SCF, and LY294002. In some embodiments, the exogenous factors comprise FGF2, VEGF-165, TPO, and LDL. In some embodiments, the exogenous factors comprise FGF2, VEGF-165, TPO, and LY294002. In some embodiments, the exogenous factors comprise FGF2, VEGF-165, LDL, and LY294002. In some embodiments, the exogenous factors comprise FGF2, SCF, TPO, and LDL. In some embodiments, the exogenous factors comprise FGF2, SCF, TPO, and LY294002. In some embodiments, the exogenous factors comprise FGF2, SCF, LDL, and LY294002. In some embodiments, the exogenous factors comprise FGF2, TPO, LDL, and LY294002.

In some embodiments, the exogenous factors comprise BMP4, FGF2, VEGF-165, SCF, and TPO. In some embodiments, the exogenous factors comprise BMP4, FGF2, VEGF-165, SCF, and LDL. In some embodiments, the exogenous factors comprise BMP4, FGF2, VEGF-165, SCF, and LY294002. In some embodiments, the exogenous factors comprise BMP4, FGF2, VEGF-165, TPO, and LDL. In some embodiments, the exogenous factors comprise BMP4, FGF2, VEGF-165, TPO, and LY294002. In some embodiments, the exogenous factors comprise BMP4, FGF2, VEGF-165, LDL, and LY294002. In some embodiments, the exogenous factors comprise BMP4, FGF2, SCF, TPO, and LDL. In some embodiments, the exogenous factors comprise BMP4, FGF2, SCF, TPO, and LY294002. In some embodiments, the exogenous factors comprise BMP4, FGF2, SCF, LDL, and LY294002. In some embodiments, the exogenous factors comprise BMP4, FGF2, TPO, LDL, and LY294002. In some embodiments, the exogenous factors comprise BMP4, VEGF-165, SCF, TPO, and LDL. In some embodiments, the exogenous factors comprise BMP4, VEGF-165, SCF, TPO, and LY294002. In some embodiments, the exogenous factors comprise BMP4, VEGF-165, SCF, LDL, and LY294002. In some embodiments, the exogenous factors comprise BMP4, VEGF-165, TPO, LDL, and LY294002. In some embodiments, the exogenous factors comprise BMP4, SCF, TPO, LDL, and LY294002. In some embodiments, the exogenous factors comprise FGF2, VEGF-165, SCF, TPO, and LDL. In some embodiments, the exogenous factors comprise FGF2, VEGF-165, SCF, TPO, and LY294002. In some embodiments, the exogenous factors comprise FGF2, VEGF-165, SCF, LDL, and LY294002. In some embodiments, the exogenous factors comprise FGF2, VEGF-165, TPO, LDL, and LY294002. In some embodiments, the exogenous factors comprise FGF2, VEGF-165, SCF, TPO, and LDL. In some embodiments, the exogenous factors comprise FGF2, VEGF-165, SCF, TPO, and LY294002. In some embodiments, the exogenous factors comprise FGF2, VEGF-165, SCF, LDL, and LY294002. In some embodiments, the exogenous factors comprise FGF2, VEGF-165, TPO, LDL, and LY294002. In some embodiments, the exogenous factors comprise FGF2, SCF, TPO, LDL, and LY294002. In some embodiments, the exogenous factors comprise VEGF-165, SCF, TPO, LDL, and LY294002.

In some embodiments, the exogenous factors comprise BMP4, FGF2, VEGF-165, SCF, TPO, and LDL. In some embodiments, the exogenous factors comprise BMP4, FGF2, VEGF-165, SCF, TPO, and LY294002. In some embodiments, the exogenous factors comprise BMP4, FGF2, VEGF-165, SCF, LDL, and LY294002. In some embodiments, the exogenous factors comprise BMP4, FGF2, VEGF-165, TPO, LDL, and LY294002. In some embodiments, the exogenous factors comprise BMP4, FGF2, SCF, TPO, LDL, and LY294002. In some embodiments, the exogenous factors comprise BMP4, VEGF-165, SCF, TPO, LDL, and LY294002. In some embodiments, the exogenous factors comprise FGF2, VEGF-165, SCF, TPO, LDL, and LY294002.

In some embodiments, the exogenous factors comprise BMP4, FGF2, VEGF-165, SCF, TPO, LDL, and LY294002.

In some embodiments, the bone morphogenetic protein (BMP) activator is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, the bone morphogenetic protein (BMP) activator is present in differentiation media at about 1-50 ng/ml.

In some embodiments, the bone morphogenetic protein (BMP) activator is BMP4. In some embodiments, BMP4 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, BMP4 is present in differentiation media at about 1-50 ng/ml.

In some embodiments, FGF2 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, FGF2 is present in differentiation media at about 1-50 ng/ml.

In some embodiments, VEGF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, VEGF is present in differentiation media at about 1-100 ng/ml.

In some embodiments, SCF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, SCF is present in differentiation media at about 1-100 ng/ml.

In some embodiments, TPO is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, TPO is present in differentiation media at about 1-100 ng/ml.

In some embodiments, LDL is present in the differentiation media at a concentration of about 0.1-500 μg/ml, about 1-250 μg/ml, about 1-150 μg/ml, about 5-100 μg/ml, about or about 0.1 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, about 20 μg/ml, about 21 μg/ml, about 22 μg/ml, about 23 μg/ml, about 24 μg/ml, about 25 μg/ml, about 26 μg/ml, about 27 μg/ml, about 28 μg/ml, about 29 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 sg/ml, about or 100 μg/ml, or any range derivable therein. In some embodiments, LDL is present in differentiation media at about 1-50 μg/ml.

In some embodiments, the PI3K inhibitor is present in the differentiation media at a concentration of about 0.1-500 μM, about 1-250 μM, about 1-150 μM, about 5-100 PM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 M, about 27 μM, about 28 μM, about 29 μM, about 30 μM, about 35 μM, about 40 μM, about 45 μM, about 50 μM, about 55 μM, about 60 μM, about 65 μM, about 70 PM, about 75 μM, about 80 μM, about 85 μM, about 90 μM, about 95 μM, about or 100 μM, or any range derivable therein. In some embodiments, the PI3K inhibitor is present in differentiation media at about 0.1-100 μM.

In some embodiments, the PI3K inhibitor is LY294002. In some embodiments, LY294002 is present at a concentration of about 0.1-500 μM, about 1-250 μM, about 1-150 M, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 IM, about 35 μM, about 40 μM, about 45 μM, about 50 μM, about 55 μM, about 60 μM, about 65 M, about 70 μM, about 75 μM, about 80 μM, about 85 μM, about 90 μM, about 95 μM, about or 100 μM, about or 100 μM, or any range derivable therein. In some embodiments, LY294002 is present in differentiation media at about 0.1-100 μM.

In some embodiments, the pyrimido-indole derivative is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 PM, about 65 μM, about 70 μM, about 75 μM, about 80 μM, about 85 μM, about 90 μM, about 95 μM, about or 100 μM, or any range derivable therein. In some embodiments, the pyrimido-indole derivative is present in differentiation media at about 0.1-10 μM.

In some embodiments, the pyrimido-indole derivative is UM729. In some embodiments, UM729 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 IM, about 35 IM, about 40 μM, about 45 μM, about 50 IM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, UM729 is present in differentiation media at about 0.1-10 μM.

In some embodiments, the aryl hydrocarbon receptor antagonist is present in the differentiation media at a concentration of about 0.1-500 PM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 IM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the aryl hydrocarbon receptor antagonist is present in differentiation media at about 0.1-10 μM.

In some embodiments, the aryl hydrocarbon receptor antagonist is StemRegenin 1 (SR1). In some embodiments, SR1 is present at a concentration of about 0.1-500 PM, about 1-250 μM, about 1-150 μM, about 5-100 μM, about or about 0.1 μM, about 1 μM, about 2 μM, about 3 μM, about 4 μM, about 5 IM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 IM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, SR1 is present in differentiation media at about 0.1-10 PM.

In some embodiments, a TGF-β receptor inhibitor is present in the differentiation media at a concentration of about 0.1-500 μM, about 1-250 IM, about 1-150 JIM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 IM, about 70 μM, about 75 μM, about 80 μM, about 85 μM, about 90 μM, about 95 μM, about or 100 μM, or any range derivable therein. In some embodiments, the TGF-β receptor inhibitor is present in differentiation media at about 0.1-20 μM.

In some embodiments, the TGF-β receptor inhibitor is GW788388. In some embodiments, GW788388 is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 PM, about 75 μM, about 80 μM, about 85 μM, about 90 μM, about 95 μM, about or 100 μM, or any range derivable therein. In some embodiments, GW788388 is present in differentiation media at about 0.1-20 μM.

In some embodiments, the TGF-β receptor inhibitor is SB431542. In some embodiments, GW788388 is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 IM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, SB431542 is present in differentiation media at about 0.1-20 μM.

In some embodiments, the HP differentiation media comprises a BMP pathway activator, a FGF and a VEGF. In some embodiments, the HP differentiation media comprises a BMP pathway activator, a FGF, a VEGF and a ROCK inhibitor. In some embodiments, the HP differentiation media comprise BMP4, FGF2 and VEGF-165. In some embodiments, the HP differentiation media comprises BMP4, FGF, VEGF-165 and a ROCK inhibitor. In some embodiments, the HP differentiation media comprises BMP4, FGF, VEGF-165 and Y27632.

In some embodiments, the HP differentiation media comprise 1-50 ng/mL BMP4, 5-50 ng/mL FGF2 and 1-100 ng/mL VEGF-165. In some embodiments, the HP differentiation media comprises 1-50 ng/mL BMP4, 1-50 ng/mL FGF, 1-100 ng/mL VEGF-165 and 1-20 μM of a ROCK inhibitor. In some embodiments, the HP differentiation media comprises 1-50 ng/mL BMP4, 1-50 ng/mL FGF, 1-100 ng/mL VEGF-165 and 1-20 μM Y27632.

In some embodiments, the HP differentiation media comprises a BMP pathway activator, a FGF, a VEGF, and a pyrimido-indole derivative. In some embodiments, the HP differentiation media comprises a BMP pathway activator, a FGF, a VEGF, and an aryl hydrocarbon receptor antagonist. In some embodiments, the HP differentiation media comprises a BMP pathway activator, a FGF, a VEGF, a pyrimido-indole derivative, and an aryl hydrocarbon receptor antagonist.

In some embodiments, the HP differentiation media comprises BMP4, FGF, VEGF-165, and a pyrimido-indole derivative. In some embodiments, the HP differentiation media comprises BMP4, FGF, VEGF-165, and an aryl hydrocarbon receptor antagonist. In some embodiments, the HP differentiation media comprises BMP4, FGF, VEGF-165, a pyrimido-indole derivative, and an aryl hydrocarbon receptor antagonist. In some embodiments, the HP differentiation media comprises BMP4, FGF, VEGF-165, and SR1. In some embodiments, the HP differentiation media comprises BMP4, FGF, VEGF-165, and UM729. In some embodiments, the HP differentiation media comprises BMP4, FGF, VEGF-165, UM729, and SR1.

In some embodiments, the HP differentiation media comprises 1-50 ng/mL BMP4, 1-50 ng/mL FGF, 1-100 ng/mL VEGF-165, and 0.1-10 μM UM729. In some embodiments, the HP differentiation media comprises 1-50 ng/mL BMP4, 1-50 ng/mL FGF, 1-100 ng/mL VEGF-165, and 0.1-10 μM SR1. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1.

In some embodiments, the HP differentiation media comprises a BMP pathway activator, a FGF, a VEGF, and a TGF-β receptor inhibitor. In some embodiments, the HP differentiation media comprises a BMP pathway activator, a FGF, a VEGF, a pyrimido-indole derivative, and a TGF-β receptor inhibitor. In some embodiments, the HP differentiation media comprises a BMP pathway activator, a FGF, a VEGF, an aryl hydrocarbon receptor antagonist, and a TGF-β receptor inhibitor. In some embodiments, the HP differentiation media comprises a BMP pathway activator, a FGF, a VEGF, a pyrimido-indole derivative, an aryl hydrocarbon receptor antagonist, and a TGF-β receptor inhibitor.

In some embodiments, the HP differentiation media comprises BMP4, FGF, VEGF-165, and a TGF-β receptor inhibitor. In some embodiments, the HP differentiation media comprises BMP4, FGF, VEGF-165, and GW788388. In some embodiments, the HP differentiation media comprises BMP4, FGF, VEGF-165, UM729, and GW788388. In some embodiments, the HP differentiation media comprises BMP4, FGF, VEGF-165, SR1, and GW788388. In some embodiments, the HP differentiation media comprises BMP4, FGF, VEGF-165, UM729, SR1, and GW788388.

In some embodiments, the HP differentiation media comprises 1-50 ng/mL BMP4, 1-50 ng/mL FGF, 1-100 ng/mL VEGF-165, and 0.1-20 μM of a TGF-β receptor inhibitor. In some embodiments, the HP differentiation media comprises 1-50 ng/mL BMP4, 1-50 ng/mL FGF, 1-100 ng/mL VEGF-165, and 0.1-20 μM GW788388. In some embodiments, the HP differentiation media comprises 1-50 ng/mL BMP4, 1-50 ng/mL FGF, 1-100 ng/mL VEGF-165, 0.1-10 μM UM729, and 0.1-20 μM GW788388. In some embodiments, the HP differentiation media comprises 1-50 ng/mL BMP4, 1-50 ng/mL FGF, 1-100 ng/mL VEGF-165, 0.1-10 μM SR1, and 0.1-20 μM GW788388. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1, and 0.1-20 μM GW788388.

In some embodiments, the HP differentiation media comprises BMP4, FGF, VEGF-165, and a TGF-β receptor inhibitor. In some embodiments, the HP differentiation media comprises BMP4, FGF, VEGF-165, and SB431542. In some embodiments, the HP differentiation media comprises BMP4, FGF, VEGF-165, UM729, and SB431542. In some embodiments, the HP differentiation media comprises BMP4, FGF, VEGF-165, SR1, and SB431542. In some embodiments, the HP differentiation media comprises BMP4, FGF, VEGF-165, UM729, SR1, and SB431542.

In some embodiments, the HP differentiation media comprises 1-50 ng/mL BMP4, 1-50 ng/mL FGF, 1-100 ng/mL VEGF-165, and 0.1-20 μM of a TGF-β receptor inhibitor. In some embodiments, the HP differentiation media comprises 1-50 ng/mL BMP4, 1-50 ng/mL FGF, 1-100 ng/mL VEGF-165, and 0.1-20 μM SB431542. In some embodiments, the HP differentiation media comprises 1-50 ng/mL BMP4, 1-50 ng/mL FGF, 1-100 ng/mL VEGF-165, 0.1-10 μM UM729, and 0.1-20 μM SB431542. In some embodiments, the HP differentiation media comprises 1-50 ng/mL BMP4, 1-50 ng/mL FGF, 1-100 ng/mL VEGF-165, 0.1-10 μM SR1, and 0.1-20 μM SB431542. In some embodiments, the HP differentiation media comprises 1-50 ng/mL BMP4, 1-50 ng/mL FGF, 1-100 ng/mL VEGF-165, 0.1-10 μM UM729, and 0.1-10 μM SR1, and 0.1-20 μM SB431542.

### NK Cell Differentiation Media

In some embodiments, the disclosure provides a differentiation media for generating NK cells from HP cells. In some embodiments, NK cells are generated from HP cells. In some embodiments, the HP cells produced by the compositions and methods of the disclosure are further differentiated to NK cells.

In some embodiments, a population of HP cells are cultured with at least one exogenous factor to form differentiated NK cells. In some embodiments, the exogenous factor is stem cell factor (SCF). In some embodiments, the exogenous factor is IL-7. In some embodiments, the exogenous factor is IL-15. In some embodiments, the exogenous factor is IL-12. In some embodiments, the exogenous factor is FLT3L. In some embodiments, the exogenous factor is a pyrimido-indole derivative. In some embodiments, the exogenous factor is an aryl hydrocarbon receptor antagonist. In some embodiments, the exogenous factors are selected from SCF, IL-7, IL-15, IL-12, a pyrimido-indole derivative, and an aryl hydrocarbon receptor antagonist.

In some embodiments, the exogenous factors comprise SCF and IL-7. In some embodiments, the exogenous factors comprise SCF and IL-15. In some embodiments, the exogenous factors comprise SCF and IL-12. In some embodiments, the exogenous factors comprise SCF and FLT3L. In some embodiments, the exogenous factors comprise SCF and pyrimido-indole derivative. In some embodiments, the exogenous factors comprise SCF and an aryl hydrocarbon receptor antagonist. In some embodiments, the exogenous factors comprise an IL-7 and an IL-15. In some embodiments, the exogenous factors comprise IL-7 and IL-12. In some embodiments, the exogenous factors comprise IL-7 and FLT3L. In some embodiments, the exogenous factors comprise IL-7 and pyrimido-indole derivative. In some embodiments, the exogenous factors comprise IL-7 and an aryl hydrocarbon receptor antagonist. In some embodiments, the exogenous factors comprise IL-15 and IL-12. In some embodiments, the exogenous factors comprise IL-15 and FLT3L. In some embodiments, the exogenous factors comprise IL-15 and pyrimido-indole derivative. In some embodiments, the exogenous factors comprise IL-15 and an aryl hydrocarbon receptor antagonist. In some embodiments, the exogenous factors comprise IL-12 and FLT3L. In some embodiments, the exogenous factors comprise IL-12 and pyrimido-indole derivative. In some embodiments, the exogenous factors comprise IL-12 and an aryl hydrocarbon receptor antagonist. In some embodiments, the exogenous factors comprise FLT3L and pyrimido-indole derivative. In some embodiments, the exogenous factors comprise FLT3L and an aryl hydrocarbon receptor antagonist. In some embodiments, the exogenous factors comprise a pyrimido-indole derivative and an aryl hydrocarbon receptor antagonist.

In some embodiments, the exogenous factors comprise SCF, IL-7, and IL-12. In some embodiments, the exogenous factors comprise SCF, IL-7, and IL-15. In some embodiments, the exogenous factors comprise SCF, IL-7, and FLT3L. In some embodiments, the exogenous factors comprise SCF, IL-7, and a pyrimido-indole derivative. In some embodiments, the exogenous factors comprise SCF, IL-7, and an aryl hydrocarbon receptor antagonist. In some embodiments, the exogenous factors comprise SCF, IL-12, and IL-15. In some embodiments, the exogenous factors comprise SCF, IL-12, and FLT3L. In some embodiments, the exogenous factors comprise SCF, IL-12, and a pyrimido-indole derivative. In some embodiments, the exogenous factors comprise SCF, IL-12, and an aryl hydrocarbon receptor antagonist. In some embodiments, the exogenous factors comprise SCF, IL-15, and FLT3L. In some embodiments, the exogenous factors comprise SCF, IL-15, and a pyrimido-indole derivative. In some embodiments, the exogenous factors comprise SCF, IL-15, and an aryl hydrocarbon receptor antagonist. In some embodiments, the exogenous factors comprise SCF, FLT3L, and a pyrimido-indole derivative. In some embodiments, the exogenous factors comprise SCF, FLT3L, and an aryl hydrocarbon receptor antagonist. In some embodiments, the exogenous factors comprise SCF, a pyrimido-indole derivative, and an aryl hydrocarbon receptor antagonist. In some embodiments, the exogenous factors comprise IL-7, IL-12, and IL-15. In some embodiments, the exogenous factors comprise IL-7, IL-12, and FLT3L. In some embodiments, the exogenous factors comprise IL-7, IL-12, and a pyrimido-indole derivative. In some embodiments, the exogenous factors comprise IL-7, IL-12, and a pyrimido-indole derivative. In some embodiments, the exogenous factors comprise IL-7, IL-15, and FLT3L. In some embodiments, the exogenous factors comprise IL-7, IL-15, and a pyrimido-indole derivative. In some embodiments, the exogenous factors comprise IL-7, IL-15, and an aryl hydrocarbon receptor antagonist. In some embodiments, the exogenous factors comprise IL-7, FLT3L, and a pyrimido-indole derivative. In some embodiments, the exogenous factors comprise IL-7, FLT3L, and an aryl hydrocarbon receptor antagonist. In some embodiments, the exogenous factors comprise IL-7, a pyrimido-indole derivative, and an aryl hydrocarbon receptor antagonist. In some embodiments, the exogenous factors comprise IL-12, IL-15, and FLT3L. In some embodiments, the exogenous factors comprise IL-12, IL-15, and a pyrimido-indole derivative. In some embodiments, the exogenous factors comprise IL-12, IL-15, and an aryl hydrocarbon receptor antagonist. In some embodiments, the exogenous factors comprise IL-12, FLT3L, and a pyrimido-indole derivative. In some embodiments, the exogenous factors comprise IL-12, FLT3L, and an aryl hydrocarbon receptor antagonist.

In some embodiments, the exogenous factors comprise SCF, IL-7, IL-12, and IL-15. In some embodiments, the exogenous factors comprise SCF, IL-7, IL-12, and FLT3L. In some embodiments, the exogenous factors comprise SCF, IL-7, IL-12, and a pyrimido-indole derivative. In some embodiments, the exogenous factors comprise SCF, IL-7, IL-12, and an aryl hydrocarbon receptor antagonist. In some embodiments, the exogenous factors comprise SCF, IL-7, IL-15, and FLT3L. In some embodiments, the exogenous factors comprise SCF, IL-7, IL-15, and a pyrimido-indole derivative. In some embodiments, the exogenous factors comprise SCF, IL-7, IL-15, and an aryl hydrocarbon receptor antagonist. In some embodiments, the exogenous factors comprise SCF, IL-7, FLT3L, and a pyrimido-indole derivative. In some embodiments, the exogenous factors comprise SCF, IL-7, FLT3L, and an aryl hydrocarbon receptor antagonist. In some embodiments, the exogenous factors comprise SCF, IL-7, a pyrimido-indole derivative, and an aryl hydrocarbon receptor antagonist. In some embodiments, the exogenous factors comprise SCF, IL-12, IL-15, and FLT3L. In some embodiments, the exogenous factors comprise SCF, IL-12, IL-15, and a pyrimido-indole derivative. In some embodiments, the exogenous factors comprise SCF, IL-12, IL-15, and an aryl hydrocarbon receptor antagonist. In some embodiments, the exogenous factors comprise SCF, IL-12, FLT3L, and a pyrimido-indole derivative. In some embodiments, the exogenous factors comprise SCF, IL-12, FLT3L, and an aryl hydrocarbon receptor antagonist. In some embodiments, the exogenous factors comprise SCF, IL-12, a pyrimido-indole derivative, and an aryl hydrocarbon receptor antagonist. In some embodiments, the exogenous factors comprise SCF, IL-15, FLT3L, and a pyrimido-indole derivative. In some embodiments, the exogenous factors comprise SCF, IL-15, FLT3L, and an aryl hydrocarbon receptor antagonist. In some embodiments, the exogenous factors comprise SCF, IL-15, FLT3L, and an aryl hydrocarbon receptor antagonist. In some embodiments, the exogenous factors comprise SCF, FLT3L, a pyrimido-indole derivative, and an aryl hydrocarbon receptor antagonist. In some embodiments, the exogenous factors comprise IL-7, IL-12, IL-15, and FLT3L. In some embodiments, the exogenous factors comprise IL-7, IL-12, IL-15, and a pyrimido-indole derivative. In some embodiments, the exogenous factors comprise IL-7, IL-12, IL-15, and an aryl hydrocarbon receptor antagonist. In some embodiments, the exogenous factors comprise IL-7, IL-12, FLT3L, and a pyrimido-indole derivative. In some embodiments, the exogenous factors comprise IL-7, IL-12, FLT3L, and an aryl hydrocarbon receptor antagonist. In some embodiments, the exogenous factors comprise IL-7, IL-12, a pyrimido-indole derivative, and an aryl hydrocarbon receptor antagonist. In some embodiments, the exogenous factors comprise IL-7, IL-15, FLT3L, and a pyrimido-indole derivative. In some embodiments, the exogenous factors comprise IL-7, IL-15, FLT3L, and an aryl hydrocarbon receptor antagonist. In some embodiments, the exogenous factors comprise IL-7, IL-15, a pyrimido-indole derivative, and an aryl hydrocarbon receptor antagonist. In some embodiments, the exogenous factors comprise IL-7, FLT3L, a pyrimido-indole derivative, and an aryl hydrocarbon receptor antagonist.

In some embodiments, the exogenous factors comprise SCF, IL-7, IL-12, IL-15, and FLT3L. In some embodiments, the exogenous factors comprise SCF, IL-7, IL-12, IL-15, and a pyrimido-indole derivative. In some embodiments, the exogenous factors comprise SCF, IL-7, IL-12, IL-15, and an aryl hydrocarbon receptor antagonist. In some embodiments, the exogenous factors comprise SCF, IL-7, IL-12, FLT3L, and a pyrimido-indole derivative. In some embodiments, the exogenous factors comprise SCF, IL-7, IL-12, FLT3L, and an aryl hydrocarbon receptor antagonist. In some embodiments, the exogenous factors comprise SCF, IL-7, IL-12, a pyrimido-indole derivative, and an aryl hydrocarbon receptor antagonist. In some embodiments, the exogenous factors comprise SCF, IL-7, IL-15, FLT3L, and a pyrimido-indole derivative. In some embodiments, the exogenous factors comprise SCF, IL-7, IL-15, FLT3L, and an aryl hydrocarbon receptor antagonist. In some embodiments, the exogenous factors comprise SCF, IL-7, IL-15, a pyrimido-indole derivative, and an aryl hydrocarbon receptor antagonist. In some embodiments, the exogenous factors comprise SCF, IL-7, FLT3L, a pyrimido-indole derivative, and an aryl hydrocarbon receptor antagonist. In some embodiments, the exogenous factors comprise SCF, IL-12, IL-15, FLT3L, and a pyrimido-indole derivative. In some embodiments, the exogenous factors comprise SCF, IL-12, IL-15, FLT3L, and an aryl hydrocarbon receptor antagonist. In some embodiments, the exogenous factors comprise SCF, IL-12, IL-15, a pyrimido-indole derivative, and an aryl hydrocarbon receptor antagonist. In some embodiments, the exogenous factors comprise SCF, IL-12, FLT3L, a pyrimido-indole derivative, and an aryl hydrocarbon receptor antagonist. In some embodiments, the exogenous factors comprise SCF, IL-15, FLT3L, a pyrimido-indole derivative, and an aryl hydrocarbon receptor antagonist. In some embodiments, the exogenous factors comprise IL-7, IL-12, IL-15, FLT3L, and a pyrimido-indole derivative. In some embodiments, the exogenous factors comprise IL-7, IL-12, IL-15, FLT3L, and an aryl hydrocarbon receptor antagonist. In some embodiments, the exogenous factors comprise IL-7, IL-12, IL-15, a pyrimido-indole derivative, and an aryl hydrocarbon receptor antagonist. In some embodiments, the exogenous factors comprise IL-7, IL-12, FLT3L, a pyrimido-indole derivative, and an aryl hydrocarbon receptor antagonist. In some embodiments, the exogenous factors comprise IL-7, IL-12, IL-15, FLT3L, and a pyrimido-indole derivative. In some embodiments, the exogenous factors comprise IL-7, IL-12, IL-15, FLT3L, and an aryl hydrocarbon receptor antagonist. In some embodiments, the exogenous factors comprise IL-7, IL-12, IL-15, a pyrimido-indole derivative, and an aryl hydrocarbon receptor antagonist. In some embodiments, the exogenous factors comprise IL-7, IL-12, FLT3L, a pyrimido-indole derivative, and an aryl hydrocarbon receptor antagonist. In some embodiments, the exogenous factors comprise IL-7, IL-15, FLT3L, a pyrimido-indole derivative, and an aryl hydrocarbon receptor antagonist. In some embodiments, the exogenous factors comprise IL-12, IL-15, FLT3L, a pyrimido-indole derivative, and an aryl hydrocarbon receptor antagonist.

In some embodiments, the exogenous factors comprise SCF, IL-7, IL-12, IL-15, FLT3L, and a pyrimido-indole derivative. In some embodiments, the exogenous factors comprise SCF, IL-7, IL-12, IL-15, FLT3L, and an aryl hydrocarbon receptor antagonist. In some embodiments, the exogenous factors comprise SCF, IL-7, IL-12, IL-15, a pyrimido-indole derivative, and an aryl hydrocarbon receptor antagonist. In some embodiments, the exogenous factors comprise SCF, IL-7, IL-12, FLT3L, a pyrimido-indole derivative, and an aryl hydrocarbon receptor antagonist. In some embodiments, the exogenous factors comprise SCF, IL-7, IL-15, FLT3L, a pyrimido-indole derivative, and an aryl hydrocarbon receptor antagonist. In some embodiments, the exogenous factors comprise SCF, IL-12, IL-15, FLT3L, a pyrimido-indole derivative, and an aryl hydrocarbon receptor antagonist. In some embodiments, the exogenous factors comprise IL-7, IL-12, IL-15, FLT3L, a pyrimido-indole derivative, and an aryl hydrocarbon receptor antagonist.

In some embodiments, the exogenous factors comprise SCF, IL-7, and IL-12. In some embodiments, the exogenous factors comprise SCF, IL-7, and IL-15. In some embodiments, the exogenous factors comprise SCF, IL-7, and FLT3L. In some embodiments, the exogenous factors comprise SCF, IL-7, and a pyrimido-indole derivative. In some embodiments, the exogenous factors comprise SCF, IL-7, and an aryl hydrocarbon receptor antagonist. In some embodiments, the exogenous factors comprise SCF, IL-12, and IL-15. In some embodiments, the exogenous factors comprise SCF, IL-12, and FLT3L. In some embodiments, the exogenous factors comprise SCF, IL-12, and a pyrimido-indole derivative. In some embodiments, the exogenous factors comprise SCF, IL-12, and SR1. In some embodiments, the exogenous factors comprise SCF, IL-15, and FLT3L. In some embodiments, the exogenous factors comprise SCF, IL-15, and a pyrimido-indole derivative. In some embodiments, the exogenous factors comprise SCF, IL-15, and SR1. In some embodiments, the exogenous factors comprise SCF, FLT3L, and a pyrimido-indole derivative. In some embodiments, the exogenous factors comprise SCF, FLT3L, and SR1. In some embodiments, the exogenous factors comprise SCF, a pyrimido-indole derivative, and SR1. In some embodiments, the exogenous factors comprise IL-7, IL-12, and IL-15. In some embodiments, the exogenous factors comprise IL-7, IL-12, and FLT3L. In some embodiments, the exogenous factors comprise IL-7, IL-12, and a pyrimido-indole derivative. In some embodiments, the exogenous factors comprise IL-7, IL-12, and a pyrimido-indole derivative. In some embodiments, the exogenous factors comprise IL-7, IL-15, and FLT3L. In some embodiments, the exogenous factors comprise IL-7, IL-15, and a pyrimido-indole derivative. In some embodiments, the exogenous factors comprise IL-7, IL-15, and SR1. In some embodiments, the exogenous factors comprise IL-7, FLT3L, and a pyrimido-indole derivative. In some embodiments, the exogenous factors comprise IL-7, FLT3L, and SR1. In some embodiments, the exogenous factors comprise IL-7, UM729, and SR1. In some embodiments, the exogenous factors comprise IL-12, IL-15, and FLT3L. In some embodiments, the exogenous factors comprise IL-12, IL-15, and UM729. In some embodiments, the exogenous factors comprise IL-12, IL-15, and SR1. In some embodiments, the exogenous factors comprise IL-12, FLT3L, and UM729. In some embodiments, the exogenous factors comprise IL-12, FLT3L, and SR1.

In some embodiments, the exogenous factors comprise SCF, IL-7, IL-12, and IL-15. In some embodiments, the exogenous factors comprise SCF, IL-7, IL-12, and FLT3L. In some embodiments, the exogenous factors comprise SCF, IL-7, IL-12, and UM729. In some embodiments, the exogenous factors comprise SCF, IL-7, IL-12, and SR1. In some embodiments, the exogenous factors comprise SCF, IL-7, IL-15, and FLT3L. In some embodiments, the exogenous factors comprise SCF, IL-7, IL-15, and UM729. In some embodiments, the exogenous factors comprise SCF, IL-7, IL-15, and SR1. In some embodiments, the exogenous factors comprise SCF, IL-7, FLT3L, and UM729. In some embodiments, the exogenous factors comprise SCF, IL-7, FLT3L, and SR1. In some embodiments, the exogenous factors comprise SCF, IL-7, UM729, and SR1. In some embodiments, the exogenous factors comprise SCF, IL-12, IL-15, and FLT3L. In some embodiments, the exogenous factors comprise SCF, IL-12, IL-15, and UM729. In some embodiments, the exogenous factors comprise SCF, IL-12, IL-15, and SR1. In some embodiments, the exogenous factors comprise SCF, IL-12, FLT3L, and UM729. In some embodiments, the exogenous factors comprise SCF, IL-12, FLT3L, and SR1. In some embodiments, the exogenous factors comprise SCF, IL-12, UM729, and SR1. In some embodiments, the exogenous factors comprise SCF, IL-15, FLT3L, and UM729. In some embodiments, the exogenous factors comprise SCF, IL-15, FLT3L, and SR1. In some embodiments, the exogenous factors comprise SCF, IL-15, FLT3L, and SR1. In some embodiments, the exogenous factors comprise SCF, FLT3L, UM729, and SR1. In some embodiments, the exogenous factors comprise IL-7, IL-12, IL-15, and FLT3L. In some embodiments, the exogenous factors comprise IL-7, IL-12, IL-15, and UM729. In some embodiments, the exogenous factors comprise IL-7, IL-12, IL-15, and SR1. In some embodiments, the exogenous factors comprise IL-7, IL-12, FLT3L, and UM729. In some embodiments, the exogenous factors comprise IL-7, IL-12, FLT3L, and SR1. In some embodiments, the exogenous factors comprise IL-7, IL-12, UM729, and SR1. In some embodiments, the exogenous factors comprise IL-7, IL-15, FLT3L, and UM729. In some embodiments, the exogenous factors comprise IL-7, IL-15, FLT3L, and SR1. In some embodiments, the exogenous factors comprise IL-7, IL-15, UM729, and SR1. In some embodiments, the exogenous factors comprise IL-7, FLT3L, UM729, and SR1.

In some embodiments, the exogenous factors comprise SCF, IL-7, IL-12, IL-15, and FLT3L. In some embodiments, the exogenous factors comprise SCF, IL-7, IL-12, IL-15, and UM729. In some embodiments, the exogenous factors comprise SCF, IL-7, IL-12, IL-15, and SR1. In some embodiments, the exogenous factors comprise SCF, IL-7, IL-12, FLT3L, and UM729. In some embodiments, the exogenous factors comprise SCF, IL-7, IL-12, FLT3L, and SR1. In some embodiments, the exogenous factors comprise SCF, IL-7, IL-12, UM729, and SR1. In some embodiments, the exogenous factors comprise SCF, IL-7, IL-15, FLT3L, and UM729. In some embodiments, the exogenous factors comprise SCF, IL-7, IL-15, FLT3L, and SR1. In some embodiments, the exogenous factors comprise SCF, IL-7, IL-15, UM729, and SR1. In some embodiments, the exogenous factors comprise SCF, IL-7, FLT3L, UM729, and SR1. In some embodiments, the exogenous factors comprise SCF, IL-12, IL-15, FLT3L, and UM729. In some embodiments, the exogenous factors comprise SCF, IL-12, IL-15, FLT3L, and SR1. In some embodiments, the exogenous factors comprise SCF, IL-12, IL-15, UM729, and SR1. In some embodiments, the exogenous factors comprise SCF, IL-12, FLT3L, UM729, and SR1. In some embodiments, the exogenous factors comprise SCF, IL-15, FLT3L, UM729, and SR1. In some embodiments, the exogenous factors comprise IL-7, IL-12, IL-15, FLT3L, and UM729. In some embodiments, the exogenous factors comprise IL-7, IL-12, IL-15, FLT3L, and SR1. In some embodiments, the exogenous factors comprise IL-7, IL-12, IL-15, UM729, and SR1. In some embodiments, the exogenous factors comprise IL-7, IL-12, FLT3L, UM729, and SR1. In some embodiments, the exogenous factors comprise IL-7, IL-12, IL-15, FLT3L, and UM729. In some embodiments, the exogenous factors comprise IL-7, IL-12, IL-15, FLT3L, and SR1. In some embodiments, the exogenous factors comprise IL-7, IL-12, IL-15, UM729, and SR1. In some embodiments, the exogenous factors comprise IL-7, IL-12, FLT3L, UM729, and SR1. In some embodiments, the exogenous factors comprise IL-7, IL-15, FLT3L, UM729, and SR1. In some embodiments, the exogenous factors comprise IL-12, IL-15, FLT3L, UM729, and SR1.

In some embodiments, the exogenous factors comprise SCF, IL-7, IL-12, IL-15, FLT3L, and UM729. In some embodiments, the exogenous factors comprise SCF, IL-7, IL-12, IL-15, FLT3L, and SR1. In some embodiments, the exogenous factors comprise SCF, IL-7, IL-12, IL-15, UM729, and SR1. In some embodiments, the exogenous factors comprise SCF, IL-7, IL-12, FLT3L, UM729, and SR1. In some embodiments, the exogenous factors comprise SCF, IL-7, IL-15, FLT3L, UM729, and SR1. In some embodiments, the exogenous factors comprise SCF, IL-12, IL-15, FLT3L, UM729, and SR1. In some embodiments, the exogenous factors comprise IL-7, IL-12, IL-15, FLT3L, UM729, and SR1.

In some embodiments, SCF is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, SCF is present in differentiation media at about 1-50 ng/ml.

In some embodiments, IL-7 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-7 is present in differentiation media at about 1-50 ng/ml.

In some embodiments, IL-12 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-12 is present in differentiation media at about 1-100 ng/ml.

In some embodiments, IL-15 is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-15 is present in differentiation media at about 1-100 ng/ml.

In some embodiments, FLT3L is present in the differentiation media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, FLT3L is present in differentiation media at about 1-100 ng/ml.

In some embodiments, the pyrimido-indole derivative is present in the differentiation media at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 IM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the pyrimido-indole derivative is present in differentiation media at about 0.1-10 μM.

In some embodiments, the pyrimido-indole derivative is UM729. In some embodiments, UM729 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 μM, or any range derivable therein. In some embodiments, UM729 is present in differentiation media at about 0.1-10 μM.

In some embodiments, the aryl hydrocarbon receptor antagonist is present in the differentiation media at a concentration of about 0.1-500 μM, about 1-250 μM, about 1-150 μM, about 5-100 μM, about or about 0.1 μM, about 1 μM, about 2 μM, about 3 μM, about 4 M, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, or any range derivable therein. In some embodiments, the aryl hydrocarbon receptor antagonist is present in differentiation media at about 0.1-10 μM.

In some embodiments, the aryl hydrocarbon receptor antagonist is StemRegenin 1 (SR1). In some embodiments, SR1 is present at a concentration of about 0.1-500 μM, about 1-250 μM, about 1-150 μM, about 5-100 μM, about or about 0.1 μM, about 1 μM, about 2 μM, about 3 μM, about 4 μM, about 5 μM, about 6 μM, about 7 μM, about 8 μM, about 9 M, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about or 100 μM, about or 100 PM, or any range derivable therein. In some embodiments, StemRegenin 1 (SR1) is present in differentiation media at about 0.1-10 μM.

In some embodiments, the NK cell differentiation media comprises SCF, IL-7, IL-12, IL-15, and FLT3L. In some embodiments, the NK cell differentiation media comprises SCF, IL-7, IL-12, IL-15, FLT3L, and a pyrimido-indole derivative. In some embodiments, the NK cell differentiation media comprises SCF, IL-7, IL-12, IL-15, FLT3L, and an aryl hydrocarbon receptor antagonist. In some embodiments, the NK cell differentiation media comprises SCF, IL-7, IL-12, IL-15, FLT3L, a pyrimido-indole derivative, and an aryl hydrocarbon receptor antagonist.

In some embodiments, the NK cell differentiation media comprises SCF, IL-7, IL-12, IL-15, FLT3L, and SR1. In some embodiments, the NK cell differentiation media comprises SCF, IL-7, IL-12, IL-15, FLT3L, and UM729. In some embodiments, the NK cell differentiation media comprises SCF, IL-7, IL-12, IL-15, FLT3L, SR1, and UM729.

In some embodiments, the NK cell differentiation media comprises 1-50 ng/ml SCF, 1-50 ng/ml IL-7, 1-100 ng/ml IL-12, 1-100 ng/ml IL-15, and 1-100 ng/ml FLT3L. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and 0.1-10 μM of a pyrimido-indole derivative. In some embodiments, the NK cell differentiation media comprises 1-50 ng/ml SCF, 1-50 NG/ML IL-7, 1-100 ng/ml IL-12, 1-100 ng/ml IL-15, 1-100 ng/ml FLT3L, and 0.1-10 μM of an aryl hydrocarbon receptor antagonist. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, 0.1-10 μM of a pyrimido-indole derivative, and 0.1-10 μM of an aryl hydrocarbon receptor antagonist.

In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and SR1. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, and 0.1-10 μM UM729. In some embodiments, the NK cell differentiation media comprises 1-50 ng/ml SCF, 1-50 ng/ml IL-7, 1-100 ng/ml IL-12, 1-100 ng/ml IL-15, 1-100 ng/ml FLT3L, 1-10 μM SR1, and 0.1-10 μM UM729.

In some embodiments, the NK cell differentiation media comprises a defined xenogenic-free base media, 1-50 ng/ml SCF, 1-50 ng/ml IL-7, 1-100 ng/ml IL-12, 1-100 ng/ml IL-15, 1-100 ng/ml FLT3L, and 0.1-10 μM SR1. In some embodiments, the NK cell differentiation media comprises a defined xenogenic-freebase media, 1-50 ng/ml SCF, 1-50 ng/ml IL-7, 1-100 ng/ml IL-12, 1-100 ng/ml IL-15, 1-100 ng/ml FLT3L, and 0.1-10 PM UM729. In some embodiments, the NK cell differentiation media comprises a defined xenogenic-freebase media, 1-50 ng/ml SCF, 1-50 ng/ml IL-7, 1-100 ng/ml IL-12, 1-100 ng/ml IL-15, 1-100 ng/ml FLT3L, a 0.1-10 μM SR1, and a 0.1-10 μM UM729.

### NK Cell Expansion Media

In some embodiments, the disclosure provides an expansion media for generating mature NK cells from differentiated NK cells. In some embodiments, differentiated NK cells are generated from HP cells. In some embodiments, the differentiated NK cells produced by the compositions and methods of the disclosure are further expanded to mature NK cells.

In some embodiments, a population of differentiated NK cells are cultured with at least one exogenous factor to form mature NK cells. In some embodiments, the exogenous factor is IL-2. In some embodiments, the exogenous factor is exogenous factor is IL-7. In some embodiments, the exogenous factor is IL-12. In some embodiments, the exogenous factor is IL-15. In some embodiments, the exogenous factor is IL-18. In some embodiments, the exogenous factor is LDL. In some embodiments, the exogenous factor is activation beads. In some embodiments, the exogenous factors are selected from IL-2, IL-7, IL-12, IL-15, IL-18, and activation beads.

In some embodiments, the exogenous factors comprise IL-2 and IL-7. In some embodiments, the exogenous factors comprise IL-2 and IL-12. In some embodiments, the exogenous factors comprise IL-2 and IL-15. In some embodiments, the exogenous factors comprise IL-2 and IL-18. In some embodiments, the exogenous factors comprise IL-2 and activation beads. In some embodiments, the exogenous factors comprise IL-7 and IL-12. In some embodiments, the exogenous factors comprise IL-7 and IL-15. In some embodiments, the exogenous factors comprise IL-7 and IL-18. In some embodiments, the exogenous factors comprise IL-7 and activation beads. In some embodiments, the exogenous factors comprise IL-12 and IL-15. In some embodiments, the exogenous factors comprise IL-12 and IL-18. In some embodiments, the exogenous factors comprise IL-12 and activation beads. In some embodiments, the exogenous factors comprise IL-15 and IL-18. In some embodiments, the exogenous factors comprise IL-15 and activation beads. In some embodiments, the exogenous factors comprise IL-18 and activation beads.

In some embodiments, the exogenous factors comprise IL-2, IL-7, and IL-12. In some embodiments, the exogenous factors comprise IL-2, IL-7, and IL-15. In some embodiments, the exogenous factors comprise IL-2, IL-7, and TL-18. In some embodiments, the exogenous factors comprise IL-2, IL-7, and activation beads. some embodiments, the exogenous factors comprise IL-2, IL-12, and IL-15. In some embodiments, the exogenous factors comprise IL-2, IL-12, and IL-18. In some embodiments, the exogenous factors comprise IL-2, IL-12, and activation beads. In some embodiments, the exogenous factors comprise IL-2, IL-15, and IL-18. In some embodiments, the exogenous factors comprise IL-2, IL-15, and activation beads. In some embodiments, the exogenous factors comprise IL-2, IL-18, and activation beads. In some embodiments, the exogenous factors comprise IL-7, IL-12, and IL-15. In some embodiments, the exogenous factors comprise IL-7, IL-12, and IL-18. In some embodiments, the exogenous factors comprise IL-7, IL-12, and activation beads. In some embodiments, the exogenous factors comprise IL-7, IL-15, and IL-18. In some embodiments, the exogenous factors comprise IL-7, IL-15, and activation beads. In some embodiments, the exogenous factors comprise IL-7, IL-18, and activation beads. In some embodiments, the exogenous factors comprise IL-12, IL-15, and IL-18. In some embodiments, the exogenous factors comprise IL-12, IL-15, and activation beads. In some embodiments, the exogenous factors comprise IL-12, IL-18, and activation beads. In some embodiments, the exogenous factors comprise IL-15, IL-18, and activation beads.

In some embodiments, the exogenous factors comprise IL-2, IL-7, IL-12, and IL-15. In some embodiments, the exogenous factors comprise IL-2, IL-7, IL-12, and IL-18. In some embodiments, the exogenous factors comprise IL-2, IL-7, IL-12, and activation beads. In some embodiments, the exogenous factors comprise IL-2, IL-7, IL-15, and IL-18. In some embodiments, the exogenous factors comprise IL-2, IL-7, IL-15, and activation beads. In some embodiments, the exogenous factors comprise IL-2, IL-7, IL-18, and activation beads. In some embodiments, the exogenous factors comprise IL-2, IL-12, IL-15, and IL-18. In some embodiments, the exogenous factors comprise IL-2, IL-12, IL-15, and activation beads. In some embodiments, the exogenous factors comprise IL-2, IL-12, IL-18, and activation beads. In some embodiments, the exogenous factors comprise IL-2, IL-15, IL-18, and activation beads. In some embodiments, the exogenous factors comprise IL-7, IL-12, IL-15, and IL-18. In some embodiments, the exogenous factors comprise IL-7, IL-12, IL-15, and activation beads. In some embodiments, the exogenous factors comprise IL-7, IL-12, IL-18, and activation beads. In some embodiments, the exogenous factors comprise IL-7, IL-15, IL-18, and activation beads. In some embodiments, the exogenous factors comprise IL-12, IL-15, IL-18, and activation beads.

In some embodiments, the exogenous factors comprise IL-2, IL-7, IL-12, IL-15, and IL-18. In some embodiments, the exogenous factors comprise IL-2, IL-7, IL-12, IL-15, and activation beads. In some embodiments, the exogenous factors comprise IL-2, IL-7, IL-12, IL-18, and activation beads. In some embodiments, the exogenous factors comprise IL-2, IL-7, IL-15, IL-18, and activation beads. In some embodiments, the exogenous factors comprise IL-2, IL-12, IL-15, IL-18, and activation beads. In some embodiments, the exogenous factors comprise IL-7, IL-12, IL-15, IL-18, and activation beads.

In some embodiments, the exogenous factors comprise IL-2, IL-7, IL-12, IL-15, IL-18, and activation beads.

In some embodiments, the exogenous factors comprise IL-2 and IL-7. In some embodiments, the exogenous factors comprise IL-2 and IL-12. In some embodiments, the exogenous factors comprise IL-2 and IL-15. In some embodiments, the exogenous factors comprise IL-2 and IL-18. In some embodiments, the exogenous factors comprise IL-2 and activation beads coated with anti-CD2/anti-NKp46. In some embodiments, the exogenous factors comprise IL-7 and IL-12. In some embodiments, the exogenous factors comprise IL-7 and IL-15. In some embodiments, the exogenous factors comprise IL-7 and IL-18. In some embodiments, the exogenous factors comprise IL-7 and activation beads coated with anti-CD2/anti-NKp46. In some embodiments, the exogenous factors comprise IL-12 and IL-15. In some embodiments, the exogenous factors comprise IL-12 and IL-18. In some embodiments, the exogenous factors comprise IL-12 and activation beads coated with anti-CD2/anti-NKp46. In some embodiments, the exogenous factors comprise IL-15 and IL-18. In some embodiments, the exogenous factors comprise IL-15 and activation beads coated with anti-CD2/anti-NKp46. In some embodiments, the exogenous factors comprise IL-18 and activation beads coated with anti-CD2/anti-NKp46.

In some embodiments, the exogenous factors comprise IL-2, IL-7, and IL-12. In some embodiments, the exogenous factors comprise IL-2, IL-7, and IL-15. In some embodiments, the exogenous factors comprise IL-2, IL-7, and IL-18. In some embodiments, the exogenous factors comprise IL-2, IL-7, and activation beads coated with anti-CD2/anti-NKp46. some embodiments, the exogenous factors comprise IL-2, IL-12, and IL-15. In some embodiments, the exogenous factors comprise IL-2, IL-12, and IL-18. In some embodiments, the exogenous factors comprise IL-2, IL-12, and activation beads coated with anti-CD2/anti-NKp46. In some embodiments, the exogenous factors comprise IL-2, IL-15, and IL-18. In some embodiments, the exogenous factors comprise IL-2, IL-15, and activation beads coated with anti-CD2/anti-NKp46. In some embodiments, the exogenous factors comprise IL-2, IL-18, and activation beads coated with anti-CD2/anti-NKp46. In some embodiments, the exogenous factors comprise IL-7, IL-12, and IL-15. In some embodiments, the exogenous factors comprise IL-7, IL-12, and IL-18. In some embodiments, the exogenous factors comprise IL-7, IL-12, and activation beads coated with anti-CD2/anti-NKp46. In some embodiments, the exogenous factors comprise IL-7, IL-15, and IL-18. In some embodiments, the exogenous factors comprise IL-7, IL-15, and activation beads coated with anti-CD2/anti-NKp46. In some embodiments, the exogenous factors comprise IL-7, IL-18, and activation beads coated with anti-CD2/anti-NKp46. In some embodiments, the exogenous factors comprise IL-12, IL-15, and IL-18. In some embodiments, the exogenous factors comprise IL-12, IL-15, and activation beads coated with anti-CD2/anti-NKp46. In some embodiments, the exogenous factors comprise IL-12, IL-18, and activation beads coated with anti-CD2/anti-NKp46. In some embodiments, the exogenous factors comprise IL-15, IL-18, and activation beads coated with anti-CD2/anti-NKp46.

In some embodiments, the exogenous factors comprise IL-2, IL-7, IL-12, and IL-15. In some embodiments, the exogenous factors comprise IL-2, IL-7, IL-12, and IL-18. In some embodiments, the exogenous factors comprise IL-2, IL-7, IL-12, and activation beads coated with anti-CD2/anti-NKp46. In some embodiments, the exogenous factors comprise IL-2, IL-7, IL-15, and IL-18. In some embodiments, the exogenous factors comprise IL-2, IL-7, IL-15, and activation beads coated with anti-CD2/anti-NKp46. In some embodiments, the exogenous factors comprise IL-2, IL-7, IL-18, and activation beads coated with anti-CD2/anti-NKp46. In some embodiments, the exogenous factors comprise IL-2, IL-12, IL-15, and IL-18. In some embodiments, the exogenous factors comprise IL-2, IL-12, IL-15, and activation beads coated with anti-CD2/anti-NKp46. In some embodiments, the exogenous factors comprise IL-2, IL-12, IL-18, and activation beads coated with anti-CD2/anti-NKp46. In some embodiments, the exogenous factors comprise IL-2, IL-15, IL-18, and activation beads coated with anti-CD2/anti-NKp46. In some embodiments, the exogenous factors comprise IL-7, IL-12, IL-15, and IL-18. In some embodiments, the exogenous factors comprise IL-7, IL-12, IL-15, and activation beads coated with anti-CD2/anti-NKp46. In some embodiments, the exogenous factors comprise IL-7, IL-12, IL-18, and activation beads coated with anti-CD2/anti-NKp46. In some embodiments, the exogenous factors comprise IL-7, IL-15, IL-18, and activation beads coated with anti-CD2/anti-NKp46. In some embodiments, the exogenous factors comprise IL-12, IL-15, IL-18, and activation beads coated with anti-CD2/anti-NKp46.

In some embodiments, the exogenous factors comprise IL-2, IL-7, IL-12, IL-15, and IL-18. In some embodiments, the exogenous factors comprise IL-2, IL-7, IL-12, IL-15, and activation beads coated with anti-CD2/anti-NKp46. In some embodiments, the exogenous factors comprise IL-2, IL-7, IL-12, IL-18, and activation beads coated with anti-CD2/anti-NKp46. In some embodiments, the exogenous factors comprise IL-2, IL-7, IL-15, IL-18, and activation beads coated with anti-CD2/anti-NKp46. In some embodiments, the exogenous factors comprise IL-2, IL-12, IL-15, IL-18, and activation beads coated with anti-CD2/anti-NKp46. In some embodiments, the exogenous factors comprise IL-7, IL-12, IL-15, IL-18, and activation beads coated with anti-CD2/anti-NKp46.

In some embodiments, the exogenous factors comprise IL-2, IL-7, IL-12, IL-15, IL-18, and activation beads coated with anti-CD2/anti-NKp46.

In some embodiments, IL-2 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-2 is present in expansion media at about 1-50 ng/ml.

In some embodiments, IL-7 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-7 is present in expansion media at about 1-50 ng/ml.

In some embodiments, IL-12 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-12 is present in expansion media at about 1-100 ng/ml.

In some embodiments, IL-15 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-15 is present in expansion media at about 1-100 ng/ml.

In some embodiments, IL-18 is present in the expansion media at a concentration of about 0.1-500 ng/ml, about 1-250 ng/ml, about 1-150 ng/ml, about 5-100 ng/ml, about or about 0.1 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about or 100 ng/ml, or any range derivable therein. In some embodiments, IL-18 is present in expansion media at about 1-100 ng/ml.

In some embodiments, activation beads are present in the expansion media.

In some embodiments, activation beads coated with anti-CD2/anti-NKp46 are present in the expansion media at a ratio based on the number of differentiated NK cells. For example, one activation bead is present for each NK cell, or a ratio of 1:1 activation bead to NK cell.

In some embodiments, the activation bead:NK cell ratio is at least 1:1, at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 35:1, at least 40:1, at least 45:1, or at least 50:1.

In some embodiments, the activation bead:NK cell ratio is between 1:1 to 2:1, between 2:1 to 3:1, between 3:1 to 4:1, between 4:1 to 5:1, between 5:1 to 6:1, between 6:1 to 7:1, between 7:1 to 8:1, between 8:1 to 9:1, between 9:1 to 10:1, between 10:1 to 15:1, between 15:1 to 20:1, between 20:1 to 25:1, between 25:1 to 30:1, between 30:1 to 35:1, between 35:1 to 40:1, between 40:1 to 45:1, or between 45:1 to 50:1.

In some embodiments, the NK cell:activation bead ratio is at least 1:1, at least 2:1, at least 3:1, at least 4:1, at least 5:1, at least 6:1, at least 7:1, at least 8:1, at least 9:1, at least 10:1, at least 15:1, at least 20:1, at least 25:1, at least 30:1, at least 35:1, at least 40:1, at least 45:1, or at least 50:1.

In some embodiments, the NK cell:activation bead ratio is between 1:1 to 2:1, between 2:1 to 3:1, between 3:1 to 4:1, between 4:1 to 5:1, between 5:1 to 6:1, between 6:1 to 7:1, between 7:1 to 8:1, between 8:1 to 9:1, between 9:1 to 10:1, between 10:1 to 15:1, between 15:1 to 20:1, between 20:1 to 25:1, between 25:1 to 30:1, between 30:1 to 35:1, between 35:1 to 40:1, between 40:1 to 45:1, or between 45:1 to 50:1.

In some embodiments, the NK cell expansion media comprises IL-2, IL-7, IL-12, IL-15, IL-18, and activation beads. In some embodiments, the NK cell expansion media comprises a defined xenogenic-freebase media, comprises IL-2, IL-7, IL-12, IL-15, IL-18, and activation beads.

In some embodiments, the NK cell expansion media comprises IL-2, IL-7, IL-12, IL-15, IL-18, and activation beads coated with anti-CD2/anti-NKp46. In some embodiments, the NK cell expansion media comprises a defined xenogenic-free base media, comprises IL-2, IL-7, IL-12, IL-15, IL-18, and activation beads coated with anti-CD2/anti-NKp46.

In some embodiments, the NK cell expansion media comprises 1-50 ng/ml IL-2, 1-50 ng/ml IL-7, 1-100 ng/ml IL-12, 1-100 ng/ml IL-15, 1-100 ng/ml IL-18, and activation beads at a cell:bead ratio of 1:1. In some embodiments, the NK cell expansion media comprises a defined xenogenic-freebase media, comprises 1-50 ng/ml IL-2, 1-50 ng/ml IL-7, 1-100 ng/ml IL-12, 1-100 ng/ml IL-15, 1-100 ng/ml IL-18, and activation beads at a cell:bead ratio of 1:1.

In some embodiments, the NK cell expansion media comprises 1-50 ng/ml IL-2, 1-50 ng/ml IL-7, 1-100 ng/ml IL-12, 1-100 ng/ml IL-15, 1-100 ng/ml IL-18, and activation beads coated with anti-CD2/anti-NKp46 at a cell:bead ratio of 1:1. In some embodiments, the NK cell expansion media comprises a defined xenogenic-freebase media, comprises 1-50 ng/ml IL-2, 1-50 ng/ml IL-7, 1-100 ng/ml IL-12, 1-100 ng/ml IL-15, 1-100 ng/ml IL-18, and activation beads coated with anti-CD2/anti-NKp46 at a cell:bead ratio of 1:1.

### Differentiation Methods

In some aspects, the disclosure provides methods of generating hematopoietic progenitors from a stem cell. In some aspects, the disclosure provides methods of generating NK cells from a stem cell. In some aspects, the disclosure provides methods of generating NK cells from a hematopoietic progenitor. In some embodiments, a method of generating NK cells comprises differentiating a stem cell to a hematopoietic progenitor, and differentiating the hematopoietic progenitor to an NK cell.

In some aspects, the disclosure provides methods of generating a common lymphoid progenitor (CLP) from a stem cell. In some embodiments, the methods comprise differentiating a stem cell into a hematopoietic progenitor, and differentiating the hematopoietic progenitor into a CLP. CLPs refer to cells that are precursors to lymphoid cells. CLPs are cells capable of hematopoietic transition to hematopoietic cell-types. In some embodiments, CLPs are CD45+CD7+CD5+/lo CD3−CD56−. In some embodiments, CLPs are CD45+CD5+/lo CD7+. In some aspects, the disclosure provides methods of generating NK cells from CLPs. In some embodiments a method of generating NK cells comprises differentiating a stem cell to a hematopoietic progenitor, differentiating the hematopoietic progenitor into a CLP, and differentiating the CLP into an NK cell.

In some aspects, the disclosure provides methods of generating a preNK cell progenitor (preNKP) from a stem cell. In some embodiments, the methods comprise differentiating a stem cell into a hematopoietic progenitor, differentiating the hematopoietic progenitor into a CLP, and differentiating the CLP into a PreNKP. PreNKPs are intermediate cells between CLPs and NKPs. In some embodiments, PreNKPs are Lin−/CD244+/c− Kit_{low}/IL-7Ra+/FLT3−/CD122− In some aspects, the disclosure provides methods of generating NK cells from preNKPs. In some embodiments a method of generating NK cells comprises differentiating a stem cell to a hematopoietic progenitor, differentiating the hematopoietic progenitor into a CLP, differentiating the CLP into a preNKP, and differentiating the preNKP into an NK cell.

In some aspects, the disclosure provides methods of generating a NK cell precursor (NKP) from a stem cell. In some embodiments, the methods comprise differentiating a stem cell into a hematopoietic progenitor, differentiating the hematopoietic progenitor into a CLP, differentiating the CLP into a PreNKP, and differentiating the PreNKP into an NKP. NKPs are the last cell before the final NK lineage commitment. In some embodiments, NKPs are Lin−/NK1.1-DX5−/IL-7Ra+/CD122+/NKG2D+. In some aspects, the disclosure provides methods of generating NK cells from NKPs. In some embodiments a method of generating NK cells comprises differentiating a stem cell to a hematopoietic progenitor, differentiating the hematopoietic progenitor into a CLP, differentiating the CLP into a preNKP, differentiating the preNKP into NKP, and differentiating the NKP into an NK cell.

In some aspects, the disclosure provides methods of generating an immature NK (iNK) cell from a stem cell. In some embodiments, the methods comprise differentiating a stem cell into a hematopoietic progenitor, differentiating the hematopoietic progenitor into a CLP, differentiating the CLP into a PreNKP, differentiating the PreNKP into an NKP, and differentiating the NKP into an iNK cell. In some aspects, the disclosure provides methods of generating NK cells from an iNK cell. In some embodiments a method of generating NK cells comprises differentiating a stem cell to a hematopoietic progenitor, differentiating the hematopoietic progenitor into a CLP, differentiating the CLP into a preNKP, differentiating the preNKP into an NKP, differentiating the NKP into an iNK cell, and differentiating the iNK cell into an NK cell.

In some aspects, the disclosure provides methods of generating a mature NK (mNK) cell from a stem cell. In some aspects, the disclosure provides methods of generating mature NK cells from an immature NK cell. In some embodiments a method of generating NK cells comprises differentiating a stem cell to a hematopoietic progenitor, differentiating the hematopoietic progenitor into a CLP, differentiating the CLP into a preNKP, differentiating the preNKP into an NKP, differentiating the NKP into an iNK cell, and differentiating the iNK cell into an mNK cell.

In some embodiments, the methods provided herein are xenogenic-free. In some embodiments, the methods provided herein are free of animal-derived raw materials.

### Expression Markers

Differentiation of source cells into NK cells can be assessed by detecting markers, e.g., CD56, CD94, CD117, NKG2D, DNAM-1 and NKp46, by, for example, flow cytometry. Differentiation can also be assessed by the morphological characteristics of NK cells, e.g., large size, high protein synthesis activity in the abundant endoplasmic reticulum (ER), and/or preformed granules. Maturation of NK cells can be assessed by detecting one or more functionally relevant makers, for example, CD94, CD161, NKp44, DNAM-1, 2B4, NKp46, CD94, KIR, and the NKG2 family of activating receptors (e.g., NKG2D). Maturation of NK cells can also be assessed by detecting specific markers during different developmental stages. For example, in one embodiment, preNKP cells are CD34+, CD45RA+, CD10+, CD117− and/or CD161−. In another embodiment, immature NK cells are CD34−, CD117+, CD161+, NKp46− and/or CD94/NKG2A−. In another embodiment, CD56bright NK cells are CD117+, NKp46+, CD94/NKG2A+, CD16−, and/or KIR+/−. In another embodiment, CD56dim NK cells are CD117−, NKp46+, CD94/NKG2A+/−, CD16+, and/or KIR+. In a specific embodiment, maturation of NK cells (e.g., TSNK cells) is determined by the percentage of NK cells (e.g., TSNK cells) that are CD161−, CD94+ and/or NKp46+. In a more specific embodiment, at least 10%, 20%, 25%, 30%, 35%, 40%, 50%, 55%, 60%, 65% or 70% of mature NK cells (e.g., TSNK cells) are NKp46+. In another more specific embodiments, at least 10%, 20%, 25%, 30%, 35%, 40%, 45% or 50% of mature NK cells (e.g., TSNK cells) are CD94+. In another more specific embodiments, at least 10%, 20%, 25%, 30%, 35%, 40%, 45% or 50% of mature NK cells (e.g., TSNK cells) are CD161−.

In some embodiments, the differentiation of source cells into NK cells are assessed by detecting the expression level of, e.g., CD3, CD7 or CD127, CD10, CD14, CD15, CD16, CD33, CD34, CD56, CD94, CD117, CD161, NKp44, NKp46, NKG2D, DNAM-1, 2B4 or TO-PRO-3, using, e.g., antibodies to one or more of these cell markers. Such antibodies can be conjugated to a detectable label, for example, as fluorescent label, e.g., FITC, R-PE, PerCP, PerCP-Cy5.5, APC, APC-Cy7 or APC-H7.

### Source Cells

In some embodiments, NK cells are generated from source cells. Any progenitor cell known in the art may be used as a source cell in the methods of the disclosure.

In some embodiments, the source cells are hESCs. In some embodiments, the source cells are iPSCs. An NK cell derived from iPSCs may alternatively be referred to as iPSC-derived NK cells.

In immunotherapy, source cells be allogeneic or autologous, meaning from a donor or from the subject, respectively. In some embodiments, the source cells are allogeneic. In some embodiments, the source cells are autologous.

In some embodiments, source cells are peripheral blood cells. As used herein, the term “peripheral blood cell” is used to refer to cells that originate from circulating blood and comprise hematopoietic stem cells that are capable of proliferation, selectable differentiation, and maturation. As such, peripheral blood NK cells may alternatively be referred to as differentiated blood-derived NK cells (bdNK).

In some embodiments, source cells include hematopoietic stem cells, characterized as being CD34+ and/or CD45+.

In some embodiments, source cells include common lymphoid progenitor cells, characterized as being CD45+CD7+CD56−.

In some embodiments, NK cells may be generated from induced pluripotent stem cells (iPSCs). iPSCs are a type of pluripotent stem cell derived from adult somatic cells that have been genetically reprogrammed to an embryonic stem cell-like state through the forced expression of genes and factors important for maintaining the defining properties of embryonic stem cells. iPSCs may be generated from tissues with somatic cells, including, but not limited to, the skin, dental tissue, peripheral blood, and urine. To generate iPSCs, somatic cells may be reprogrammed through methods including, but not limited to, the transient expression of reprogramming factors, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, CRISPR-based gene editing, or recombinant proteins. Methods for generating iPSCs are disclosed in U.S. Pat. No. 9,315,779 B2, U.S. Ser. No. 10/370,452 B2, U.S. Ser. No. 11/319,555 B2, and US20210015859A1, which are incorporated by reference in their entirety.

### Mesoderm/Embryoid Body Formation

In some embodiments, the methods described herein comprise generating mesoderm cells from iPSCs and/or hESCs. As stem cells begin to differentiate, three distinct germ layers are formed: the ectoderm, mesoderm, and endoderm. Immune cells, such as NK cells, differentiate from mesoderm cells. In some embodiments, the mesoderm cells produced by the methods of the disclosure are further differentiated to NK cells.

The mesoderm formation step may comprise contacting the iPSC or hESC cell population with one or more factors, in a defined expansion media, for specified period of time. In some embodiments, mesoderm cells are formed from embryoid bodies.

In some embodiments, stem cells are contacted with a differentiation media described herein for a period of time to generate mesoderm cells and/or embryoid bodies. In some embodiments, the period of time sufficient to generate mesoderm cells from stem cells is at least 24 hours, at least 48 hours, at least 72 hours, at least 96 hours, or at least 120 hours.

In some embodiments, the mesoderm formation step has duration of between 12 hours to 24 hours, between 24 hours to 48 hours, between 48 hours to 72 hours, between 72 hours to 96 hours, or between 96 hours to 120 hours.

In some embodiments, a cell derived from a source cell is disposed in a vessel to induce the cells to aggregate and form clusters. In some embodiments, the vessel is a plate with wells or microwells, such as a 96-well plate and/or an Aggrewell™ plate (microwell plate; STEMCELL Technologies Inc., Vancouver, Canada). In some embodiments, using, for example, an Aggrewell™ plate, cell clusters are prepared in a plate having microwells to form aggregates of cells of uniform size and shape. In some embodiments, at least 1 cell, at least 10 cells, at least 100 cells, at least 1,000 cells, at least 10,000 cells, or at least 50,000 cells are seeded in each well. In some embodiments, between 1 cell to 10 cells, between 10 cells to 100 cells, between 100 cells to 1,000 cells, between 1,000 cells to 10,000 cells, or between 10,000 cells to 50,000 cells are seeded in each well.

Differentiation into Hematopoietic Progenitors

In some aspects, the disclosure provides methods of generating NK cells from a hematopoietic progenitor cells. In some embodiments, a method of generating NK cells comprises differentiating the hematopoietic progenitor to an NK cell. In some embodiments, the methods provided herein are xenogenic-free.

An aspect of the disclosure is that the method of producing NK cells may include a hematopoietic progenitor differentiation step. The hematopoietic progenitor differentiation step may comprise contacting the embryoid body cell population with one or more factors, in a defined differentiation media, for specified period of time, thereby inducing formation of hematopoietic progenitors in the cell population. The hematopoietic progenitors are then defined by expressing a combination of markers.

In some embodiments, mesoderm and/or embryoid body cells are contacted with a differentiation media described herein for a period of time to generate hematopoietic progenitor cells. In some embodiments, the period of time sufficient to generate hematopoietic progenitor cells from mesoderm and/or embryoid body cells is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days.

In some embodiments, the differentiation into hematopoietic progenitors step has duration of between 1 day to 2 days, between 2 days to 3 days, between 3 days to 4 days, between 4 days to 5 days, between 5 days to 6 days, between 6 days to 7 days, between 7 days to 8 days, between 8 days to 9 days, between 9 days to 10 days, between 10 days to 11 days, between 11 days to 12 days, between 12 days to 13 days, between 13 days to 14 days, between 14 days to 15 days, between 15 days to 16 days, between 16 days to 17 days, between 17 days to 18 days, between 18 days to 19 days, or between 19 days to 20 days.

In some embodiments, the hematopoietic progenitor cells express CD34, CD43 and CD45. In some embodiments, the method of the disclosure increases the percentage of CD34+CD43+CD45+ triple-positive cells.

In some embodiments, the methods of the disclosure generate a population of cells from the iPSCs with a purity of at least 40%, at least 50%, at least 60%, at least 70%, at least 80%, at least 90%/o or 100% of CD34+CD43+CD45+ triple-positive cells.

In some embodiments, the methods of the disclosure generate a population of cells from the iPSCs with a purity of between 40% to 50%, between 50% to 60%, between 60% to 70%, between 70% to 80%, between 80%/o to 90%, or between 90% to 100% of CD34+CD43+CD45+ triple-positive cells.

In some embodiments, the methods and compositions of the disclosure increase the yield ratio of hematopoietic progenitors (HP) from a source cell (SC), such as an hESC or an iPSCs. In some embodiments, the ratio of HPs to source cells (SCs) is shown as HPs/SCs. In some embodiments, the ratio of HPs to hESC is shown as HPs/hESC. In some embodiments, the ratio of HPs to iPSCs is shown as HPs/iPSCs.

In some embodiments, the yield ratio of HPs/SCs is about 1:1, about 2:1, about 3:1, about 4:1, about 5:1, about 6:1, about 7:1, about 8:1, about 9:1, or about 10:1. In some embodiments, the yield ratio of HPs/SCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, or from about 9:1 to about 10:1.

In some embodiments, the methods and compositions of the disclosure increase the yield ratio the yield ratio of HPs/SCs. For example, the methods and compositions increase the HPs/SCs from about 2:1 to about 4:1, or by about 2. In some embodiments, the methods and compositions of the disclosure increase the yield ratio the yield ratio of HPs/SCs by about 1, about 2, about 3, about 4, about 5, about 6, about 7, about 8, about 9, or about 10. In some embodiments, the yield ratio of HPs/SCs is from about 1 to about 2, from about 2 to about 3, from about 3 to about 4, from about 4 to about 5, from about 5 to about 6, from about 6 to about 7, from about 7 to about 8, from about 8 to about 9, or from about 9 to about 10.

In some embodiments, the yield ratio of HPs to stem cells (StCs) (HP/StCs) is about 1:1, about 2:1, about 3:1, about 4:1, about 5:1, about 6:1, about 7:1, about 8:1, about 9:1, or about 10:1. In some embodiments, the yield ratio of HPs/StCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, or from about 9:1 to about 10:1.

In some embodiments, the methods and compositions of the disclosure increase the yield ratio the yield ratio of HPs to stem cells (StCs) (HP/StCs). For example, the methods and compositions increase the HPs/StCs from about 2:1 to about 4:1, or by about 2. In some embodiments, the methods and compositions of the disclosure increase the yield ratio the yield ratio of HPs/StCs by about 1, about 2, about 3, about 4, about 5, about 6, about 7, about 8, about 9, or about 10. In some embodiments, the yield ratio of HPs/StCs is from about 1 to about 2, from about 2 to about 3, from about 3 to about 4, from about 4 to about 5, from about 5 to about 6, from about 6 to about 7, from about 7 to about 8, from about 8 to about 9, or from about 9 to about 10.

In some embodiments, the yield ratio of HPs to iPSCs (HP/iPSCs) is about 1:1, about 2:1, about 3:1, about 4:1, about 5:1, about 6:1, about 7:1, about 8:1, about 9:1, or about 10:1. In some embodiments, the yield ratio of HPs/iPSCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, or from about 9:1 to about 10:1.

In some embodiments, the methods and compositions of the disclosure increase the yield ratio the yield ratio of HPs to iPSCs (HP/iPSCs). For example, the methods and compositions increase the HPs to iPSCs (HP/iPSCs) from about 2:1 to about 4:1, or by about 2. In some embodiments, the methods and compositions of the disclosure increase the yield ratio the yield ratio of HPs to iPSCs (HP/iPSCs) by about 1, about 2, about 3, about 4, about 5, about 6, about 7, about 8, about 9, or about 10. In some embodiments, the yield ratio of HPs to iPSCs (HP/iPSCs) is from about 1 to about 2, from about 2 to about 3, from about 3 to about 4, from about 4 to about 5, from about 5 to about 6, from about 6 to about 7, from about 7 to about 8, from about 8 to about 9, or from about 9 to about 10.

In some embodiments, the yield ratio of HPs to hESCs (HP/hESCs) is about 1:1, about 2:1, about 3:1, about 4:1, about 5:1, about 6:1, about 7:1, about 8:1, about 9:1, or about 10:1. In some embodiments, the yield ratio of HPs/hESCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, or from about 9:1 to about 10:1.

In some embodiments, the methods and compositions of the disclosure increase the yield ratio the yield ratio of HPs to hESCs (HP/hESCs). For example, the methods and compositions increase the HPs to hESCs (HP/hESCs) from about 2:1 to about 4:1, or by about 2. In some embodiments, the methods and compositions of the disclosure increase the yield ratio the yield ratio of HPs to hESCs (HP/hESCs) by about 1, about 2, about 3, about 4, about 5, about 6, about 7, about 8, about 9, or about 10. In some embodiments, the yield ratio of HPs to hESCs (HP/hESCs) is from about 1 to about 2, from about 2 to about 3, from about 3 to about 4, from about 4 to about 5, from about 5 to about 6, from about 6 to about 7, from about 7 to about 8, from about 8 to about 9, or from about 9 to about 10.

Differentiation into NK Cells

In some aspects, the disclosure provides methods of generating NK cells from a differentiated NK cells. In some embodiments, a method of generating NK cells comprises differentiating the NK cells. In some embodiments, the methods provided herein are xenogenic-free.

An aspect of the disclosure is that the method of producing NK cells may include a NK differentiation step. The NK differentiation step may comprise contacting the HP cell population with one or more factors, in a defined differentiation media, for specified period of time, thereby inducing formation of NK cells in the cell population. The NK cells are then defined by expressing a combination of markers.

In some embodiments, hematopoietic progenitor cells are contacted with a differentiation media described herein for a period of time to generate NK cells. In some embodiments, the period of time sufficient to generate NK cells from hematopoietic progenitor cells is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, or at least 40 days,

In some embodiments, the NK differentiation step has duration of between 1 day to 2 days, between 2 days to 3 days, between 3 days to 4 days, between 4 days to 5 days, between 5 days to 6 days, between 6 days to 7 days, between 7 days to 8 days, between 8 days to 9 days, between 9 days to 10 days, between 10 days to 11 days, between 11 days to 12 days, between 12 days to 13 days, between 13 days to 14 days, between 14 days to 15 days, between 15 days to 16 days, between 16 days to 17 days, between 17 days to 18 days, between 18 days to 19 days, or between 19 days to 20 days, between 20 days to 21 days, between 21 days to 22 days, between 22 days to 23 days, between 23 days to 24 days, between 24 days to 25 days, between 25 days to 26 days, between 26 days to 27 days, between 27 days to 28 days, between 28 days to 29 days, between 29 days to 30 days, between 30 days to 31 days, between 31 days to 32 days, between 32 days to 33 days, between 33 days to 34 days, between 34 days to 35 days, between 35 days to 36 days, between 36 days to 37 days, between 37 days to 38 days, between 38 days to 39 days, or between 39 days to 40 days.

In some embodiments, the differentiated NK cells comprise the markers CD34, CD43, CD45, and LFA1. In some embodiments, the method of the disclosure increases the percentage of CD34+CD43+CD45+ LFA1+ quadruple-positive cells.

In some embodiments, the methods of the disclosure generate a population of NK cells from the hematopoietic progenitor cells with a purity of at least 40%, at least 50%, at least 60%, at least 70%, at least 80%, at least 90% or 100% of CD34+CD43+CD45+ LFA1+ quadruple-positive cells.

In some embodiments, the methods of the disclosure generate a population of NK cells from the hematopoietic progenitor cells with a purity of between 40% to 50%, between 50% to 60%, between 60% to 70%, between 70% to 80%, between 80% to 90%, or between 90% to 100% of CD34+CD43+CD45+ LFA1+ quadruple-positive cells.

In some embodiments, the methods and compositions of the disclosure increase the yield ratio of NK cells from a source cell (SC), such as a hESC or an iPSCs. In some embodiments, the ratio of NKs to source cells (SCs) is shown as NKs/SCs. In some embodiments, the ratio of NKs to hESC is shown as NKs/hESC. In some embodiments, the ratio of NKs to iPSCs is shown as NKs/iPSCs.

In some embodiments, the yield ratio of NKs/SCs is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of NKs/SCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of NKs/SCs. For example, the methods and compositions increase the NKs/SCs from about 2:1 to about 4:1, or by about 2. In some embodiments, the methods and compositions of the disclosure increase the yield ratio the yield ratio of NKs/SCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of NKs/SCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of NK cells to stem cells (StCs) (NK/StCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of NK/StCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of NK/StCs. For example, the methods and compositions increase the NK/StCs from about 2:1 to about 4:1, or by about 2. In some embodiments, the methods and compositions of the disclosure increase the yield ratio the yield ratio of NK/StCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of NK/StCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of NK cells to hESCs (NK/hESCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of NK/hESCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of NK/hESCs. For example, the methods and compositions increase the NK/hESCs from about 2:1 to about 4:1, or by about 2. In some embodiments, the methods and compositions of the disclosure increase the yield ratio the yield ratio of NK/hESCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of NK/hESCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of NK cells to iPSCs (NK/iPSCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of NK/iPSCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of NK/iPSCs. For example, the methods and compositions increase the NK/iPSCs from about 2:1 to about 4:1, or by about 2. In some embodiments, the methods and compositions of the disclosure increase the yield ratio the yield ratio of NK/iPSCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of NK/iPSCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

### NK Cell Maturation

In some aspects, the disclosure provides methods of generating mature NK cells from a differentiated NK cells. In some embodiments, a method of generating mature NK cells comprises differentiating the NK cells. In some embodiments, the methods provided herein are xenogenic-free.

An aspect of the disclosure is that the method of producing NK cells may include a NK maturation step. The NK maturation step may comprise contacting the differentiated NK cell population with one or more factors, in a defined expansion media, for specified period of time, thereby inducing NK cell maturation in the cell population. The mature NK cells are then defined by expressing a combination of markers.

In some embodiments, differentiated NK cells are contacted with a maturation media described herein for a period of time to generate mature NK cells. In some embodiments, the period of time sufficient to generate mature NK cells from hematopoietic progenitor cells is at least 24 hours, at least 48 hours, at least 72 hours, at least 96 hours, at least 120 hours at least 144 hours, at least 168 hours, at least 192 hours, at least 216 hours, or at least 240 hours.

In some embodiments, the maturation step has duration of between 12 hours to 24 hours, between 24 hours to 48 hours, between 48 hours to 72 hours, between 72 hours to 96 hours, between 96 hours to 120 hours, between 120 hours to 144 hours, between 144 hours to 168 hours, between 168 hours to 192 hours, between 192 hours to 216 hours, or between 216 hours to 240 hours.

In some embodiments, the mature NK cells comprise the markers CD34, CD43, CD45, and LFA1. In some embodiments, the method of the disclosure increases the percentage of CD34+CD43+CD45+ LFA1+ quadruple-positive cells. In some embodiments, the method increases expression of activation markers. In some embodiments, the activation markers comprise NKp46, NKG2D, LFA1, and/or CD16. In some embodiments, the method decrease expression of inhibitory markers. In some embodiments, the inhibitory markers comprise CD161 and CD73.

In some embodiments, the methods of the disclosure generate a population of mature NK cells from the differentiated NK cells with a purity of at least 40%, at least 50%, at least 60%, at least 70%, at least 80%, at least 90% or 100% of CD34+CD43+CD45+ LFA1+ NKp46+ NKG2D+ LFA1+CD161-CD73− cells.

In some embodiments, the methods of the disclosure generate a population of mature NK cells from the differentiated NK cells with a purity of between 40% to 50%, between 50% to 60%, between 60% to 70%, between 70% to 80%, between 80% to 90%, or between 90% to 100% of CD34+CD43+CD45+ LFA1+ NKp46+ NKG2D+ LFA1+CD161−CD73− cells.

In some embodiments, the maturation step decreases the population of CD56− cells.

In some embodiments, the methods of the disclosure generate a population of mature NK cells from the differentiated NK cells with a purity of at least 40%, at least 50%, at least 60%, at least 70%, at least 80%, at least 90% or 100% of CD56− cells.

In some embodiments, the methods of the disclosure generate a population of mature NK cells from the differentiated NK cells with a purity of between 40% to 50%, between 50% to 60%, between 60% to 70%, between 70% to 80%, between 80% to 90%, or between 90% to 100% of CD56− cells.

In some embodiments, the methods and compositions of the disclosure increase the yield ratio of mature NK cells from a source cell (SC), such as a hESC or an iPSCs. In some embodiments, the ratio of mature NKs to source cells (SCs) is shown as mature NKs/SCs. In some embodiments, the ratio of mature NKs to hESC is shown as mature NKs/hESC. In some embodiments, the ratio of mature NKs to iPSCs is shown as mature NKs/iPSCs.

In some embodiments, the yield ratio of mature NKs/SCs is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of mature NKs/SCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of mature NKs/SCs. For example, the methods and compositions increase the mature NKs/SCs from about 2:1 to about 4:1, or by about 2. In some embodiments, the methods and compositions of the disclosure increase the yield ratio the yield ratio of mature NKs/SCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of mature NKs/SCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of mature NK cells to stem cells (StCs) (mature NK/StCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of mature NK/StCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of mature NK/StCs. For example, the methods and compositions increase the mature NK/StCs from about 2:1 to about 4:1, or by about 2. In some embodiments, the methods and compositions of the disclosure increase the yield ratio the yield ratio of mature NK/StCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of mature NK/StCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of mature NK cells to hESCs (mature NK/hESCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of mature NK/hESCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of mature NK/hESCs. For example, the methods and compositions increase the mature NK/hESCs from about 2:1 to about 4:1, or by about 2. In some embodiments, the methods and compositions of the disclosure increase the yield ratio the yield ratio of mature NK/hESCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of mature NK/hESCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

In some embodiments, the yield ratio of mature NK cells to iPSCs (mature NK/iPSCs) is about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, or about 100:1. In some embodiments, the yield ratio of mature NK/iPSCs is from about 1:1 to about 2:1, from about 2:1 to about 3:1, from about 3:1 to about 4:1, from about 4:1 to about 5:1, from about 5:1 to about 6:1, from about 6:1 to about 7:1, from about 7:1 to about 8:1, from about 8:1 to about 9:1, from about 9:1 to about 10:1, from about 10:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some embodiments, the methods and compositions of the disclosure increase the yield ratio the yield ratio of mature NK/iPSCs. For example, the methods and compositions increase the mature NK/iPSCs from about 2:1 to about 4:1, or by about 2. In some embodiments, the methods and compositions of the disclosure increase the yield ratio the yield ratio of mature NK/iPSCs by about 1, about 2, about 3, about 4, about 5, about 6, about 7, about 8, about 9, about 10, about 15, about 20, about 25, about 30, about 35, about 40, about 45, about 50, about 60, about 70, about 80, about 90, or about 100. In some embodiments, the yield ratio of mature NK/iPSCs is from about 1 to about 2, from about 2 to about 3, from about 3 to about 4, from about 4 to about 5, from about 5 to about 6, from about 6 to about 7, from about 7 to about 8, from about 8 to about 9, from about 9 to about 10, from about 10 to about 15, from about 15 to about 20, from about 20 to about 25, from about 25 to about 30, from about 30 to about 35, from about 35 to about 40, from about 40 to about 45, from about 45 to about 50, from about 50 to about 60, from about 60 to about 70, from about 70 to about 80, from about 80 to about 90, or from about 90 to about 100.

### Exemplary Differentiation Methods

In some embodiments, a method of differentiating stem cells into hematopoietic progenitors comprises contacting a population of stem cells with a xenogenic-free differentiation media comprising BMP4, FGF2, and VEGF. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, and 5-100 ng/mL VEGF. In some embodiments, a method of differentiating stem cells into hematopoietic progenitors comprises contacting a population of stem cells with a xenogenic-free differentiation media comprising BMP4, FGF2, and VEGF, for 12 to 120 hours. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, and 5-100 ng/mL VEGF, for 12 to 120 hours.

In some embodiments, a method of differentiating stem cells into hematopoietic progenitors comprises contacting a population of stem cells with a xenogenic-free differentiation media comprising BMP4, FGF2, VEGF, and a ROCK inhibitor. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, 5-100 ng/mL VEGF, and 0.1-20 μM of a ROCK inhibitor. In some embodiments, a method of differentiating stem cells into hematopoietic progenitors comprises contacting a population of stem cells with a xenogenic-free differentiation media comprising BMP4, FGF2, VEGF, and a ROCK inhibitor, for 12-120 hours. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, 5-100 ng/mL VEGF, and 0.1-20 μM of a ROCK inhibitor, for 12 to 120 hours.

In some embodiments, a method of differentiating stem cells into hematopoietic progenitors comprises contacting a population of stem cells with a serum-free differentiation media comprising BMP4, FGF2, VEGF, and Y27632. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, 5-100 ng/mL VEGF, and 0.1-20 μM Y27632. In some embodiments, a method of differentiating stem cells into hematopoietic progenitors comprises contacting a population of stem cells with a xenogenic-free differentiation media comprising BMP4, FGF2, VEGF, and Y27632, for 12-120 hours. In some embodiments, a method of differentiating stem cells into hematopoietic progenitors comprises contacting a population of stem cells with a xenogenic-free differentiation media comprising 1-50 ng/mL BMP4, 1-50 ng/mL FGF2, 5-100 ng/mL VEGF, and 0.1-20 μM Y27632, for 12 to 120 hours.

In some embodiments, a method of differentiating stem cells into hematopoietic progenitors comprises (i) contacting a population of stem cells with a first xenogenic-free differentiation media comprising BMP4, FGF2, and VEGF to generate a population of mesoderm cells, and (ii) contacting the population of mesoderm cells with a second xenogenic-free differentiation media comprising BMP4, FGF2, VEGF, SCF, LDL, TPO, and a PI3K inhibitor to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, and 5-100 ng/mL VEGF to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor to generate hematopoietic progenitors. In some embodiments, a method of differentiating stem cells into hematopoietic progenitors comprises (i) contacting a population of stem cells with a first xenogenic-free differentiation media comprising BMP4, FGF2, and VEGF for 12-120 hours to generate a population of mesoderm cells, and (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, and 5-100 ng/mL VEGF for 12-120 hours to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor for 2-20 days to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and a ROCK inhibitor to generate a population of mesoderm cells, and (ii) contacting the population of mesoderm cells with a second xenogenic-free differentiation media comprising BMP4, FGF2, VEGF, SCF, LDL, TPO, and a PI3K inhibitor to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM of a ROCK inhibitor to generate a population of mesoderm cells, and (ii) contacting the population of mesoderm cells with a second xenogenic-free differentiation media comprising −50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and a ROCK inhibitor for 12-120 hours to generate a population of mesoderm cells, and (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM of a ROCK inhibitor for 12-120 hours to generate a population of mesoderm cells, and (ii) contacting the population of mesoderm cells with a second xenogenic-free differentiation media comprising 5-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor for 2-20 days to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and Y27632 to generate a population of mesoderm cells, and (ii) contacting the population of mesoderm cells with a second xenogenic-free differentiation media comprising BMP4, FGF2, VEGF, SCF, LDL, TPO, and a PI3K inhibitor to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM Y27632 to generate a population of mesoderm cells, and (ii) contacting the population of mesoderm cells with a second xenogenic-free differentiation media comprising −50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and Y27632 for 12-120 hours to generate a population of mesoderm cells, and (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM Y27632 for 12-120 hours to generate a population of mesoderm cells, and (ii) contacting the population of mesoderm cells with a second xenogenic-free differentiation media comprising −50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM of a PI3K inhibitor for 2-20 days to generate hematopoietic progenitors.

In some embodiments, a method of differentiating stem cells into hematopoietic progenitors comprises (i) contacting a population of stem cells with a first xenogenic-free differentiation media comprising BMP4, FGF2, and VEGF to generate a population of mesoderm cells, and (ii) contacting the population of mesoderm cells with a second xenogenic-free differentiation media comprising BMP4, FGF2, VEGF, SCF, LDL, TPO, and LY294002 to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, and 5-100 ng/mL VEGF to generate a population of mesoderm cells, and (ii) contacting the population of mesoderm cells with a second xenogenic-free differentiation media comprising −50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 to generate hematopoietic progenitors. In some embodiments, a method of differentiating stem cells into hematopoietic progenitors comprises (i) contacting a population of stem cells with a first xenogenic-free differentiation media comprising BMP4, FGF2, and VEGF for 12-120 hours to generate a population of mesoderm cells, and (ii) contacting the population of mesoderm cells with a second xenogenic-free differentiation media comprising BMP4, FGF2, VEGF, SCF, LDL, TPO, and LY294002 for 2-20 days to generate hematopoietic progenitors.

In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, and 5-100 ng/mL VEGF for 12-120 hours to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 for 2-20 days to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and a ROCK inhibitor to generate a population of mesoderm cells, and (ii) contacting the population of mesoderm cells with a second xenogenic-free differentiation media comprising BMP4, FGF2, VEGF, SCF, LDL, TPO, and LY294002 to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM of a ROCK inhibitor to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and a ROCK inhibitor for 12-120 hours to generate a population of mesoderm cells, and (ii) contacting the population of mesoderm cells with a second xenogenic-free differentiation media comprising BMP4, FGF2, VEGF, SCF, LDL, TPO, and LY294002 for 2-20 days to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM of a ROCK inhibitor for 12-120 hours to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 for 2-20 days to generate hematopoietic progenitors.

In some embodiments, a method of differentiating stem cells into hematopoietic progenitors comprises (i) contacting a population of stem cells with a first xenogenic-free differentiation media comprising BMP4, FGF2, VEGF, and Y27632 to generate a population of mesoderm cells, and (ii) contacting the population of mesoderm cells with a second xenogenic-free differentiation media comprising BMP4, FGF2, VEGF, SCF, LDL, TPO, and LY294002 to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM Y27632 to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 to generate hematopoietic progenitors. In some embodiments, a method of differentiating stem cells into hematopoietic progenitors comprises (i) contacting a population of stem cells with a first xenogenic-free differentiation media comprising BMP4, FGF2, VEGF and Y27632 for 12-120 hours to generate a population of mesoderm cells, and (ii) contacting the population of mesoderm cells with a second xenogenic-free differentiation media comprising BMP4, FGF2, VEGF, SCF, LDL, TPO, and LY294002 for 2-20 days to generate hematopoietic progenitors. In some embodiments, a method of differentiating stem cells into hematopoietic progenitors comprises (i) contacting a population of stem cells with a first xenogenic-free differentiation media comprising 1-50 ng/mL BMP4, 1-50 ng/mL FGF2, 5-100 ng/mL VEGF, and 0.1-20 μM Y27632 for 12-120 hours to generate a population of mesoderm cells, and (ii) contacting the population of mesoderm cells with a second xenogenic-free differentiation media comprising 1-50 ng/mL BMP4, 1-50 ng/mL FGF2, 5-100 ng/mL VEGF, about 1-100 ng/mL SCF, about 1-50 ug/mL LDL, about 1-100 ng/mL TPO, and 0.1-100 uM LY294002 for 2-20 days to generate hematopoietic progenitors.

In some embodiments, the hematopoietic progenitor cell formation step is followed by an NK cell differentiation step. In some embodiments, the method of differentiating HPs into NK comprises contacting a population of HP cells with a xenogenic-free differentiation media comprising SCF, IL-7, IL-15, IL-12, FLT3L, a pyrimido-indole derivative, and an aryl hydrocarbon receptor antagonist. In some embodiments, the method of differentiating HPs into NK comprises contacting a population of HP cells with a xenogenic-free differentiation media comprising SCF, IL-7, IL-15, IL-12, FLT3L, a pyrimido-indole derivative, and an aryl hydrocarbon receptor antagonist for 15-25 days. In some embodiments, the method of differentiating HPs into NK comprises contacting a population of HP cells with a xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist. In some embodiments, the method of differentiating HPs into NK comprises contacting a population of HP cells with a xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist, for 15 to 25 days.

In some embodiments, the method of differentiating HPs into NK comprises contacting a population of HP cells with a xenogenic-free differentiation media comprising SCF, IL-7, IL-15, IL-12, FLT3L, UM729, and SR1. In some embodiments, the method of differentiating HPs into NK comprises contacting a population of HP cells with a xenogenic-free differentiation media comprising SCF, IL-7, IL-15, IL-12, FLT3L, UM729, and SR1 for 15-25 days. In some embodiments, the method of differentiating HPs into NK comprises contacting a population of HP cells with a xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1. In some embodiments, a method of differentiating HPs in NK cells comprises contacting a population of stem cells with a xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1, for 15 to 25 days.

In some embodiments, a method of differentiating stem cells into NK cells comprises (i) contacting a population of stem cells with a first xenogenic-free differentiation media comprising BMP4, FGF2, and VEGF to generate a population of mesoderm cells, (ii) contacting the population of mesoderm cells with a second xenogenic-free differentiation media comprising BMP4, FGF2, VEGF, SCF, LDL, TPO, and a PI3K inhibitor to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, and 5-100 ng/mL VEGF to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising BMP4, FGF2, and VEGF for 12-120 hours to generate a population of mesoderm cells, (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, and 5-100 ng/mL VEGF for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 M of an aryl hydrocarbon receptor antagonist for 15-25 days to generate NK cells.

In some embodiments, a method of differentiating stem cells into NK cells comprises (i) contacting a population of stem cells with a first xenogenic-free differentiation media comprising BMP4, FGF2, and VEGF to generate a population of mesoderm cells, (ii) contacting the population of mesoderm cells with a second xenogenic-free differentiation media comprising BMP4, FGF2, VEGF, SCF, LDL, TPO, and LY294002 to generate hematopoietic progenitors, and (iii) contacting the population of hematopoietic progenitor cells with a third xenogenic-free differentiation media comprising SCF, IL-7, IL-15, IL-12, FLT3L, UM729, and SR1 to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, and 5-100 ng/mL VEGF to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 M of UM729, and 1-10 μM of SR1 to generate NK cells.

In some embodiments, a method of differentiating stem cells into hematopoietic progenitors comprises (i) contacting a population of stem cells with a first xenogenic-free differentiation media comprising BMP4, FGF2, and VEGF for 12-120 hours to generate a population of mesoderm cells, (ii) contacting the population of mesoderm cells with a second xenogenic-free differentiation media comprising BMP4, FGF2, VEGF, SCF, LDL, TPO, and LY294002 for 2-20 days to generate hematopoietic progenitors, and (iii) contacting the population of hematopoietic progenitor cells with a third xenogenic-free differentiation media comprising SCF, IL-7, IL-15, IL-12, FLT3L, UM729, and SR1 for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, and 5-100 ng/mL VEGF for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1 for 15-25 days to generate NK cells.

In some embodiments, a method of differentiating stem cells into NK cells comprises (i) contacting a population of stem cells with a first xenogenic-free differentiation media comprising BMP4, FGF2, VEGF, and a ROCK inhibitor to generate a population of mesoderm cells, (ii) contacting the population of mesoderm cells with a second xenogenic-free differentiation media comprising BMP4, FGF2, VEGF, SCF, LDL, TPO, and a PI3K inhibitor to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising BMP4, FGF2, VEGF, a ROCK inhibitor for 12-120 hours to generate a population of mesoderm cells, (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 M of a pyrimido-indole derivative, and 1-10 μM of an aryl hydrocarbon receptor antagonist for 15-25 days to generate NK cells.

In some embodiments, a method of differentiating stem cells into NK cells comprises (i) contacting a population of stem cells with a first xenogenic-free differentiation media comprising BMP4, FGF2, VEGF, and a ROCK inhibitor to generate a population of mesoderm cells, (ii) contacting the population of mesoderm cells with a second xenogenic-free differentiation media comprising BMP4, FGF2, VEGF, SCF, LDL, TPO, and LY294002 to generate hematopoietic progenitors, and (iii) contacting the population of hematopoietic progenitor cells with a third xenogenic-free differentiation media comprising SCF, IL-7, IL-15, IL-12, FLT3L, UM729, and SR1 to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 IM of SR1 to generate NK cells.

In some embodiments, a method of differentiating stem cells into hematopoietic progenitors comprises (i) contacting a population of stem cells with a first xenogenic-free differentiation media comprising BMP4, FGF2, VEGF, and a ROCK inhibitor for 12-120 hours to generate a population of mesoderm cells, (ii) contacting the population of mesoderm cells with a second xenogenic-free differentiation media comprising BMP4, FGF2, VEGF, SCF, LDL, TPO, and LY294002 for 2-20 days to generate hematopoietic progenitors, and (iii) contacting the population of hematopoietic progenitor cells with a third xenogenic-free differentiation media comprising SCF, IL-7, IL-15, IL-12, FLT3L, UM729, and SR1 for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of a ROCK inhibitor for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1 for 15-25 days to generate NK cells.

In some embodiments, a method of differentiating stem cells into NK cells comprises (i) contacting a population of stem cells with a first xenogenic-free differentiation media comprising BMP4, FGF2, VEGF, and Y27632 to generate a population of mesoderm cells, (ii) contacting the population of mesoderm cells with a second xenogenic-free differentiation media comprising BMP4, FGF2, VEGF, SCF, LDL, TPO, and a PI3K inhibitor to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist to generate NK cells.

In some embodiments, a method of differentiating stem cells into hematopoietic progenitors comprises (i) contacting a population of stem cells with a first xenogenic-free differentiation media comprising BMP4, FGF2, VEGF, and Y27632 for 12-120 hours to generate a population of mesoderm cells, (ii) contacting the population of mesoderm cells with a second xenogenic-free differentiation media comprising BMP4, FGF2, VEGF, SCF, LDL, TPO, and a PI3K inhibitor for 2-20 days to generate hematopoietic progenitors, and (iii) contacting the population of hematopoietic progenitor cells with a third xenogenic-free differentiation media comprising SCF, IL-7, IL-15, IL-12, FLT3L, a pyrimido-indole derivative, and an aryl hydrocarbon receptor antagonist for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of a PI3K inhibitor for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 μM of a pyrimido-indole derivative, and 1-10 μM of an aryl hydrocarbon receptor antagonist for 15-25 days to generate NK cells.

In some embodiments, a method of differentiating stem cells into NK cells comprises (i) contacting a population of stem cells with a first xenogenic-free differentiation media comprising BMP4, FGF2, VEGF, and Y27632 to generate a population of mesoderm cells, (ii) contacting the population of mesoderm cells with a second xenogenic-free differentiation media comprising BMP4, FGF2, VEGF, SCF, LDL, TPO, and LY294002 to generate hematopoietic progenitors, and (iii) contacting the population of hematopoietic progenitor cells with a third xenogenic-free differentiation media comprising SCF, IL-7, IL-15, IL-12, FLT3L, UM729, and SR1 to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 to generate hematopoietic progenitors, and (iii) contacting the population of hematopoietic progenitor cells with a third xenogenic-free differentiation media comprising 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1 to generate NK cells.

In some embodiments, a method of differentiating stem cells into hematopoietic progenitors comprises (i) contacting a population of stem cells with a first xenogenic-free differentiation media comprising BMP4, FGF2, VEGF, and Y27632 for 12-120 hours to generate a population of mesoderm cells, (ii) contacting the population of mesoderm cells with a second xenogenic-free differentiation media comprising BMP4, FGF2, VEGF, SCF, LDL, TPO, and LY294002 for 2-20 days to generate hematopoietic progenitors, and (iii) contacting the population of hematopoietic progenitor cells with a third xenogenic-free differentiation media comprising SCF, IL-7, IL-15, IL-12, FLT3L, UM729, and SR1 for 15-25 days to generate NK cells.

In some embodiments, a method of differentiating stem cells into hematopoietic progenitors comprises (i) contacting a population of stem cells with a first xenogenic-free differentiation media comprising 5-50 ng/mL BMP4, 5-50 ng/mL FGF2, 5-100 ng/mL VEGF, and 1-20 μM of Y27632 for 12-120 hours to generate a population of mesoderm cells, (ii) contacting the population of mesoderm cells with a second xenogenic-free differentiation media comprising 5-50 ng/mL BMP4, 5-50 ng/mL FGF2, 5-100 ng/mL VEGF, about 100 ng/mL SCF, about 50 μg/mL LDL, about 100 ng/mL TPO, and 5-100 uM of LY294002 for 2-20 days to generate hematopoietic progenitors, and (iii) contacting the population of hematopoietic progenitor cells with a third xenogenic-free differentiation media 5-50 ng/mL SCF, 5-50 ng/mL IL-7, 5-100 ng/mL IL-15, 5-100 ng/mL IL-12, 5-100 ng/mL FLT3L, 1-10 μM of UM729, and 1-10 μM of SR1 for 15-25 days to generate NK cells.

### Characteristics of NK Cells

In some embodiments, the NK cells produced by the methods of the disclosure have improved or enhanced properties compared to NK cells produced by other methods.

In some embodiments, the NK cells produced by the methods of the disclosure have enhanced expansion. In some embodiments, the NK cells have at least a 50 fold expansion, at least a 100 fold expansion, at least a 150 fold expansion, at least a 200 fold expansion, at least a 250 fold expansion, at least a 300 fold expansion, at least a 350 fold expansion, at least a 400 fold expansion, at least a 450 fold expansion, at least a 500 fold expansion, at least a 550 fold expansion, or at least a 600 fold expansion compared to NK cells produced by other methods.

In some embodiments, the NK cells have between a 50 fold expansion to a 100 fold expansion, between a 100 fold expansion to a 150 fold expansion, between a 150 fold expansion to a 200 fold expansion, between a 200 fold expansion to a 250 fold expansion, between a 250 fold expansion to a 300 fold expansion, between a 300 fold expansion to a 350 fold expansion, between a 350 fold expansion to a 400 fold expansion, between a 400 fold expansion to a 450 fold expansion, between a 450 fold expansion to a 500 fold expansion, between a 500 fold expansion to a 550 fold expansion, or between a 550 fold expansion to a 600 fold expansion compared to NK cells produced by other methods.

In some embodiments, the differentiated NK cells produced by the methods of the disclosure reduce tumor cell growth. In some embodiments, the differentiated NK cells reduce a tumor cell growth at least 50%, at least 60%, at least 70%, at least 80%, at least 90%, or 100%.

In some embodiments, the differentiated NK cells reduce a tumor cell growth between 50% to 60%, between 60% to 70%, between 70% to 80%, between 80%/0 to 90%, or between 90% to 100%.

In some embodiments, the mature NK cells produced by the methods of the disclosure reduce tumor cell growth. In some embodiments, the mature NK cells reduce tumor cell growth at least 50%, at least 60%, at least 70%, at least 80%, at least 90%, or 100%,

In some embodiments, the mature NK cells reduce tumor cell growth between 50% to 60%, between 60% to 70%, between 70% to 80%, between 80% to 90%, or between 90% to 100%.

In some embodiments, the methods of the disclosure produce a differentiated NK cell population. In some embodiments, the methods of the disclosure produce a differentiated NK cell population that is at least 40%, at least 50%, at least 60%, at least 70%, at least 80%, at least 90% or 100% of CD34+CD43+CD45+ LFA1+ quadruple-positive cells.

In some embodiments, the methods of the disclosure produce a differentiated NK cell population that is between 40% to 50%, between 50% to 60%, between 60% to 70%, between 70% to 80%, between 80% to 90%, or between 90% to 100% of CD34+CD43+CD45+ LFA1+ quadruple-positive cells.

In some embodiments, the methods of the disclosure produce a mature NK cell population. In some embodiments, the methods of the disclosure produce a mature NK cell population that is at least 40%, at least 50%, at least 60%, at least 70%, at least 80%, at least 90% or 100% of CD34+CD43+CD45+ LFA1+ NKp46+ NKG2D+ LFA1+CD161-CD73− cells.

In some embodiments, the methods of the disclosure produce a mature NK cell population that is between 40% to 50%, between 50% to 60%, between 60% to 70%, between 70% to 80%, between 80% to 90%, or between 90% to 100% of CD34+CD43+CD45+ LFA1+ NKp46+ NKG2D+ LFA1+CD161-CD73− cells.

### Engineered Cells

In some embodiments, the cell populations described herein are genetically engineered. In some embodiments, the source cells are genetically engineered. In some embodiments, the mesoderm cells are genetically engineered. In some embodiments, the embryoid body cells are genetically engineered. In some embodiments, the hematopoietic progenitor cells are genetically engineered. In some embodiments, the differentiated NK cells are genetically engineered. In some embodiments, the mature NK cells are genetically engineered. In some embodiments, genetic engineering reduces expression of an endogenous gene. In some embodiments, genetic engineering increases expression of an endogenous gene.

In some embodiments, genetically engineering a cell comprises introducing foreign DNA into the cell. In some embodiments, the foreign DNA is a gene. In some embodiments, the foreign DNA alters expression of endogenous genes.

In some embodiments, genetic engineering comprises introducing RNA into the cell, such as interfering RNAs (RNAi), Double-stranded RNA (dsrna), small interfering RNAs (siRNAs), and/or microRNA (miRNA).

In some embodiments, genetic engineering comprises introducing DNA into the cell, such as a plasmid or a bacterial artificial chromosome (BAC).

In some embodiments, genetic engineering comprises introducing: (a) a fusion protein comprising a DNA-targeting protein and a nuclease or (b) an RNA-guided nuclease. For example, in some embodiments, the DNA-targeting protein or RNA-guided nuclease comprises a zinc finger protein (ZFP), a TAL protein, or a clustered regularly interspaced short palindromic nucleic acid (CRISPR) specific for the gene. In some embodiments, the disruption comprises introducing a zinc finger nuclease (ZFN), a TAL-effector nuclease (TALEN), or and a CRISPR-Cas9 combination that specifically binds to, recognizes, or hybridizes to the gene. In some embodiments, the introducing is carried out by introducing into the cell a nucleic acid comprising a sequence encoding the DNA-binding protein, DNA-binding nucleotide, and/or complex comprising the DNA-binding protein or DNA-binding nucleotide. In some embodiments, the nucleic acid is a viral vector.

In some embodiments, a genetically engineered cell described herein comprises a chimeric antigen receptor (CAR). In some embodiments, a genetically engineered stem cell comprises a CAR. In some embodiments, a genetically engineered hematopoietic progenitor comprises a CAR. In some embodiments, a genetically engineered NK cell comprises a CAR.

In some embodiments, a genetically engineered cell described herein comprises a rapamycin-activated cytokine receptor (RACR). In some embodiments, a genetically engineered stem cell comprises a RACR. In some embodiments, a genetically engineered hematopoietic progenitor comprises a RACR. In some embodiments, a genetically engineered NK cell comprises a RACR.

In some embodiments, a genetically engineered cell described herein comprises a CAR and a RACR. In some embodiments, a genetically engineered stem cell comprises a CAR and a RACR. In some embodiments, a genetically engineered hematopoietic progenitor comprises a CAR and a RACR. In some embodiments, a genetically engineered NK cell comprises a CAR and a RACR.

In some embodiments, the genetically engineered NK cells may comprise an inactivating mutation. In some embodiments, an inactivating mutation is a nonsense mutation. In some embodiments, the nonsense mutation is a premature stop codon. In some embodiments, an inactivating mutation is a missense mutation.

### Synthetic Cytokine Receptor Complex

In some embodiments, a cell described herein is genetically engineered to express a synthetic cytokine receptor. In some embodiments, a synthetic cytokine receptor comprises a synthetic gamma chain and a synthetic beta chain, each comprising a dimerization domain. The dimerization domains controllable dimerize in the present of a non-physiological ligand, thereby activating signaling the synthetic cytokine receptor.

The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain) and N- or C-terminal to the IL-2G intracellular domain.

The synthetic beta chain polypeptide comprises a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2RB or IL-7RB intracellular domain).

The non-physiological ligand may activate the synthetic cytokine receptor in the cytotoxic innate lymphoid cells to induce expansion and/or activation of the engineered cytotoxic innate lymphoid cells. In a preferred embodiment, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the NK cells to induce expansion of the NK cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of NK cells compared to uninduced cells.

In some embodiments, the NK cells increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges.

### Intracellular Domain

In some embodiments, the intracellular signaling domain of the first transmembrane receptor protein comprises an interleukin-2 receptor subunit gamma (IL2Rg) domain. In some embodiments, the IL2Rg domain comprises the sequence set forth in SEQ ID NO: 1. In some embodiments, the IL2Rg Common Gamma Chain Intracellular domain has at least 80% amino acid identity, at least 85% amino acid identity, at least 90% amino acid identity, at least 95% amino acid identity, or 100% amino acid identity to SEQ ID NO: 1.

The sequence of a IL2RG Common Gamma Chain Intracellular domain is set forth in SEQ ID NO: 1:

ERTMPRIPTLKNLEDLVTEYHGNFSAWSGVSKGLAESLQPDYSERLCL
VSEIPPKGGALGEGPGASPCNQHSPYWAPPCYTLKPET.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL2RB) intracellular domain. In some embodiments, the IL2RB intracellular domain comprises the sequence set forth in SEQ ID NO: 2. In some embodiments, the IL2RB intracellular domain has at least 80% amino acid identity, at least 85% amino acid identity, at least 90% amino acid identity, at least 95% amino acid identity, or 100% amino acid identity to SEQ ID NO: 2.

The sequence of a IL2RB intracellular domain is set forth in SEQ ID NO: 2.

NCRNTGPWLKKVLKCNTPDPSKFFSQLSSEHGGDVQKWLSSPFPSSSF
SPGGLAPEISPLEVLERDKVTQLLLQQDKVPEPASLSSNHSLTSCFTN
QGYFFFHLPDALEIEACQVYFTYDPYSEEDPDEGVAGAPTGSSPQPLQ
PLSGEDDAYCTFPSRDDLLLFSPSLLGGPSPPSTAPGGSGAGEERMPP
SLQERVPRDWDPQPLGPPTPGVPDLVDFQPPPELVLREAGEEVPDAGP
REGVSFPWSRPPGQGEFRALNARLPLNTDAYLSLQELQGQDPTHLV

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL7RB) intracellular domain. In some embodiments, the IL7RB intracellular domain comprises the sequence set forth in SEQ ID NO: 3. In some embodiments, the IL7RB intracellular domain has at least 80% amino acid identity, at least 85% amino acid identity, at least 90% amino acid identity, at least 95% amino acid identity, or 100/6 amino acid identity to SEQ ID NO: 3.

The sequence of a IL7RB intracellular domain is set forth in SEQ ID NO: 3:

KKRIKPIVWPSLPDHKKTLEHLCKKPRKNLNVSFNPESFLDCQIHRVD
DIQARDEVEGFLQDTFPQQLEESEKQRLGGDVQSPNCPSEDVVITPES
FGRDSSLTCLAGNVSACDAPILSSSRSLDCRESGKNGPHVYQDLLLSL
GTTNSTLPPPFSLQSGILTLNPVAQGQPILTSLGSNQEBAYVTMSSFY
QNQ

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL21RB) intracellular domain. In some embodiments, the IL21RB intracellular domain comprises the sequence set forth in SEQ ID NO: 4. In some embodiments, the IL21RB intracellular domain has at least 80% amino acid identity, at least 85% amino acid identity, at least 90% amino acid identity, at least 95% amino acid identity, or 100% amino acid identity to SEQ ID NO: 4.

The sequence of a IL21RB intracellular domain is set forth in SEQ ID NO: 4:

SLKTHPLWRLWKKIWAVPSPERFFMPLYKGCSGDFKKWVGAPFTGSSL
ELGPWSPEVPSTLEVYSCHPPRSPAKRLQLTELQEPAELVESDGVPKP
SFWPTAQNSGGSAYSEERDRPYGLVSIDTVTVLDAEGPCTWPCSCEDD
GYPALDLDAGLEPSPGLEDPLLDAGTTVLSCGCVSAGSPGLGGPLGSL
LDRLKPPLADGEDWAGGLPWGGRSPGGVSESEAGSPLAGLDMDTFDSG
FVGSDCSSPVECDFTSPGDEGPPRSYLRQWVVIPPPLSSPGPQAS

### Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog. The FRB domain may comprise a polypeptide sequence at least 80%, at least 90%, at least 95%, at least 96%, at least 97%, at least 98%, at least 99%, or 100% identical to SEQ ID NO: 6 or SEQ ID NO:7. The FKBP domain may comprise a polypeptide sequence at least 80%, at least 90%, at least 95%, at least 96%, at least 97%, at least 98%, at least 99%, or 100% identical to SEQ ID NO: 5.

The sequence of an illustrative FKBP domain is set forth in SEQ ID NO: 5:

GVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHAT
LVFDVELLKLE

The sequence of an illustrative FRB domain is set forth in SEQ ID NO: 6: ILWHEMWHEGLEEASRLYFGERNVKGMFEVLEPLHAMMERGPQTLKETSFNQAYG RDLMEAQEWCRKYMKSGNVKDLTQAWDLYYHVFRRISK

The sequence of variant FRB domain (FRB mutant domain) is set forth in SEQ ID NO: 7:

ILWHEMWHEGLEEASRLYFGERNVKGMFEVLEPLHAMMERGPQTLKET
SFNQAYGRDLMEAQEWCRKYMKSGNVKDLLQAWDLYYHVFRRISK

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) cyclophiliA (CypA); or
iii) gyrase B (CyrB);
with the corresponding non-physiological ligands being, respectively
i) FK1012, AP1510, AP1903, or AP20187;
ii) cyclosporin-A (CsA); or
iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYRI-like 1 (PYL1) and abscisic acid insensitive 1 (ABI1).

### Transmembrane Domains

The transmembrane domain is the sequence of the synthetic cytokine receptor that spans the membrane. The transmembrane domain may comprise a hydrophobic alpha helix. In some embodiments, the transmembrane domain is derived from a human protein.

The sequence of a transmembrane (TM) domain is shown as SEQ ID NO: 8:

VVISVGSMGLIISLLCVYFWL

The sequence of a TM domain is shown as SEQ ID NO: 9:

VAVAGCVFLLISVLLLSGL

The sequence of TM domain is shown as SEQ ID NO: 10:

PILLTISILSFFSVALLVILACVLW

The sequence of a TM domain is shown as SEQ ID NO: 11:

GWNPHLLLLLLLVIVFIPAFW

The sequence of a CD8a signal sequence is shown as SEQ ID NO: 12:

MALPVTALLLPLALLLHAARP

### Non-Physiological Ligand

In various embodiments of the compositions and methods of the disclosure, the system comprises a non-physiological ligand. Illustrative small molecules useful as ligands include, without limitation: rapamycin, fluorescein, fluorescein isothiocyanate (FITC), 4-[(6-methylpyrazin-2-yl) oxy]benzoic acid (aMPOB), folate, rhodamine, acetazolamide, and a CA9 ligand.

In some embodiments, the synthetic cytokine receptor is activated by a ligand. In some embodiments, the ligand is a non-physiological ligand.

In some embodiments, the non-physiological ligand is a rapalog.

In some embodiments, the non-physiological ligand is rapamycin.

In some embodiments, the non-physiological ligand is AP21967.

In some embodiments, the non-physiological ligand is FK506.

In some embodiments, the non-physiological ligand is FK1012. In some embodiments, the non-physiological ligand is AP1510. In some embodiments, the non-physiological ligand is AP1903. In some embodiments, the non-physiological ligand is AP20187. In some embodiments, the non-physiological ligand is cyclosporin-A (CsA). In some embodiments, the non-physiological ligand is coumermycin.

In some embodiments, the synthetic cytokine receptor complex activated by folate, fluorescein, aMPOB, acetazolamide, a CA9 ligand, tacrolimus, rapamycin, a rapalog (a rapamycin analog), CD28 ligand, poly(his) tag, Strep-tag, FLAG-tag, VS-tag, Myc-tag, HA-tag, NE-tag, biotin, digoxigenin, dinitrophenol, or a derivative thereof.

In some embodiments, the non-physiological ligand may be an inorganic or organic compound that is less than 1000 Daltons.

In some embodiments, the ligand may be rapamycin or a rapamycin analog (rapalog). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

Thus, in some embodiments, the rapalog is everolimus, novolimus, pimecrolimus, ridaforolimus, tacrolimus, temsirolimus, umirolimus, zotarolimus, Temsirolimus (CCI-779), C20-methallylrapamycin, C16-(S)-3-methylindolerapamycin, C16-(S)-3-methylindolerapamycin (C16-iRap), AP21967 (A/C Heterodimerizer, Takara Bio®), sodium mycophenolic acid, benidipine hydrochloride, rapamine, AP23573 (Ridaforolimus), AP1903 (Rimiducid), or metabolites, derivatives, and/or combinations thereof.

In some embodiments, the ligand comprises FK1012 (a semisynthetic dimer of FK506), tacrolimus (FK506), FKCsA (a composite of FK506 and cyclosporine), rapamycin, coumermycin, gibberellin, HaXS dimerizer (chemical dimerizers of HaloTag and SNAP-tag), TMP-HTag (trimethoprim haloenzyme protein dimerizer), or ABT-737 or functional derivatives thereof.

In some embodiments, the non-physiological ligand is present or provided in an amount from 0 nM to 1000 nM such as, e.g., 0.05 nM, 0.1 nM, 0.5. nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, or 1000 nM, or an amount that is within a range defined by any two of the aforementioned amounts.

In some embodiments, the non-physiological ligand is AP21967 and is present or provided at 10 nM. In some embodiments, the non-physiological ligand is AP21967 and is present or provided at 20 nM. In some embodiments, the non-physiological ligand is AP21967 and is present or provided at 50 nM. In some embodiments, the non-physiological ligand is AP21967 and is present or provided at 100 nM.

In some embodiments, the non-physiological ligand is rapamycin and is present or provided at 1 nM. In some embodiments, the non-physiological ligand is rapamycin and is present or provided at 10 nM. In some embodiments, the non-physiological ligand is rapamycin and is present or provided at 20 nM. In some embodiments, the non-physiological ligand is rapamycin and is present or provided at 50 nM.

In some embodiments, the non-physiological ligand is a rapalog and is present or provided at 1 nM. In some embodiments, the non-physiological ligand is a rapalog and is present or provided at 10 nM. In some embodiments, the non-physiological ligand is a rapalog and is present or provided at 20 nM. In some embodiments, the non-physiological ligand is a rapalog and is present or provided at 50 nM. In some embodiments, the non-physiological ligand is a rapalog and is present or provided at 100 nM.

In some embodiments, the non-physiological ligand is present or provided at 1 nM. In some embodiments, the non-physiological ligand is present or provided at 10 nM. In some embodiments, the non-physiological ligand is present or provided at 100 nM. In some embodiments, the non-physiological ligand is present or provided at 1000 nM.

### Cytosolic FRB

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the transduced cells and promote consistent activation of transduced cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. The cytosolic FRB domain may comprise a polypeptide sequence at least 80%, at least 90%, at least 95%, at least 96%, at least 97%, at least 98%, at least 99%, or 100% identical to SEQ ID NO: 6 or SEQ ID NO: 7. FRB domain may be a naked FRB domain consisting essentially of a polypeptide having a polypeptide sequence at least 80%, at least 90%, at least 95%, at least 96%, at least 97%, at least 98%, at least 99%, or 100% identical to SEQ ID NO: 6 or SEQ ID NO: 7. Advantageously, the cytosolic FRB confers resistance to the immunosuppressive effect of the non-physiological ligand (e.g., rapamycin or rapalog).

### Chimeric Antigen Receptor

In some embodiments, a cell described herein is genetically engineered to express a chimeric antigen receptor (CAR).

In some embodiments, the disclosure contemplates a CAR system for use in the treatment of subjects with cancer. In some embodiments, the NK cells of the disclosure comprise a CAR sequence (CAR-NK cells).

In some embodiments, NK cells are engineered to express CAR constructs by transfecting a population of cells with an expression vector encoding the CAR construct. Illustrative examples of populations of cells that may be transfected include HSCs, blood progenitor cells, common lymphoid progenitor cells, or NK cells. Appropriate means for preparing a transduced population of NK cells expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples. In some embodiments, any of the transduction methods contemplated in the disclosure may be used to generate CAR-expressing NK cells.

### Targeting Agents for CARs

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFv's for innumerable haptens are known in the art or can be generated by conventional methods routinely. Accordingly, the present disclosure contemplates using any known hapten-binding domain.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR-NK cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In some embodiments, the CAR system of the disclosure makes use of CARs that target a moiety that is not produced or expressed by cells of the subject being treated. This CAR system thus allows for focused targeting of the NK cells to target cells, such as cancer cells. By administration of a small conjugate molecule along with the CAR-expressing NK cells, the NK cell response can be targeted to only those cells expressing the tumor receptor, thereby reducing off-target toxicity, and the activation of NK cells can be more easily controlled due to the rapid clearance of the small conjugate molecule. As an added advantage, the CAR-expressing NK cells can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

In one embodiment, the disclosure provides an illustration of this conjugate molecule/CAR system.

In some embodiments, the CAR system of the disclosure utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (KD=14 nM; see Kularatne, S. A. et al., Mol Pharm. 2009. 6(3):780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27(4):655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptidoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

An illustrative conjugate of the disclosure is FITC-Folate

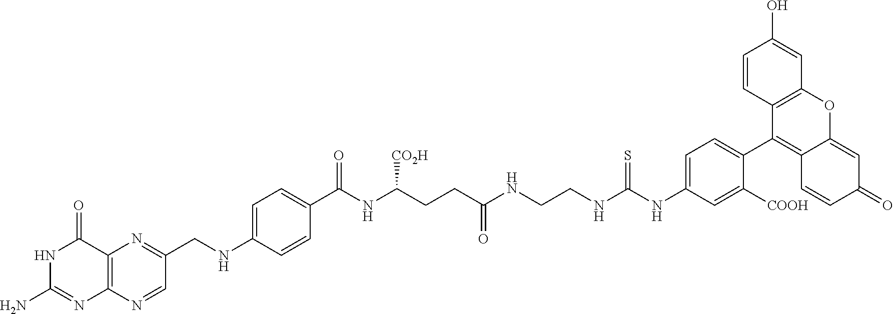 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

An illustrative conjugate of the disclosure is FITC-CA9

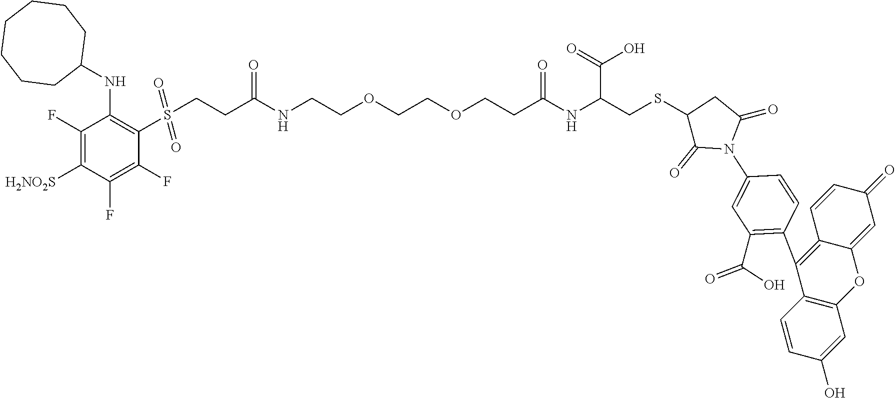 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

Illustrative conjugates of the disclosure include the following molecules: FITC-(PEG)↔12-Folate, FITC-(PEG)↔20-Folate, FITC-(PEG)↔108-Folate, FITC-DUPA, FITC-(PEG)12-DUPA, FITC-CCK2R ligand, FITC-(PEG)12-CCK2R ligand, FITC-(PEG)11-NK1R ligand and FITC-(PEG)2-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 μM or 10 μM, even more preferably at least about 100 μM.

Examples of conjugates and methods of making them are provided in U.S. patent applications US 2017/0290900, US 2019/0091308, and US 2020/0023009, all of which are incorporated herein by reference.

### CAR Constructs

In some embodiments, the binding portion of the CAR can be, for example, a single chain fragment variable region (scFv) of an antibody, a Fab, Fv, Fc, or F(ab′)2 fragment, and the like. The use of unaltered (i.e., full size) antibodies, such as IgG, IgM, IgA, IgD or IgE, in the CAR or as the CAR is excluded from the scope of the invention.

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina, L. et al., Eur J Immunol. 1996. 26(10):2304-9); CD137 (4-1BB), a member of the tumor necrosis factor (TNF) receptor family (see, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments, the CAR constructs comprise two co-stimulation domains. While the particular combinations include all possible variations of the four noted domains, specific examples include: 1) CD28+CD137 (4-1BB) and 2) CD28+CD134 (OX40).

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. Suitable activation signaling domains include the CD3ζ chain and Fc receptor γ. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

Illustrative CAR constructs suitable for CAR-NK cells are provided below:
(1) scFv-CD8TM-4-1BBIC-CD3ζs (see, e.g., Liu E, Tong Y, Dotti G, et al., Leukemia. 2018; 32: 520-531);
(2) scFv-CD28TM+IC-CD3ζs (see, e.g., Han J, Chu J, Keung C W et al., Sci Rep. 2015; 5: 11483; Kruschinski A, Moosmann A, Poschke I et al., Proc Natl Acad Sci USA. 2008; 105: 17481-17486; and Chu J, Deng Y, Benson D M et al., Leukemia. 2014; 28: 917-927);
(3) scFv-DAP12TM+IC (see, e.g., Muller N, Michen S, Tietze S et al., J Immunother. 2015; 38: 197-210);
(4) scFv-CD8TM-2B4IC-CD3ζs (see, e.g., Xu Y, Liu Q, Zhong M et al., J Hematol Oncol. 2019; 12: 49);
(5) scFv-2B4TM+IC-CD3ζs (see, e.g., Altvater B, Landmeier S, Pscherer S et al., Clin Cancer Res. 2009; 15: 4857-4866);
(6) scFv-CD28TM+IC4-1BBIC-CD3ζs (see, e.g., Kloss S, Oberschmidt O, Morgan M et al., Hum Gene Ther. 2017; 28: 897-913);
(7) scFv-CD16TM-2B4IC-CD3ζs (see, e.g., Li Y, Hermanson D L, Moriarity B S Kaufman D S, Cell Stem Cell. 2018; 23: 181-192);
(8) scFv-NKp44TM-DAP10IC-CD3ζs (see, e.g., Li Y, Hermanson D L, Moriarity B S Kaufman D S, Cell Stem Cell. 2018; 23: 181-192);
(9) scFv-NKp46TM-2B4IC-CD3ζs (see, e.g., Li Y, Hermanson D L, Moriarity B S Kaufman D S, Cell Stem Cell. 2018; 23: 181-192);
(10) scFv-NKG2DTM-2B41C-CD3ζs (see, e.g., Li Y, Hermanson D L, Moriarity B S Kaufman D S, Cell Stem Cell. 2018; 23: 181-192);
(11) scFv-NKG2DTM-4-1BBIC-CD3ζs (see, e.g., Li Y, Hermanson D L, Moriarity B S Kaufman D S, Cell Stem Cell. 2018; 23: 181-192);
(12) scFv-NKG2DTM-2B4IC-DAP12IC-CD3ζs (see, e.g., Li Y, Hermanson D L, Moriarity B S Kaufman D S, Cell Stem Cell. 2018; 23: 181-192);
(13) scFv-NKG2DTM-2B4IC-DAP10IC-CD3ζs (see, e.g., Li Y, Hermanson D L, Moriarity B S Kaufman D S, Cell Stem Cell. 2018; 23: 181-192);
(14) scFv-NKG2DTM-4-1BBIC-2B4IC-CD3ζS (see, e.g., Li Y, Hermanson D L, Moriarity B S Kaufman D S, Cell Stem Cell. 2018; 23: 181-192); and
(15) scFv-NKG2DTM-CD3ζS (see, e.g., Li Y, Hermanson D L, Moriarity B S Kaufman D S, Cell Stem Cell. 2018; 23: 181-192).

An illustrative CAR of the disclosure is shown in FIG. 8 where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD3ζ.

An illustrative nucleotide sequence encoding a CAR may comprise SEQ ID NO: 13:

(SEQ ID NO: 13)
ATGGCCTTACCAGTGACCGCCTTGCTCCTGCCGCTGGCCTTGCTGCTCCA
CGCCGCCAGGCCGGATGTCGTGATGACCCAGACCCCCCTCAGCCTCCCAGTGTCC
CTCGGTGACCAGGCTTCTATTAGTTGCAGATCCAGCCAGTCCCTCGTGCACTCTA
ACGGTAATACCTACCTGAGATGGTATCTCCAGAAGCCCGGACAGAGCCCTAAGG
TGCTGATCTACAAAGTCTCCAACCGGGTGTCTGGAGTCCCTGACCGCTTCTCAGG
GAGCGGTTCCGGCACCGACTTCACCCTGAAGATCAACCGGGTGGAGGCCGAAGA
CCTCGGCGTCTATTTCTGCTCTCAGAGTACACATGTGCCCTGGACCTTCGGCGGA
GGGACCAAGCTGGAGATCAAAAGCTCCGCAGACGATGCCAAGAAAGATGCCGCT
AAGAAAGACGATGCTAAGAAAGACGATGCAAAGAAAGACGGTGGCGTGAAGCT
GGATGAAACCGGAGGAGGTCTCGTCCAGCCAGGAGGAGCCATGAAGCTGAGTTG
CGTGACCAGCGGATTCACCTTTGGGCACTACTGGATGAACTGGGTGCGACAGTCC
CCAGAGAAGGGGCTCGAATGGGTCGCTCAGTTCAGGAACAAACCCTACAATTAT
GAGACATACTATTCAGACAGCGTGAAGGGCAGGTTTACTATCAGTAGAGACGAT
TCCAAATCTAGCGTGTACCTGCAGATGAACAATCTCAGGGTCGAAGATACAGGC
ATCTACTATTGCACAGGGGCATCCTATGGTATGGAGTATCTCGGTCAGGGGACAA
GCGTCACAGTCAGTTTCGTGCCGGTCTTCCTGCCAGCGAAGCCCACCACGACGCC
AGCGCCGCGACCACCAACACCGGCGCCCACCATCGCGTCGCAGCCCCTGTCCCT
GCGCCCAGAGGCGTGCCGGCCAGCGGGGGGGGCGCAGTGCACACGAGGGGGC
TGGACTTCGCCTGTGATATCTACATCTGGGCGCCCTTGGCCGGGACTTGTGGGGT
CCTTCTCCTGTCACTGGTTATCACCCTTTACTGCAACCACAGGAACCGTTTCTCTG
TTGTTAAACGGGGCAGAAAGAAACTCCTGTATATATTCAAACAACCATTTATGAG
ACCAGTACAAACTACTCAAGAGGAAGATGGCTGTAGCTGCCGATTTCCAGAAGA
AGAAGAAGGAGGATGTGAACTGAGAGTGAAGTTCAGCAGGAGCGCAGACGCCC
CCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGG
GAAAGCCGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGGACAC
CTACGACGCCCTTCACATGCAGGCCCTGCCCCCTCGCTAA

An illustrative CAR amino acid sequence may comprise SEQ ID NO: 14:

(SEQ ID NO: 14)
MALPVTALLLPLALLLHAARPDVVMTQTPLSLPVSLGDQASISCRSSQSLVH
SNGNTYLRWYLOKPGQSPKVLIYKVSNRVSGVPDRFSGSGSGTDFTLKINRVEAEDL
GVYFCSQSTHVPWTFGGGTKLEIKSSADDAKKDAAKKDDAKKDDAKKDGGVKLDE
TGGGLVQPGGAMKLSCVTSGFTFGHYWMNWVRQSPEKGLEWVAQFRNKPYNYET
YYSDSVKGRFTISRDDSKSSVYLQMNNLRVEDTGIYYCTGASYGMEYLGQGTSVTV
SFVPVFLPAKPTTTPAPRPPTPAPTIASQPLSLRPEACRPAAGGAVHTRGLDFACDIYI
WAPLAGTCGVLLLSLVITLYCNHRNRFSVVKRGRKKLLYIFKQPFMRPVQTTQEEDG
CSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTA
TKDTYDALHMQALPPR

An illustrative nucleotide insert may comprise SEQ ID NO: 15:

(SEQ ID NO: 15)
GCCACCATGGCCTTACCAGTGACCGCCTTGCTCCTGCCGCTGGCCTTGCTGCTCC
ACGCCGCCAGGCCGGATGTCGTGATGACCCAGACCCCCCTCAGCCTCCCAGTGTC
CCTCGGTGACCAGGCTTCTATTAGTTGCAGATCCAGCCAGTCCCTCGTGCACTCT
AACGGTAATACCTACCTGAGATGGTATCTCCAGAAGCCCGGACAGAGCCCTAAG
GTGCTGATCTACAAAGTCTCCAACCGGGTGTCTGGAGTCCCTGACCGCTTCTCAG
GGAGCGGTTCCGGCACCGACTTCACCCTGAAGATCAACCGGGTGGAGGCCGAAG
ACCTCGGCGTCTATTTCTGCTCTCAGAGTACACATGTGCCCTGGACCTTCGGCGG
AGGGACCAAGCTGGAGATCAAAAGCTCCGCAGACGATGCCAAGAAAGATGCCG
CTAAGAAAGACGATGCTAAGAAAGACGATGCAAAGAAAGACGGTGGCGTGAAG
CTGGATGAAACCGGAGGAGGTCTCGTCCAGCCAGGAGGAGCCATGAAGCTGAGT
TGCGTGACCAGCGGATTCACCTTTGGGCACTACTGGATGAACTGGGTGCGACAGT
CCCCAGAGAAGGGGCTCGAATGGGTCGCTCAGTTCAGGAACAAACCCTACAATT
ATGAGACATACTATTCAGACAGCGTGAAGGGCAGGTTTACTATCAGTAGAGACG
ATTCCAAATCTAGCGTGTACCTGCAGATGAACAATCTCAGGGTCGAAGATACAG
GCATCTACTATTGCACAGGGGCATCCTATGGTATGGAGTATCTCGGTCAGGGGAC
AAGCGTCACAGTCAGTTTCGTGCCGGTCTTCCTGCCAGCGAAGCCCACCACGACG
CCAGCGCCGCGACCACCAACACCGGCGCCCACCATCGCGTCGCAGCCCCTGTCC
CTGCGCCCAGAGGCGTGCCGGCCAGCGGGGGGGGCGCAGTGCACACGAGGGG
GCTGGACTTCGCCTGTGATATCTACATCTGGGCGCCCTTGGCCGGGACTTGTGGG
GTCCTTCTCCTGTCACTGGTTATCACCCTTTACTGCAACCACAGGAACCGTTTCTC
TGTTGTTAAACGGGGCAGAAAGAAACTCCTGTATATATTCAAACAACCATTTATG
AGACCAGTACAAACTACTCAAGAGGAAGATGGCTGTAGCTGCCGATTTCCAGAA
GAAGAAGAAGGAGGATGTGAACTGAGAGTGAAGTTCAGCAGGAGCGCAGACGC
CCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGG
GGGAAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGCCGG
AGGGGCAAGGGGCACGATGGCCTTTACCAGGGTCTCAGTACAGCCACCAAGGAC
ACCTACGACGCCCTTCACATGCAGGCCCTGCCCCCTCGCTAA

In various embodiments, CAR-expressing cells comprising the nucleic acid of SEQ ID NO: 13 or 15 are provided. In some embodiments, a chimeric antigen receptor polypeptide comprising SEQ ID NO: 14 is contemplated. In some embodiments, a vector is contemplated comprising SEQ ID NO: 13 or 15. In some embodiments, a lentiviral vector is contemplated comprising SEQ ID NO: 13 or 15. In some embodiments, SEQ ID NO: 14 can comprise or consist of human or humanized amino acid sequences.

In some embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 13, SEQ ID NO: 14, or SEQ ID NO: 15 are contemplated.

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 μM, or 10 μM, preferably at least about 100 μM, 1 fM or 10 fM, even more preferably at least about 100 fM.

### Therapeutic Compositions

In some embodiments, the present disclosure provides a composition comprising one or more cell populations.

In some embodiments, the composition comprises a population of differentiated NK cells. In some embodiments, the composition comprises a population of differentiated NK cells that is at least 40%, at least 50%, at least 60%, at least 70%, at least 80%, at least 90% or 100% of CD34+CD43+CD45+ LFA1+ quadruple-positive cells.

In some embodiments, the composition comprises a population of differentiated NK cells that is between 40% to 50%, between 50% to 60%, between 60% to 70%, between 70% to 80%, between 80% to 90%, or between 90% to 100% of CD34+CD43+CD45+ LFA1+ quadruple-positive cells.

In some embodiments, the composition comprises a population of mature NK cells. In some embodiments, the composition comprises a population of mature NK cells that is at least 40%, at least 50%, at least 60%, at least 70%, at least 80%, at least 90% or 100% of CD34+CD43+CD45+ LFA1+ NKp46+ NKG2D+ LFA1+CD161-CD73− cells.

In some embodiments, the composition comprises a population of mature NK cells that is between 40% to 50%, between 50% to 60%, between 60% to 70%, between 70% to 80%, between 80% to 90%, or between 90% to 100% of CD34+CD43+CD45+ LFA1+ NKp46+ NKG2D+ LFA1+CD161-CD73− cells.

### Methods of Treating

The present disclosure provides methods of treating a subject in need thereof with the compositions, therapeutic compositions, or cells, disclosed herein. In some embodiments, the disclosure provides a method of treating cancer and/or killing cancer cells in a subject, comprising administering a therapeutically effective amount of the disclosed cells to the subject.

In some embodiments, the cancer is a solid tumor, sarcoma, carcinoma, lymphoma, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitfs lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be used to treat cancer and/or kill cancer cells in a subject by administering a therapeutically effective amount of the cells according to any of the foregoing embodiments.

The present disclosure also provides a method of treating cancer and/or killing cancer cells in a subject, comprising administering the composition of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, an additional cancer therapy is provided, such as a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

### Modes of Administration and Dosing

In some embodiments, NK cells may be grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma.

In some embodiments, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation either with or without the agent that was used to promote activation.

The disclosed cells may be administered in a number of ways depending upon whether local or systemic treatment is desired.

In the case of adoptive cell therapy, methods for administration of cells for adoptive cell therapy are known and may be used in connection with the provided methods and compositions.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In an embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Illustrative parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritic, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing differentiated cells or transgenic differentiated cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 10³ to about 10⁹ cells per kg body weight including all integer values of cell numbers within those ranges.

### Kits

Also provided herein are kits, including the cells described herein, as well as written instructions for making and using the same. Thus, for example, provided herein is a kit including the cell population as described herein written instructions for making and using the same.

In some embodiments, a kit can have one or more additional therapeutic agents that can be administered simultaneously or in sequence with another component for a desired purpose, e.g., genome edition or cell therapy.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (such as associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

### Example 1: Development of Xenogenic-Free NK Cell Differentiation Method

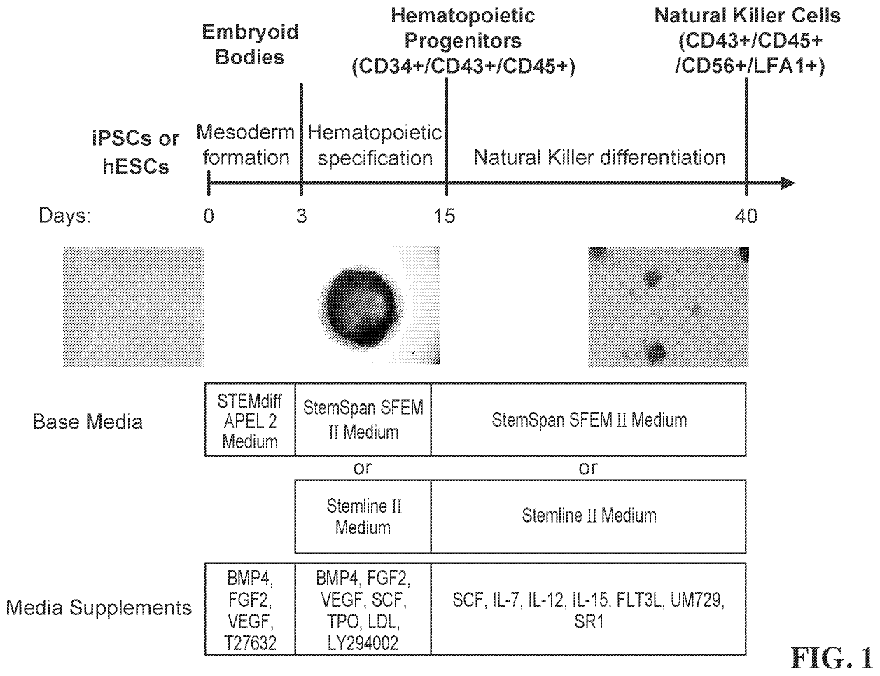

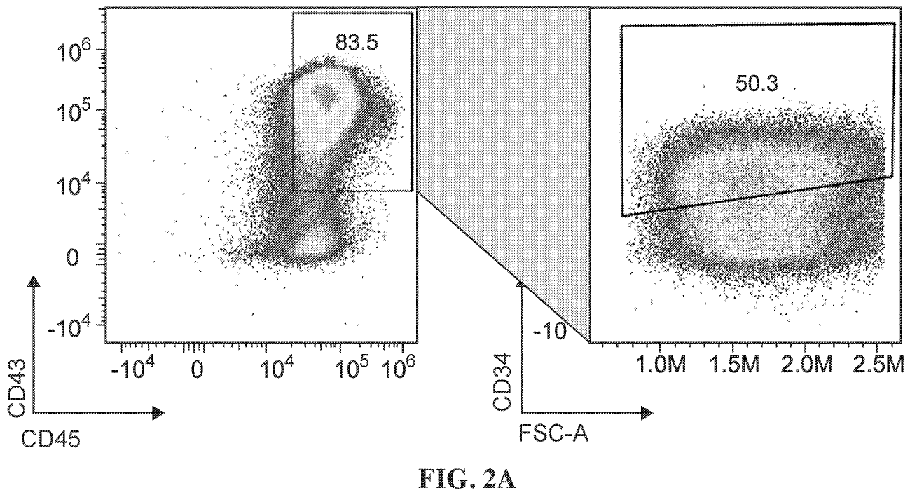

The purpose of this study was to develop a serum-free and xenogenic-free method of differentiating NK cells from stem cells. Specifically, undifferentiated human iPSCs were cultured in mTeSR Plus (STEMCELL Technologies) or E8 Flex (Gibco) media on hESC-qualified Matrigel (Corning) and routinely passaged using EDTA (Thermo Fisher Scientific). To generate embryoid bodies (EBs) from iPSCs, at day 0 a single-cell suspension of undifferentiated iPSCs was seeded into 96-well plates or Aggrewell microwell plates (STEMCELL technologies) (250-4000 cells/well) in STEMdiffAPEL 2 Medium (STEMCELL Technologies; media is fully defined, serum- and animal component-free) supplemented with Y27632 (1 to 20 uM), BMP4, FGF2 (both at 5 to 50 ng/mL), and VEGF (5 to 100 ng/mL) (days 0-3). To induce hematopoietic progenitor formation, EBs were cultured in StemSpan SFEM II Medium (STEMCELL Technologies; serum-free), Stemline II (Sigma-Aldrich; fully defined, serum- and animal component-free, GMP manufactured), B0 Medium (DMEM, F12, Human AB serum, 2-mercaptoethanol, ethanolamine, ascorbic acid, sodium selenite), CTS NK Xpander Medium (Gibco; serum-free and animal component-free medium), STEMdiff Hematopoietic—EB Basal Medium (STEMCELL Technologies; serum-free), or Hematopoietic Progenitor Expansion Medium XF (PromoCell; serum-free and xeno-free medium) supplemented with combinations of BMP4 and FGF2 (5 to 50 ng/mL), VEGF and SCF (up to 100 ng/mL), TPO (up to 25 ng/mL), LDL (up to 10 μg/mL), LY294002 (up to 4 uM), StemRegenin-1 (up to 1 uM), and UM729 (up to 1 uM) (days 3-15). To induce NK differentiation, cells were harvested (day 15) and cultured in StemSpan SFEM II Medium or Stemline II, supplemented with combinations of SCF (up to 20 ng/mL), IL-7 (up to 20 ng/mL), IL-15 (up to 20 ng/mL), FLT3L (up to 20 ng/mL), UM729 (up to 1 uM), and SR1 (up to 1 uM) and IL-12 (up to 20 ng/mL) (days 15-40). FIG. 1 provides a schematic of the differentiation method.

### Characterization of Hematopoetic Progenitors (HPs)

HPs were induced to form from iPSC-derived EBs as described in FIG. 1, with the following exceptions: StemSpan SFEM II media omitted LDL (days 3-15). To determine the percentage of HPs generated of the total cells present at day 15 of differentiation, flow cytometry analysis was performed, gating cells to quantify percentage triple-positive for the HP markers CD34/CD43/CD45.

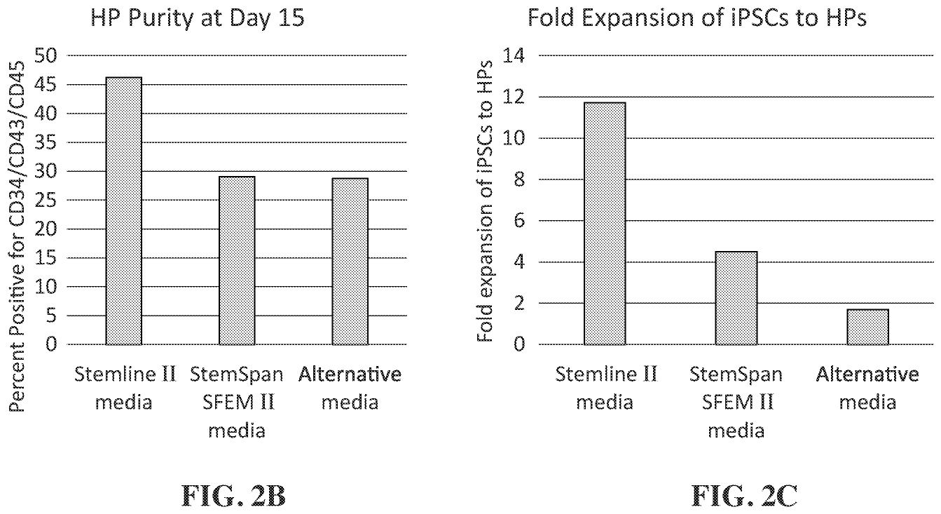

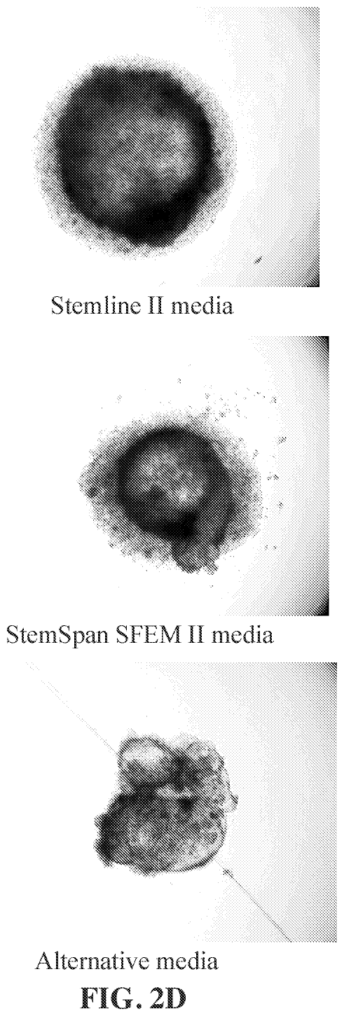

FIG. 2A shows results from cells cultured in Stemline media. High HP purity was observed, ranging from 29-46% of all cells being triple-positive for CD34/CD43/CD45 when either Stemline II or StemSpan SFEM II media were used (FIG. 2B). High yields of HPs were observed, ranging from 4.5 to 11.7-fold expansions of HPs at day 15 relative to iPSCs seeded at day 0 for StemSpan SFEM II or Stemline II media, respectively, compared to 1.7-fold expansion with an alternative media (FIG. 2C). Representative brightfield microscope images are shown of the EBs prior to HP harvesting on day 15 (FIG. 2D).

### Characterization of iNK Cells

iNKs were induced to form from iPSC-derived HPs as described in FIG. 1 (days 15-40). To determine the percentage of iNKs generated of the total cells present at day 40 of differentiation, flow cytometry analysis was performed, gating cells to quantify percentage positive for four NK markers, CD43/CD45/CD56/LFA1.

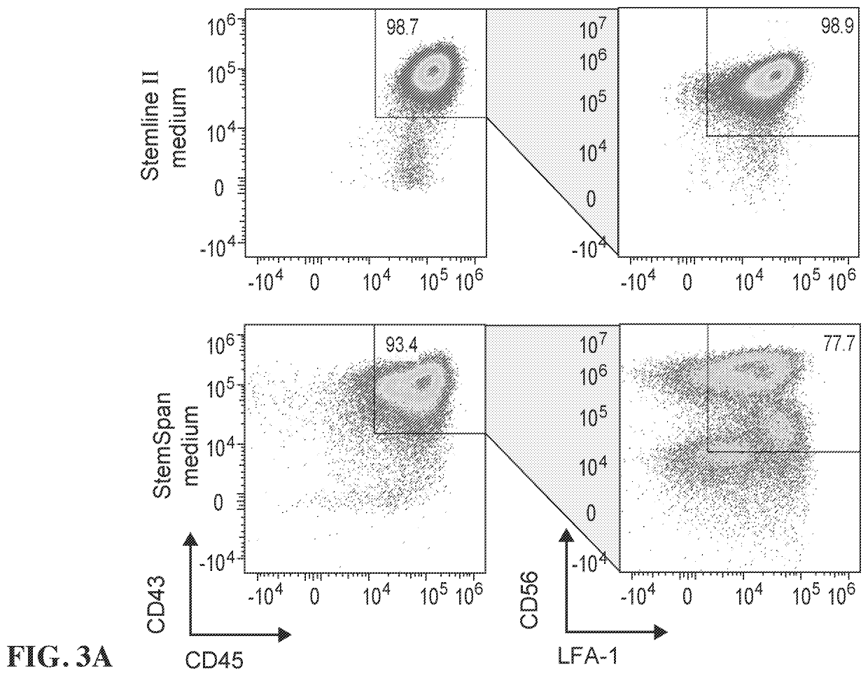

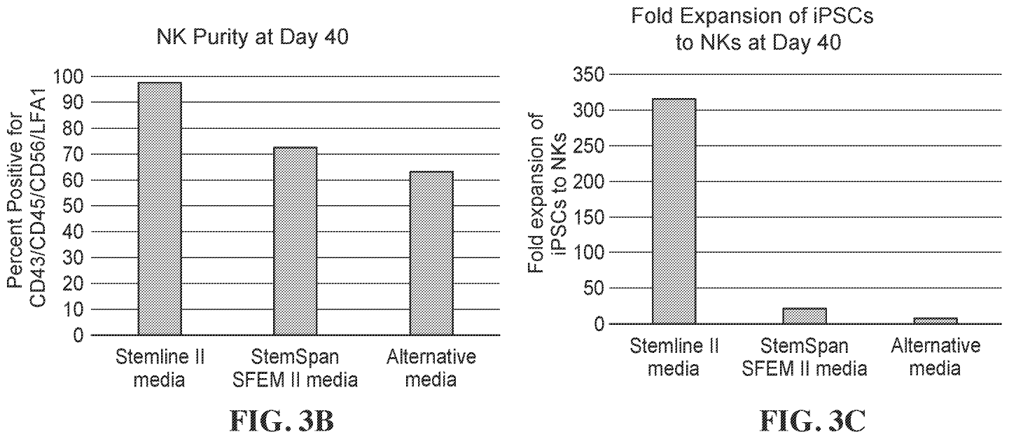

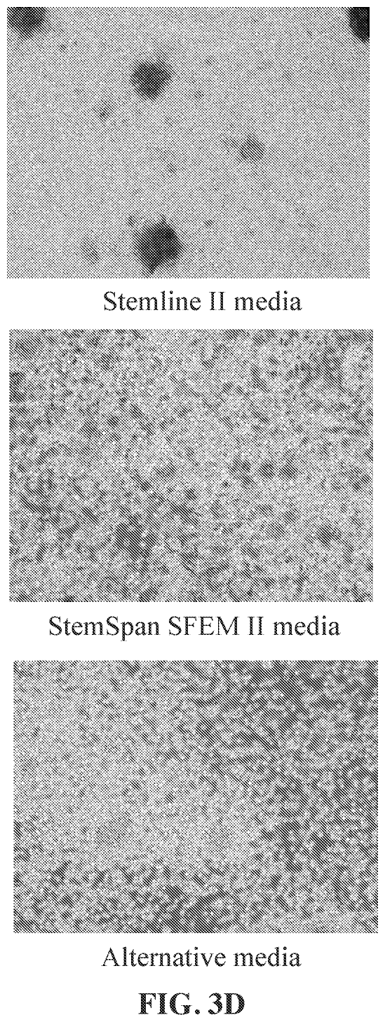

High-purity iNKs were observed, with 97.6%, 72.6%, and 63.1% of all cells being quadruple-positive for NK markers CD43/CD45/CD56/LFA1 (FIG. 3A) when cultured with Stemline II, StemSpan SFEM II, or an alternative media, respectively (FIG. 3B). Highest yields of iNKs were observed with Stemline II media, at 316-fold expansion of iNKs at day 40 relative to iPSCs seeded at day 0, and lower-fold expansions were observed with StemSpan SFEM II or an alternative media, at 20.9- and 7.6-fold expansions, respectively (FIG. 3C). Representative brightfield microscope images are shown of the cells at day 39 of iNK differentiation (FIG. 3D).

This method resulted in a high yield of hematopoietic progenitors (HPs) after 9-18 days of differentiation based on CD34+/CD43+/CD45+ marker expression, and high purity and yield of NKs after 40 days of differentiation based on CD43+/CD45+/CD56+/LFA1+ marker expression: >90% of all cells at day 40 are NK cells.

### Analysis of Differentiation Protocol

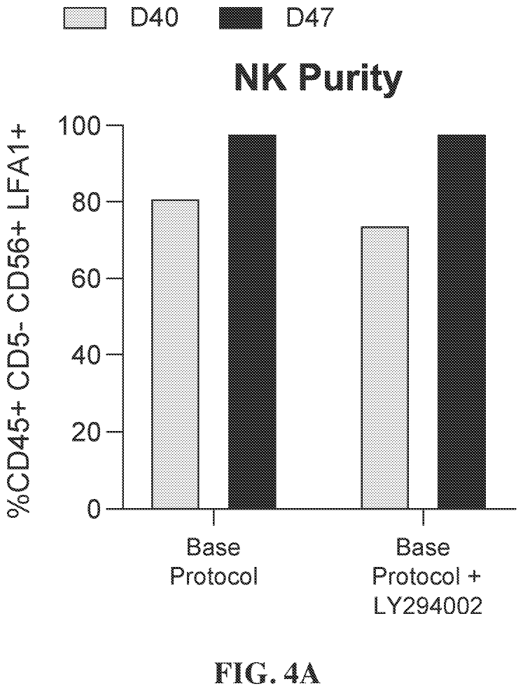

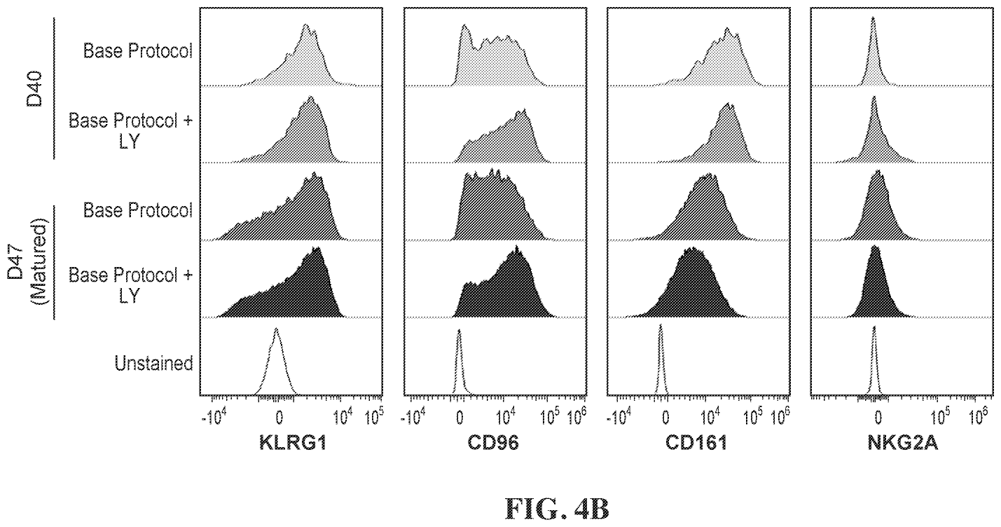

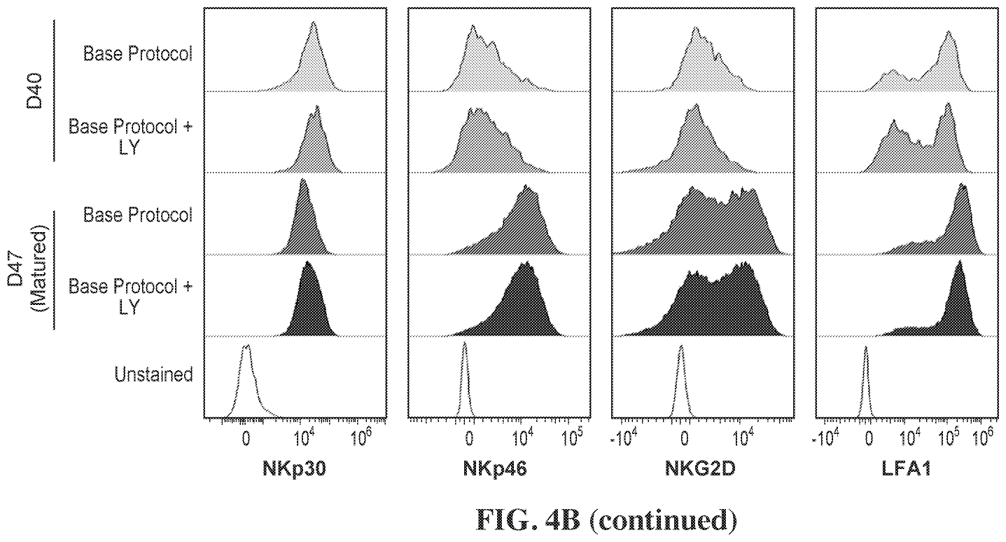

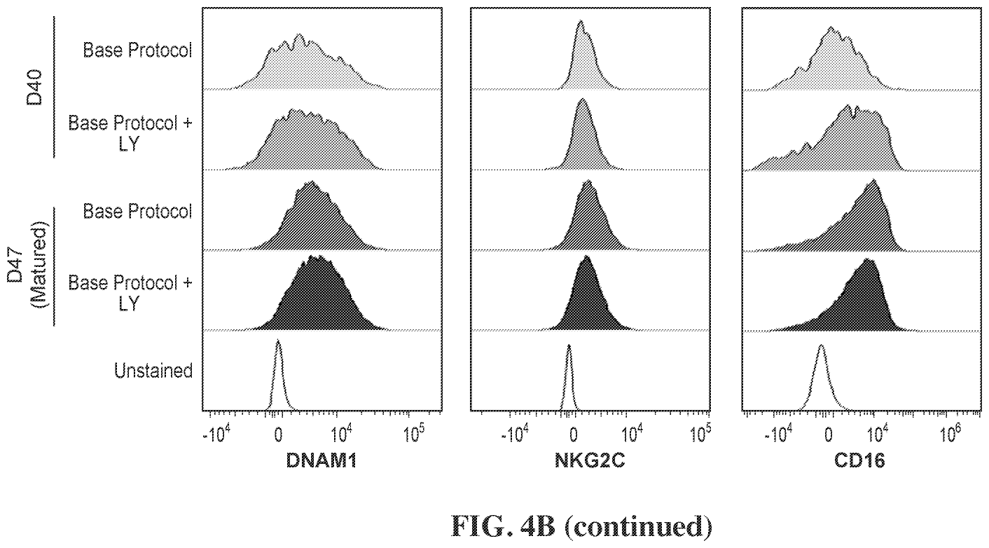

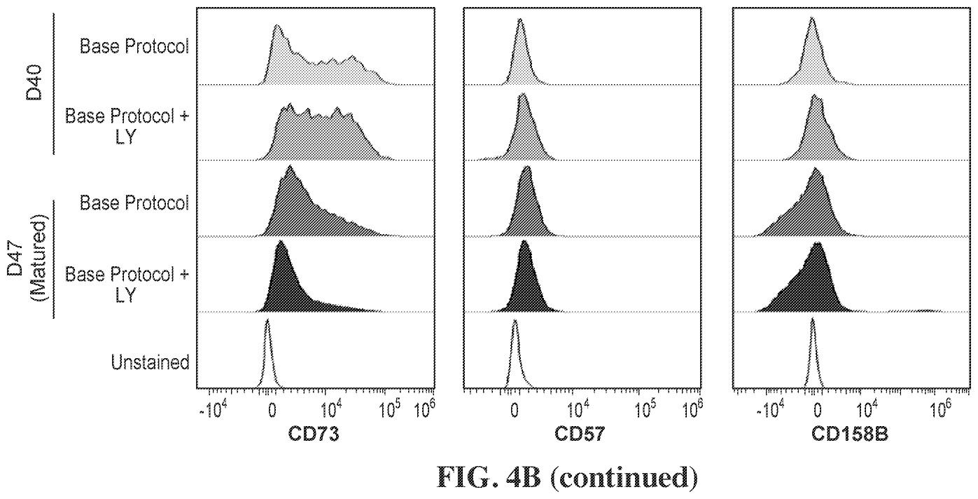

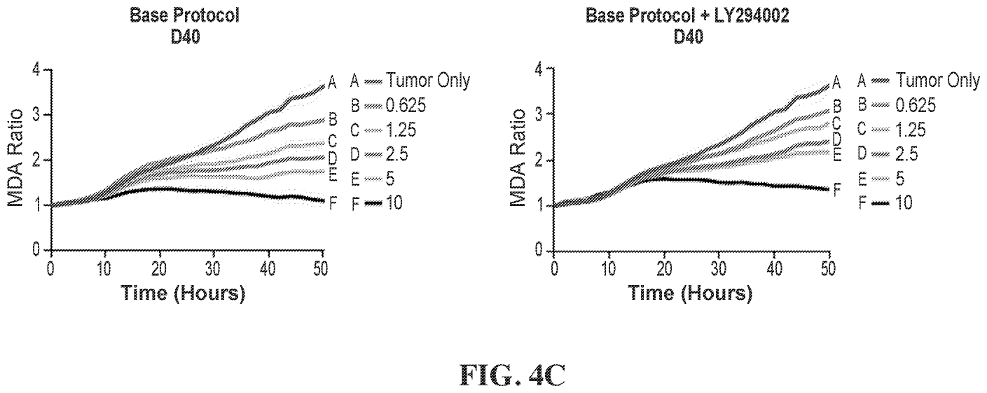

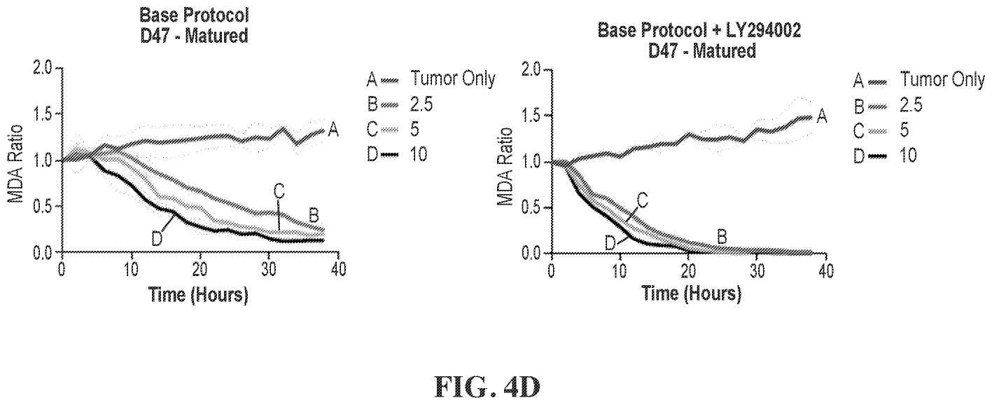

Cells were incubated for 40 days using the following protocol: Days 0-2, BMP4/FGF/VEGF (10/10/50)+RI; Days 3-14, BMP4/FGF/VEGF/SCF/LDL/TPO/+/−LY294002; Days 15-40, SCF/FLT3/IL15/IL7/IL12/UM729/SR1. The cells were further incubated with the aforementioned base protocol+LY294002. where indicated. Immunophenotyping of day 40 harvested iNKs showed purity of ˜80% as indicated by CD45+CD5− CD56+ LFA1+ cells. Day 40 cells were then incubated for one week in a maturation medium containing (IL-2, IL-7, IL-12, IL-15, IL-18 and activation beads, anti-CD2/NKp46) (FIG. 4A). Immunophenotyping showed increased purity of NK cells post maturation (˜98%) as well as increases in activation markers NKp46, NKG2D, LFA1, CD16 and decreases in inhibitory markers CD161 and CD73 (FIG. 4B).

D40 iNKs (FIG. 4C) or D47 matured iNKs (FIG. 4D) were incubated with breast adenocarcinoma MDA-MB231 cells expressing a nuclear fluorescent protein at different T:E ratios in the presence of IL-2, IL-7, IL-15. Cell mixtures were placed into an IncuCyte fluorescent microscope and imaged every 2 hours. MDA cells were quantified over time via fluorescent marker and graphed as the ratio of MDA cells compared to time 0 (time 0 is equal to one). iNK cells reduced MDA growth in a dose-responsive manner and maturation as well as LY294002 addition seemed to increase innate targeting of MDA cells.

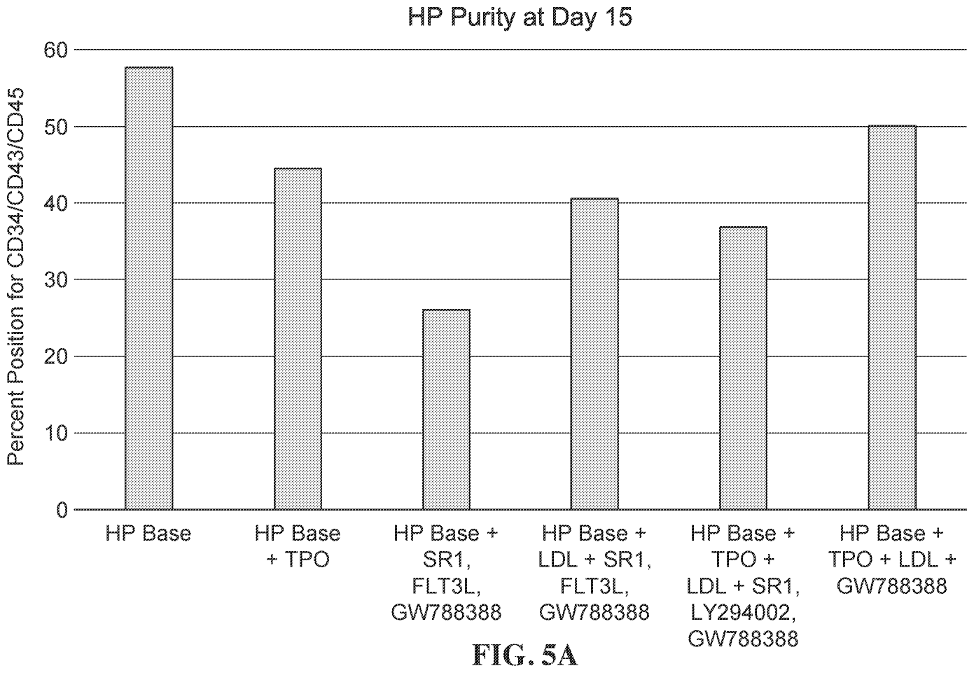

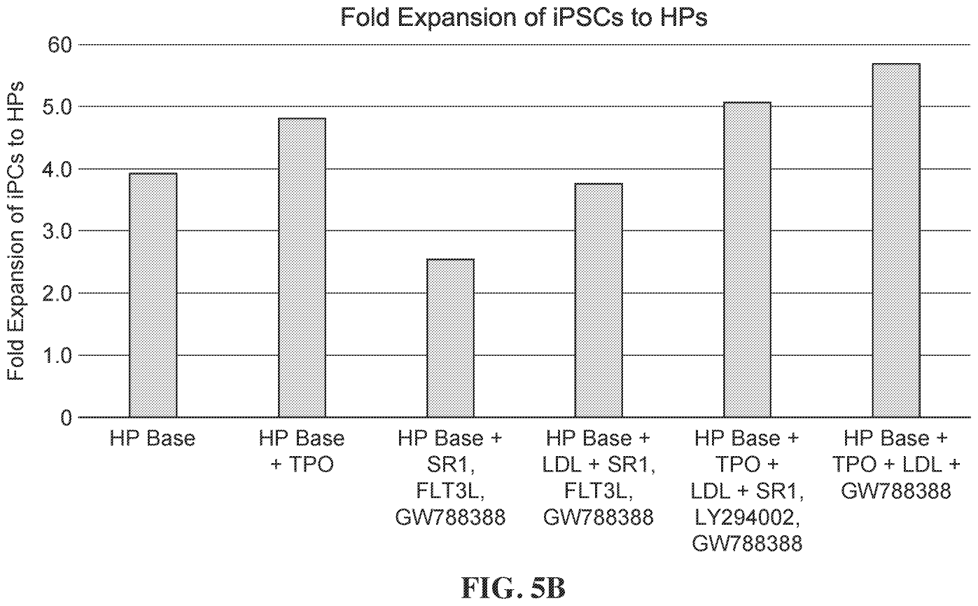

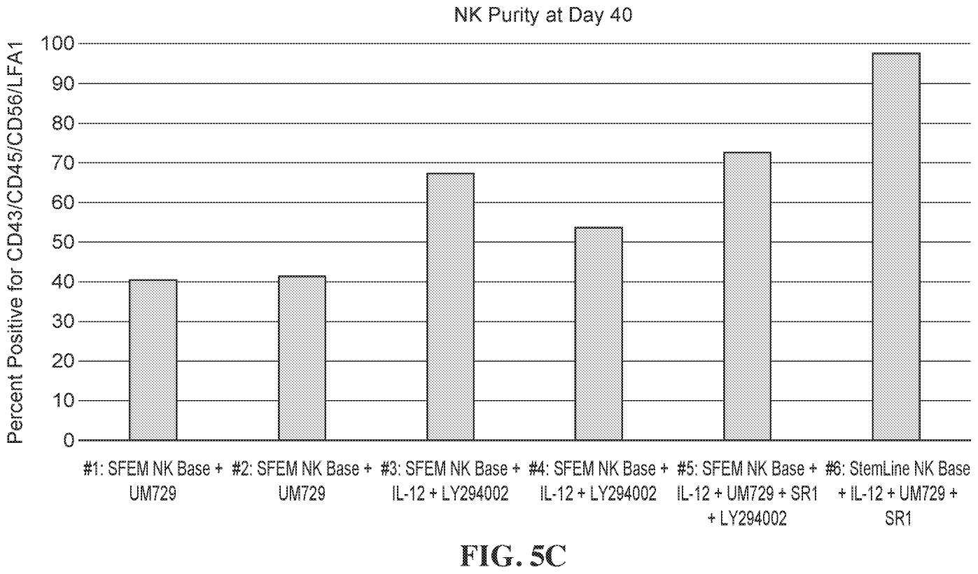

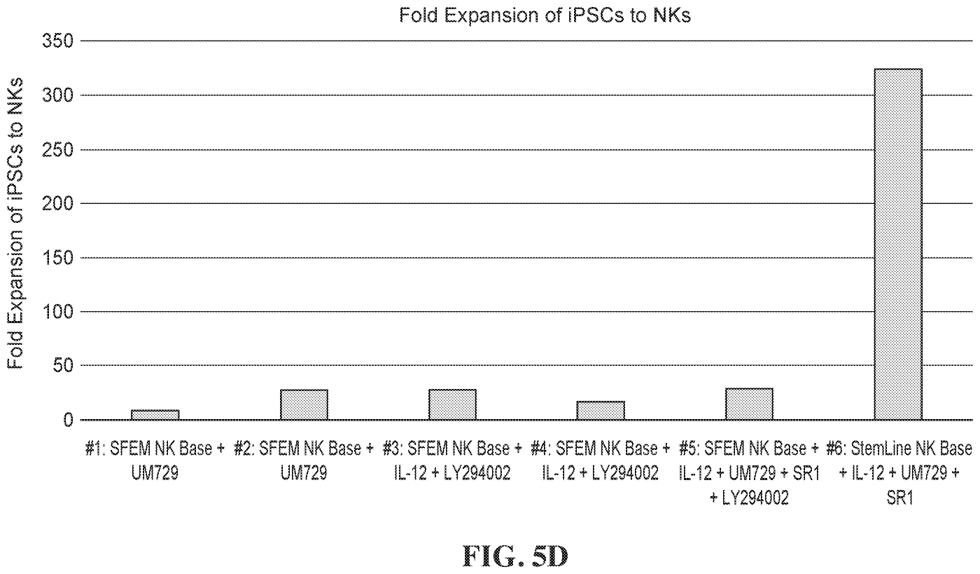

The iNK differentiation protocol was created over time by modifying variables over four experiments. D15 HPs (FIG. 5A) and (FIG. 5B) show results from iPSCs cultured in SFEM II or Stemline media containing various combinations of the following: Y-27632, BMP4, FGF2, VEGF, SCF, LDL, TPO, SR1, UM171, FLT3L, GW and LY. HP Base media included StemSpan SFEM II media supplemented with BMP4, FGF2, VEGF, and Y27632. HP purity was observed in ranges of 10-58% of all cells being triple-positive for CD34/CD43/CD45 (FIG. 5A). Fold expansion of iPSCs to HPs was observed in ranges of 0.4-11.7 on day 15, relative to iPSCs seeded at day 0 (FIG. 5B). Additives included in FIG. 5A and FIG. 5B: SR1, FLT3L, LY294002, and GW788388. The NK Base media includes SCF, IL-7, FLT3L, and IL-15 D40. iNKS (FIG. 5C) and (FIG. 5D) show results from HPs cultured in SFEM II or StemLine media containing various combinations of the following: SCF, IL-7, IL-15, FLT3L, UM729, IL-12, IL-18, SB431542 and SR1. NK Base media included StemSpan SFEM II (#1-5) or StemLine II (#6) media supplemented with SCF, IL-7, FLT3L, and IL-15. For experiments in FIG. 5C and FIG. 5D, HP media used included HP Base with the following additions for experiments #1-6, respectively: no additions (#1); LY294002 and TPO (#2); TPO and GW788388 (#3); LY294002 and GW788388 (#4); LY294002, TPO, and GW788388 (#5); LY294002, LDL, and TPO (#6); LDL and TPO. On day 40, iNKs, defined as CD43+/CD45+/CD56+, showed NK purity ranging from 33.4-97.6% (FIG. 5C). Day 40 iNKs showed fold expansions of iPSCs to iNKs ranging from 8.62 to 323.9 (FIG. 5D).

### Example 2: Development of NK Cell Differentiation Method to Increase the Yield Ratio of HP/iPSC and NK/iPSC

The purpose of this study was to evaluate different sources of BMP4, to determine if the use of heat stable FGF or if the addition of LY at the HP differentiation stage increases the yield of HP and iNK cells from iPSCs.

### Formation of Embryoid Bodies (EBs)

EBs were induced to form from iPSC cells as described in FIG. 1, with the following exceptions: using a base media without BMP4 or FGF (See Table 1), Peprotech BMP4 and Peprotech FGF was added for Condition 1P (See Table 2), Invitrogen BMP4 and Peprotech FGF was added for Condition 1I (See Table 3), BioLegend BMP4 and Peprotech FGF was added for Condition 1B (See Table 4), Peprotech BMP4 and Peprotech FGF was added for Condition 2P (See Table 5), Peprotech BMP4 and Gibco Heat Stable (HS) FGF was added for Condition 2G (See Table 6), and Peprotech BMP4 and Peprotech FGF was added for Condition 3 (See Table 7).

TABLE 1
Base Seeding Media
Base Seed Media without BMP4 and FGF

| Component | Amount | [Stock] | [Final] |
|---|---|---|---|
| STEMdiff APEL 2 | 61 ml | NA | NA |
| BMP4 | See below | 10 ng/uL | 10 ng/mL |
| FGF2 | See below | 50 ng/uL | 10 ng/mL |
| VEGF-165 | 61 ul | 50 ng/uL | 50 ng/mL |
| Y-27632 | 61 ul | 10 mM | 10 uM |

TABLE 2
Media for Condition 1P
Condition 1P: Premade Seed media + Peprotech BMP4 & Peprotech FGF

| Component | Amount | [Stock] | [Final] |
|---|---|---|---|
| STEMdiff APEL 2 | 6.5 ml | NA | NA |
| BMP4 | 6.5 ul | 10 ng/uL | 10 ng/mL |
| FGF2 | See below | 50 ng/uL | 10 ng/mL |
| VEGF-165 | NA | 50 ng/uL | 50 ng/mL |
| Y-27632 | NA | 10 mM | 10 uM |

TABLE 3
Media for Condition 1I
Condition 1I: Premade Seed media + Invitrogen BMP4 & Peprotech FGF

| Component | Amount | [Stock] | [Final] |
|---|---|---|---|
| STEMdiff APEL 2 | 61 ml | NA | NA |
| BMP4 | See below | 10 ng/uL | 10 ng/mL |
| FGF2 | See below | 50 ng/uL | 10 ng/mL |
| VEGF-165 | 61 ul | 50 ng/uL | 50 ng/mL |
| Y-27632 | 61 ul | 10 mM | 10 uM |

TABLE 4
Media for Condition 1B
Condition 1B: Premade Seed media + BioLegend BMP4 & Peprotech FGF

| Component | Amount | [Stock] | [Final] |
|---|---|---|---|
| STEMdiff APEL 2 | 6.5 ml | NA | NA |
| BMP4 | 6.5 ul | 10 ng/uL | 10 ng/mL |
| FGF2 | See below | 50 ng/uL | 10 ng/mL |
| VEGF-165 | NA | 50 ng/uL | 50 ng/mL |
| Y-27632 | NA | 10 mM | 10 uM |

TABLE 5
Media for Condition 2P
Condition 2P: Premade Seed media + Peprotech BMP4 & Peprotech FGF

| Component | Amount | [Stock] | [Final] |
|---|---|---|---|
| STEMdiff APEL 2 | 6.5 ml | NA | NA |
| BMP4 | See above | 10 ng/uL | 10 ng/mL |
| FGF2 | 1.3 ul | 50 ng/uL | 10 ng/mL |
| VEGF-165 | NA | 50 ng/uL | 50 ng/mL |
| Y-27632 | NA | 10 mM | 10 uM |

TABLE 6
Media for Condition 2G
Condition 2G: Premade Seed media + Peprotech BMP4 & Gibco HS FGF

| Component | Amount | [Stock] | [Final] |
|---|---|---|---|
| STEMdiff APEL 2 | 6.5 ml | NA | NA |
| BMP4 | See above | 10 ng/uL | 10 ng/mL |
| FGF2 | 1.3 ul | 50 ng/uL | 10 ng/mL |
| VEGF-165 | NA | 50 ng/uL | 50 ng/mL |
| Y-27632 | NA | 10 mM | 10 uM |

TABLE 7
Media for Condition 3
Condition 3: Premade Seed media + Peprotech BMP4 & Peprotech FGF

| Component | Amount | [Stock] | [Final] |
|---|---|---|---|
| STEMdiff APEL 2 | 26 ml | NA | NA |
| BMP4 | 2.6 ul | 10 ng/uL | 10 ng/mL |
| FGF2 | 5.2 ul | 50 ng/uL | 10 ng/mL |
| VEGF-165 | NA | 50 ng/uL | 50 ng/mL |
| Y-27632 | NA | 10 mM | 10 uM |

The results of this experiment determined that EBs formed under all conditions without any visual differences between treatments.

### Formation of Hematopoietic Progenitors (HPs)

HPs were induced to form from iPSC-derived EBs as described in FIG. 1, with the following exceptions: using a HP Differentiation media without BMP4 or FGF (See Table 8), Peprotech BMP4 and Peprotech FGF was added for Condition 1P, Invitrogen BMP4 and Peprotech FGF was added for Condition 1I, BioLegend BMP4 and Peprotech FGF was added for Condition 1B, Peprotech BMP4 and Peprotech FGF was added for Condition 2P, Peprotech BMP4 and Gibco Heat Stable (HS) FGF was added for Condition 2G, Peprotech BMP4 and Peprotech FGF was added for Condition 3, and Peprotech BMP4, Peprotech FGF and LY was added for Condition 3+LY.

The cells were visually inspected by brightfield microscopy day 13 and the cells for Condition 3 and Condition 3+LY appeared sufficient for harvest (data not shown). To determine the percentage of HPs generated of the total cells present at day 13 of differentiation, flow cytometry analysis was performed, gating cells to quantify percentage triple-positive for the HP markers CD34/CD43/CD45 (See Table 9). All conditions were then harvested at Day 15 of differentiation and flow cytometry analysis was performed, gating cells to quantify percentage triple-positive for the HP markers CD34/CD43/CD45 (See Table 10). The comparison of harvesting at Day 13 vs Day 15 is shown in Table 11 and Table 12.

TABLE 8
HP Differentiation Media

| Component | Amount | [Stock] | [Final] |
|---|---|---|---|
| SFEM II | 240 ml | | NA |
| BMP4 | Add Later | 10 ng/ul | 10 ng/ml |
| FGF2 | Add Later | 50 ng/ul | 50 ng/ml |
| VEGF-165 | 240 ul | 50 ng/ul | 50 ng/ml |
| SCF | 240 ul | 50 ng/ul | 50 ng/ml |
| TPO | 240 ul | 25 ng/ul | 25 ng/ml |
| LDL | 480 ul | 5 ug/ul | 10 ug/ml |

TABLE 9
Percentage of Triple-Positive Cells at Day 13

| Sample ID (Day 13) | CD34/43/45 (%) | HPs per iPSC |
|---|---|---|
| Pepro (BMP4 + FGF) | 38 | 3.5 |
| Pepro (BMP4 + FGF) + LY | 55 | 5.5 |

TABLE 10
Percentage and Total Cell Counts of Triple-Positive Cells at Day 15

| | | | | Total | Total | Absolute | |
|---|---|---|---|---|---|---|---|
| Sample ID | CD45+ | CD34+CD43+ | CD34/43/45 | Count Per | Cell | HP | |
| (Day 15) | (%) | (%) | (%) | Plate | Count | Count | HP/iPSC |
| BioL BMP4 | 99.2 | 18 | 18 | 1.11 × 10\hat{ }6 | 2.22 × 10\hat{ }6 | 3.99 × 10\hat{ }5 | 0.83 |
| Invitro BMP4 | 99.2 | 17 | 17 | 8.40 × 10\hat{ }5 | 1.68 × 10\hat{ }6 | 2.80 × 10\hat{ }5 | 0.58 |
| Pepro BMP4 | 97.4 | 47 | 46 | 9.60 × 10\hat{ }5 | 1.92 × 10\hat{ }6 | 8.79 × 10\hat{ }5 | 1.83 |
| HS FGF | 97.9 | 46 | 45 | 1.05 × 10\hat{ }6 | 2.10 × 10\hat{ }6 | 9.54 × 10\hat{ }5 | 1.99 |
| Pepro FGF | 96.2 | 42 | 41 | 1.33 × 10\hat{ }6 | 2.66 × 10\hat{ }6 | 1.08 × 10\hat{ }6 | 2.26 |
| Pepro | 96.7 | 54 | 52 | 1.35 × 10\hat{ }6 | 5.40 × 10\hat{ }6 | 2.80 × 10\hat{ }6 | 2.92 |
| (BMP4 + FGF) | | | | | | | |
| Pepro | 94.1 | 56 | 53 | 1.02 × 10\hat{ }6 | 4.08 × 10\hat{ }6 | 2.15 × 10\hat{ }6 | 2.24 |
| (BMP4 + | | | | | | | |
| FGF) + LY | | | | | | | |

TABLE 11
Comparison of Yield Ratios from Day 13 vs Day 15

| | Day 13 | | Day 15 | |
|---|---|---|---|---|
| | CD34/43/45 | HP/iPSC | CD34/43/45 | HP/iPSC |
| BioL BMP4 | | | 23% | 1.1 |
| Invitro BMP4 | | | 23% | 0.8 |
| HS FGF | | | 54% | 2.4 |
| Pepro both | 55% | 5.5 | 47% | 2.3 |
| Pepro both + LY | 35% | 3.5 | 54% | 2.3 |

TABLE 12
Comparison of Yield Ratios from Day 13 vs Day 15

| | Day 13 | Day 15 | From Day 13-15 | |
|---|---|---|---|---|
| | Estimated Cell | Cell | Increase in | Change in |
| | Count/plate | count/plate | total count | HP/iPSC |
| LY | 8.4 × 10\hat{ }5 | 1.02 × 10\hat{ }6 | 18% | 34% decrease |
| No LY | 1.32 × 10\hat{ }6 | 1.35 × 10\hat{ }6 | 2% | 56% decrease |

The results of this experiment determined that HPs formed under all conditions but harvesting the HPs at day 13 resulted in a higher yield ratio of HP/iPSC.

Formation of iNKs

NKs were induced to form from HPs as described in FIG. 1, with the following exceptions: using a NK Differentiation media without BMP4 or FGF (See Table 13), Peprotech BMP4 and Peprotech FGF was added for Condition 1P, Invitrogen BMP4 and Peprotech FGF was added for Condition 1I, BioLegend BMP4 and Peprotech FGF was added for Condition 1B, Peprotech BMP4 and Peprotech FGF was added for Condition 2P, Peprotech BMP4 and Gibco Heat Stable (HS) FGF was added for Condition 2G, Peprotech BMP4 and Peprotech FGF was added for Condition 3, and Peprotech BMP4, Peprotech FGF and LY was added for Condition 3+LY.

TABLE 13
NK Differentiation Media

| Component | Amount | [Stock] | [Final] |
|---|---|---|---|
| SFEM II | 225 ml | | NA |
| SCF | 225 ul | 20 ng/ul | 20 ng/ml |
| IL-7 | 225 ul | 20 ng/ul | 20 ng/ml |
| IL-12 | 225 ul | 20 ng/ul | 20 ng/ml |
| IL-15 | 225 ul | 10 ng/ul | 10 ng/ml |
| FLT3L | 225 ul | 10 ng/ul | 10 ng/ml |
| SR1 | 225 ul | 10 mM | 1 uM |
| UM729 | 22.5 ul | 10 mM | 1 uM |

To determine the percentage of NKs generated of the total cells present at day 40 of differentiation, flow cytometry analysis was performed, gating cells to quantify percentage of NK cells (Markers CD34+/CD43+/CD45+/LFA1+/CD56+) (See Table 14).

TABLE 14
Percentage and Total Cell Counts of NK Cells at Day 40

| Sample ID | Monocytes | NK Cells | Cell | Absolute | |
|---|---|---|---|---|---|
| (Day 40) | (%) | (%) | Ct | NKs | NK/iPSC |
| Condition 1B, | 66.2 | 33.3 | 2.88 × 10\hat{ }6 | 9.59 × 10\hat{ }5 | 4 |
| BioL BMP4 | | | | | |
| Condition 1I, | 84.7 | 15.2 | 1.3 × 10\hat{ }6 | 1.98 × 10\hat{ }5 | 0.82 |
| Invitro BMP4 | | | | | |
| Condition 1P, | 41.2 | 58 | 6.5 × 10\hat{ }6 | 3.77 × 10\hat{ }6 | 15.71 |
| Pepro BMP4 | | | | | |
| Condition 2G, | 94 | 5.25 | 8 × 10\hat{ }5 | 4.2 × 10\hat{ }4 | 0.18 |
| HS FGF | | | | | |
| Condition 2P, | 51.6 | 47.6 | 6.24 × 10\hat{ }6 | 2.97 × 10\hat{ }6 | 12.38 |
| Pepro FGF | | | | | |
| Condition 3 + LY, | 47 | 52.2 | 1.7 × 10\hat{ }7 | 8.87 × 10\hat{ }6 | 36.98 |
| Pepro (BMP4 + | | | | | |
| FGF) + LY | | | | | |
| Condition 3, | 47.6 | 51.5 | 1.18 × 10\hat{ }7 | 6.08 × 10\hat{ }6 | 25.32 |
| Pepro (BMP4 + FGF) | | | | | |

The results of this experiment determined that NKs formed under all conditions but the addition of a ROCK inhibitor resulted in the highest NK/iPSC yield ratio.

1. A method for generating a population of CD34+/CD43+/CD45+ cells, comprising contacting a population of stem cells with a differentiation media comprising a bone morphogenetic protein (BMP) pathway activator, a fibroblast growth factor (FGF), and a vascular endothelial growth factor (VEGF), for a period of time sufficient to generate the population of CD34+/CD43+/CD45+ cells from the population of stem cells.

2. A method for differentiation a population of stem cells into a population of hematopoietic progenitors, comprising contacting the population of stem cells with a differentiation media comprising a bone morphogenetic protein (BMP) pathway activator, a fibroblast growth factor (FGF), and a vascular endothelial growth factor (VEGF), for a period of time sufficient to differentiate the population of stem cells into the population of hematopoietic progenitors.

3. The method of claim 2, wherein the population of hematopoietic progenitors comprises CD34+/CD43+/CD45+ cells.

4. The method of any one of claims 1-3, wherein the BMP pathway activator is BMP4.

5. The method of any one of claims 1-4, wherein the FGF is FGF2.

6. The method of any one of claims 1-5, wherein the VEGF is VEGF-165.

7. The method of any one of claims 1-6, wherein the differentiation media comprises Rho-associated coiled coil forming protein serine/threonine kinase (ROCK) inhibitor.

8. The method of claim 7, wherein the ROCK inhibitor is Y27632.

9. The method of any one of claims 1-8, wherein the differentiation media comprises stem cell factor (SCF).

10. The method of any one of claims 1-9, wherein the differentiation media comprises thrombopoietin (TPO).

11. The method of any one of claims 1-10, wherein the differentiation media comprises a low-density lipoprotein (LDL).

12. The method of any one of claims 1-11, wherein the differentiation media comprises a phosphoinositide 3-kinase (PI3K) inhibitor.

13. The method of claim 12, wherein the PI3K inhibitor is LY294002.

14. The method of any one of claims 1-13, wherein the differentiation media comprises a pyrimido-[4,5-b]-indole derivative.

15. The method of claim 14, wherein the pyrimido-[4,5-b]-indole derivative is UM729.

16. The method of any one of claims 1-15, wherein the differentiation media comprises an aryl hydrocarbon receptor (AhR) antagonist.

17. The method of claim 16, wherein the AhR antagonist is StemRegenin 1 (SR1).

18. The method of any one of claims 1-17, wherein the differentiation media comprises the BMP pathway activator, the FGF, the VEGF, and the ROCK inhibitor.

19. The method of any one of claims 1-17, wherein the differentiation media comprises the BMP pathway activator, the FGF, the VEGF, SCF, TPO, the LDL and the inhibitor of PI3K.

20. The method of any one of claims 1-17, wherein the differentiation media comprises the BMP pathway activator, the FGF, the VEGF, SCF, TPO, the LDL, the PI3K inhibitor, the pyrimido-[4,5-b]-indole derivative, and the AhR antagonist.

21. The method of any one of claims 1-6, comprising contacting the population of stem cells with the differentiation media for 1-5 days, wherein the differentiation media comprises the BMP pathway activator, the FGF, the VEGF, and optionally the ROCK inhibitor.

22. The method of any one of claims 1-13, comprising (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, and the PI3K inhibitor.

23. The method of any one of claims 1-17, comprising (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, the PI3K inhibitor, the pyrimido-[4,5-b]-indole derivative, and the AhR antagonist.

24. The method of any one of claims 1-23, wherein the differentiation media comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

25. The method of any one of claims 1-24, wherein the stem cells are induced pluripotent stem cells (iPSCs).

26. The method of any one of claims 1-24, wherein the stem cells are human embryonic stem cells (hESCs).

27. A method for generating a population of CD43+/CD45+/CD56+/LFA1+ cells, comprising contacting a population of CD34+/CD43+/CD45+ cells with a media comprising SCF, interleukin-7 (IL-7), IL-12, IL-15, FMS-like tyrosine kinase 3 ligand (FLT3L), a pyrimido-[4,5-b]-indole derivative, and an AhR inhibitor, for a period of time sufficient to generate the population of CD43+/CD45+/CD56+/LFA1+ cells from the population of CD34+/CD43+/CD45+.

28. A method of differentiating a population of hematopoietic progenitors and/or common lymphoid progenitors into a population of Natural Killer (NK) cells, comprising contacting the population of hematopoietic progenitors with a differentiation media comprising SCF, IL-7, IL-12, IL-15, FLT3L, a pyrimido-[4,5-b]-indole derivative, and an AhR inhibitor, for a period of time sufficient to differentiate the population of hematopoietic progenitors into the population of NK cells.

29. The method of claim 27 or 28, wherein the pyrimido-[4,5-b]-indole derivative is UM729.

30. The method of any one of claims 27-29, wherein the AhR inhibitor is SR1.

31. The method of any one of claims 28-30, wherein the media comprises 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

32. The method of any one of claims 27-31, wherein the period of time is 11-25 days.

33. The method of any one of claims 27-32, further comprising maturing the population of NK cells with a maturation media comprising (i) IL-12, IL-15 and IL-18, or (ii) IL-12, IL-2 and IL-18.

34. The method of any one of claims 1-33, wherein the differentiation media and/or maturation media is serum free.

35. The method of any one of claims 1-34, wherein the method is xenogenic-free.

36. A method of generating a population of NK cells, comprising:
(a) obtaining a population of stem cells;
(b) contacting the population of stem cells with a first media comprising a BMP pathway activator, an FGF, a VEGF, and optionally an inhibitor of ROCK, for a period of time sufficient to generate embryoid bodies;
(c) contacting the embryoid bodies with a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor, for a period of time sufficient to generate a population of hematopoietic progenitors;
(d) contacting the population of hematopoietic progenitors with a second differentiation media comprising SCF, IL-7, IL-12, IL-15, FLT3L, a pyrimido-[4,5-b]-indole derivative, and an AhR inhibitor, for a period of time sufficient to generate the population of NK cells.

37. The method of claim 36, wherein the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

38. The method of claim 36 or 37, wherein each media of steps (b)-(d) is serum free.

39. The method of any one of claims 36-38, wherein the method is xenogenic-free.

40. The method of any one of claims 36-39, where the first media, the first differentiation media, and the second differentiation media each comprise the same base media.

41. The method of any one of claims 36-39, where the first media, the first differentiation media, and the second differentiation media each comprise different base media.

42. The method of any one of claims 36-39, where the first differentiation media and the second differentiation media each comprise the same base media, and the first media comprises a base media different from the first and second differentiation media.

43. The method of any one of claims 36-39, wherein the first differentiation media and the second differentiation media each comprise a base media comprising Iscove's modified dulbecco's medium, bovine serum albumin, recombinant human insulin, human transferrin, and 2-mercaptoethanol.

44. The method of any one of claims 36-43, wherein the period of time of step (b) is 1-5 days, the period of time of step (c) is 3-15 days, and the period of time of step (d) is 11-25 days.

45. The method of any one of claims 36-44, wherein steps (a)-(d) occur within 35-45 days.

46. The method of any one of claims 36-45, comprising (e) expanding the population of NK cells with a maturation media comprising (i) IL-12, IL-15 and IL-18, or (ii) IL-12, IL-2 and IL-18.

47. The method of any one of claims 36-46, wherein the stem cells are induced pluripotent stem cells (iPSCs) or human embryonic stem cells (hESCs).

48. The method of any one of claims 36-47, wherein the population of hematopoietic progenitors comprises about 30% to about 50% CD34+/CD43+/CD45+ cells.

49. The method of any one of claims 36-48 wherein the population of NK cells comprises about 60% to about 100% CD43+/CD45+/CD56+/LFA1+ cells.

50. The method of any one of claims 36-49, comprising expanding the population of NK cells, wherein the population of NK cells expands about 10 to about 350 fold.

51. The method of any one of claims 1-26 and 36-50, wherein the population of stem cells is genetically engineered or edited.

52. The method of any one of claims 27-51, wherein the population of NK cells is genetically engineered or edited.

53. A population of cells comprising hematopoietic progenitors produced by the method of any one of claims 1-26.

54. The population of cells of claim 53, wherein the hematopoietic progenitors are CD34+/CD43+/CD45+.

55. The population of cells of claim 53 or 54, comprising 30-50% hematopoietic progenitors.

56. A population of cells comprising NK cells produced by the method of any one of claims 27-52.

57. The population of cells of claim 56, wherein the NK cells are CD43+/CD45+/CD56+/LFA1+.

58. The population of cells of claim 56 or 57, comprising 60-100% NK cells.

59. A hematopoietic progenitor differentiation media comprising a serum-free base media, a BMP pathway activator, an FGF, a VEGF, SCF, TPO, LDL, and a PI3K inhibitor.

60. The hematopoietic progenitor differentiation media of claim 59, wherein the BMP pathway activator is BMP4, the FGF is FGF2, the VEGF is VEGF-165, and the PI3K inhibitor is LY294002.

61. The hematopoietic progenitor differentiation media of claim 59 or 60, comprising 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

62. An NK cell differentiation media comprising a serum-free base media, SCF, IL-7, IL-12, 11-15, FLT3L, a pyrimido-[4,5-b]-indole derivative, and an AhR inhibitor.

63. The NK cell differentiation media of claim 62, wherein the pyrimido-[4,5-b]-indole derivative is UM729 and the AhR inhibitor is SR1.

64. The NK cell differentiation media of claim 62 or 63, comprising 1-100 ng/mL SCF, 1-50 ng/mL IL-7, 1-100 ng/mL IL-12, 1-100 ng/mL IL-15, 1-100 ng/mL FLT3L, 0.1-10 uM pyrimido-[4,5-b]-indole derivative, 0.1-10 uM AhR antagonist, and any combination thereof.

65. A kit comprising the hematopoietic progenitor differentiation media of any one of claims 59-61 and instructions for contacting a population of stem cells with the hematopoietic progenitor differentiation media for a period of time sufficient to generate a population of cells comprising hematopoietic progenitors.

66. The kit of claim 65, wherein the period of time is 1-15 days.

67. A kit comprising the NK cell differentiation media of any one of claims 62-64 and instructions for contacting a population of hematopoietic progenitors with the NK cell differentiation media for a period of time sufficient to generate a population of cells comprising NK cells.

68. The kit of claim 67, wherein the period of time is 11-25 days.

69. A kit comprising the hematopoietic progenitor differentiation media of any one of claims 59-61 and the NK cell differentiation media of any one of claims 62-64, and instructions for contacting a population of stem cells with the hematopoietic progenitor differentiation media for a first period of time sufficient to generate a population of cells comprising hematopoietic progenitors, and contacting the population of cells comprising hematopoietic progenitors with the NK cell differentiation media for a second period of time sufficient to generate a population of cells comprising NK cells.

70. The kit of claim 69, wherein the first period of time is 1-15 days, and the second period of time is 11-25 days.

71. The kit of any one of claims 67-70, further comprising a maturation media comprising a base media and (i) IL-12, IL-15 and IL-18, or (ii) IL-12, IL-2 and IL-18, and instructions for contacting the population of cells comprising NK cells for a period of time sufficient to mature the NK cells.

72. A composition to increase the yield ratio of hematopoietic progenitors from a population of stem cells, the composition comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor.

73. The composition of claim 72, wherein the hematopoietic progenitors comprise CD34+/CD43+/CD45+ cells.

74. The composition of any one of claims 72-73, wherein the BMP pathway activator is BMP4.

75. The composition of any one of claims 72-74, wherein the FGF is FGF2.

76. The composition of any one of claims 72-75, wherein the VEGF is VEGF-165.

77. The composition of claim 76, wherein the ROCK inhibitor is Y27632.

78. The composition of any one of claims 72-77, wherein the composition further comprises stem cell factor (SCF).

79. The composition of any one of claims 72-78, wherein the composition further comprises thrombopoietin (TPO).

80. The composition of any one of claims 72-79, wherein the composition further comprises a low-density lipoprotein (LDL).

81. The composition of any one of claims 72-80, wherein the composition further comprises a phosphoinositide 3-kinase (PI3K) inhibitor.

82. The composition of claim 81, wherein the PI3K inhibitor is LY294002.

83. The method of any one of claims 72-82, wherein the composition further comprises a pyrimido-[4,5-b]-indole derivative.

84. The composition of claim 83, wherein the pyrimido-[4,5-b]-indole derivative is UM729.

85. The composition of any one of claims 72-84, wherein the composition further comprises an aryl hydrocarbon receptor (AhR) antagonist.

86. The composition of claim 85, wherein the AhR antagonist is StemRegenin 1 (SR1).

87. The composition of any one of claims 72-86, wherein the composition comprises the BMP pathway activator, the FGF, the VEGF, and the ROCK inhibitor.

88. The composition of any one of claims 72-87, wherein the composition comprises the BMP pathway activator, the FGF, the VEGF, SCF, TPO, the LDL and the inhibitor of PI3K.

89. The composition of any one of claims 72-88, wherein the composition comprises the BMP pathway activator, the FGF, the VEGF, SCF, TPO, the LDL, the PI3K inhibitor, the pyrimido-[4,5-b]-indole derivative, and the AhR antagonist.

90. The composition of any one of claims 72-89, wherein the composition comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

91. The composition of any one of claims 72-90, wherein the population of stem cells are induced pluripotent stem cells (iPSCs).

92. The composition of any one of claims 72-91, wherein the population of stem cells are human embryonic stem cells (hESCs).

93. The composition of any one of claims 72-92, wherein the yield ratio of hematopoietic progenitor cells (HP) to stem cell (StC) (HP/StC) is about 2:1 to about 10:1.

94. The composition of claim 93, wherein the yield ratio of hematopoietic progenitor cells (HP) to stem cell (StC) (HP/StC) is about 5:1.

95. A method to increase the yield ratio of hematopoietic progenitor cells from a population of stem cells, comprising contacting the population of stem cells with a differentiation media comprising a bone morphogenetic protein (BMP) pathway activator, a fibroblast growth factor (FGF), a vascular endothelial growth factor (VEGF), and a Rho-associated coiled coil forming protein serine/threonine kinase (ROCK) inhibitor for a period of time sufficient to differentiate the population of stem cells into the hematopoietic progenitors.

96. The method of claim 95, wherein the hematopoietic progenitors comprise CD34+/CD43+/CD45+ cells.

97. The method of any one of claims 95-96, wherein the BMP pathway activator is BMP4.

98. The method of any one of claims 95-97, wherein the FGF is FGF2.

99. The method of any one of claims 95-98, wherein the VEGF is VEGF-165.

100. The method of claim 99, wherein the ROCK inhibitor is Y27632.

101. The method of any one of claims 95-100, wherein the differentiation media comprises stem cell factor (SCF).

102. The method of any one of claims 95-101, wherein the differentiation media comprises thrombopoietin (TPO).

103. The method of any one of claims 95-102, wherein the differentiation media comprises a low-density lipoprotein (LDL).

104. The method of any one of claims 95-103, wherein the differentiation media comprises a phosphoinositide 3-kinase (PI3K) inhibitor.

105. The method of claim 104, wherein the PI3K inhibitor is LY294002.

106. The method of any one of claims 95-105, wherein the differentiation media comprises a pyrimido-[4,5-b]-indole derivative.

107. The method of claim 106, wherein the pyrimido-[4,5-b]-indole derivative is UM729.

108. The method of any one of claims 95-107, wherein the differentiation media comprises an aryl hydrocarbon receptor (AhR) antagonist.

109. The method of claim 108, wherein the AhR antagonist is StemRegenin 1 (SR1).

110. The method of any one of claims 95-109, wherein the differentiation media comprises the BMP pathway activator, the FGF, the VEGF, and the ROCK inhibitor.

111. The method of any one of claims 95-110, wherein the differentiation media comprises the BMP pathway activator, the FGF, the VEGF, SCF, TPO, the LDL and the inhibitor of PI3K.

112. The method of any one of claims 95-111, wherein the differentiation media comprises the BMP pathway activator, the FGF, the VEGF, SCF, TPO, the LDL, the PI3K inhibitor, the pyrimido-[4,5-b]-indole derivative, and the AhR antagonist.

113. The method of any one of claims 95-112, comprising contacting the population of stem cells with the differentiation media for 1-5 days, wherein the differentiation media comprises the BMP pathway activator, the FGF, the VEGF, and optionally the ROCK inhibitor.

114. The method of any one of claims 95-113, comprising (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, and the PI3K inhibitor.

115. The method of any one of claims 95-114, comprising (i) contacting the population of stem cells for 1-5 days with the differentiation media comprising the BMP pathway activator, the FGF, the VEGF the ROCK inhibitor, to generate embryoid bodies or mesoderm cells, and (ii) contacting the embryoid bodies or mesoderm cells for 1-15 days with a differentiation media comprising the BMP pathway activator, the FGF, the VEGF, SCF, TPO, the LDL, the PI3K inhibitor, the pyrimido-[4,5-b]-indole derivative, and the AhR antagonist.

116. The method of any one of claims 95-115, wherein the differentiation media comprises 1-50 ng/mL BMP4, 1-50 ng/mL FGF2, 5-100 ng/mL VEGF, 0.1-20 uM ROCK inhibitor, 1-200 ng/mL SCF, 1-100 ng/mL TPO, 1-50 ug/mL LDL, 0.1-100 PI3K inhibitor, 0.1-10 uM pyrimido-[4,5-b]-indole derivative, 0.1-10 uM AhR antagonist, and any combination thereof.

117. The method of any one of claims 95-116, wherein the population of stem cells is a population of induced pluripotent stem cells (iPSCs).

118. The method of any one of claims 95-117, wherein the population of stem cells is a population of human embryonic stem cells (hESCs).

119. The method of any one of claims 95-118, wherein the yield ratio of hematopoietic progenitor cells from the population of stem cells is about 2:1 to about 10:1.

120. The method of any one of claims 95-119, wherein the yield ratio of the population of hematopoietic progenitor cells from a stem cell is about 5:1.

121. A kit to increase the yield ratio of NK cells from a population of stem cells, wherein the kit comprises instructions for differentiating the population of stem cells into NK cells and:
(a) a first media comprising a BMP pathway activator, an FGF, a VEGF, and optionally an inhibitor of ROCK;
(b) a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor;
(c) a second differentiation media comprising SCF, IL-7, IL-12, IL-15, FLT3L, a pyrimido-[4,5-b]-indole derivative, and an AhR inhibitor.

122. The kit of claim 121, wherein the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

123. The kit of claim 121 or 122, wherein each media of (a)-(c) is serum free.

124. The kit of any one of claims 121-123, wherein the media is xenogenic-free.

125. The kit of any one of claims 121-124, where the first media, the first differentiation media, and the second differentiation media each comprise the same base media.

126. The kit of any one of claims 121-125, where the first media, the first differentiation media, and the second differentiation media each comprise different base media.

127. The kit of any one of claims 121-126, where the first differentiation media and the second differentiation media each comprise the same base media, and the first media comprises a base media different from the first and second differentiation media.

128. The kit of any one of claims 121-127, wherein the first differentiation media and the second differentiation media each comprise a base media comprising Iscove's modified dulbecco's medium, bovine serum albumin, recombinant human insulin, human transferrin, and 2-mercaptoethanol.

129. The kit of any one of claims 121-128, further comprising a maturation media comprising (i) IL-12, IL-15 and IL-18, or (ii) IL-12, IL-2 and IL-18.

130. The kit of any one of claims 121-129, wherein the population of stem cells is a population of induced pluripotent stem cells (iPSCs) or a population of human embryonic stem cells (hESCs).

131. The kit of any one of claims 121-130, wherein the NK cells comprises about 60% to about 100% CD43+/CD45+/CD56+/LFA1+ cells.

132. The kit of any one of claims 121-131, wherein the population of stem cells is genetically engineered or edited.

133. The kit of any one of claims 121-132, wherein the NK cells are genetically engineered or edited.

134. The kit of any one of claims 121-133, wherein the yield ratio of NK cells from a population of stem cells is about 2:1 to about 100:1.

135. The kit of any one of claims 121-134, wherein the yield ratio of NK cells from a population of stem cells is about 35:1.

136. A method to increase the yield ratio of NK cells from a population of stem cells, comprising:
(a) obtaining a population of stem cells;
(b) contacting the population of stem cells with a first media comprising a BMP pathway activator, an FGF, a VEGF, and optionally an inhibitor of ROCK, for a period of time sufficient to generate embryoid bodies;
(c) contacting the embryoid bodies with a first differentiation media comprising a BMP pathway activator, a FGF, VEGF, SCF, TPO, an LDL, an inhibitor of PI3K, and optionally a pyrimido-[4,5-b]-indole derivative, and an AhR inhibitor, for a period of time sufficient to generate a population of hematopoietic progenitors;
(d) contacting the population of hematopoietic progenitors with a second differentiation media comprising SCF, IL-7, IL-12, IL-15, FLT3L, a pyrimido-[4,5-b]-indole derivative, and an AhR inhibitor, for a period of time sufficient to generate the NK cells.

137. The method of claim 136, wherein the BMP pathway activator is BMP4, the FGF is FGF2, the VEGF is VEGF-165, the inhibitor of ROCK is Y27632, the inhibitor of PI3K is LY294002, and the pyrimido-[4,5-b]-indole derivative is UM729.

138. The method of claim 136 or 137, wherein each media of steps (b)-(d) is serum free.

139. The method of any one of claims 136-138, wherein the method is xenogenic-free.

140. The method of any one of claims 136-139, where the first media, the first differentiation media, and the second differentiation media each comprise the same base media.

141. The method of any one of claims 136-139, where the first media, the first differentiation media, and the second differentiation media each comprise different base media.

142. The method of any one of claims 136-139, where the first differentiation media and the second differentiation media each comprise the same base media, and the first media comprises a base media different from the first and second differentiation media.

143. The method of any one of claims 136-139, wherein the first differentiation media and the second differentiation media each comprise a base media comprising Iscove's modified dulbecco's medium, bovine serum albumin, recombinant human insulin, human transferrin, and 2-mercaptoethanol.

144. The method of any one of claims 136-143, wherein the period of time of step (b) is 1-5 days, the period of time of step (c) is 3-15 days, and the period of time of step (d) is 11-25 days.

145. The method of any one of claims 136-144, wherein steps (a)-(d) occur within 35-45 days.

146. The method of any one of claims 136-145, comprising (e) expanding the NK cells with a maturation media comprising (i) IL-12, IL-15 and IL-18, or (ii) IL-12, IL-2 and IL-18.

147. The method of any one of claims 136-146, wherein the population of stem cells are induced pluripotent stem cells (iPSCs) or human embryonic stem cells (hESCs).

148. The method of any one of claims 136-147, wherein the population of hematopoietic progenitors comprises about 30% to about 50% CD34+/CD43+/CD45+ cells.

149. The method of any one of claims 136-148 wherein the NK cells comprise about 60% to about 100% CD43+/CD45+/CD56+/LFA1+ cells.

150. The method of any one of claims 136-149, comprising expanding the NK cells, wherein the NK cells expands about 10 to about 350 fold.

151. The method of any one of claims 136-150, wherein the population of stem cells are genetically engineered or edited.

152. The method of any one of claims 136-151, wherein the NK cells are genetically engineered or edited.

153. The method of any one of claims 136-152, wherein the yield ratio of NK cells from the population of stem cells is about 2:1 to about 100:1.

154. The method of any one of claims 136-153, wherein the yield ratio of NK cells from the population of stem cells is about 35:1.


SEQUENCE LISTING (15 sequences)

SEQ ID NO: 1 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 5 - protein, 107 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, synthetic construct [sequence omitted]

SEQ ID NO: 7 - protein, 93 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 8 - protein, 21 aa, synthetic construct
VVISVGSMGLIISLLCVYFWL

SEQ ID NO: 9 - protein, 19 aa, synthetic construct
VAVAGCVFLLISVLLLSGL

SEQ ID NO: 10 - protein, 25 aa, synthetic construct
PILLTISILSFFSVALLVILACVLW

SEQ ID NO: 11 - protein, 21 aa, synthetic construct
GWNPHLLLLLLLVIVFIPAFW

SEQ ID NO: 12 - protein, 21 aa, synthetic construct
MALPVTALLLPLALLLHAARP

SEQ ID NO: 13 - DNA, 1554 bp, synthetic construct
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 517 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1560 bp, synthetic construct
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa